(19) United States
(12) Patent Application Publication
(10) Pub. No.: US 2014/0357525 A1
(43) Pub. Date: Dec. 4, 2014

(54) MARKERS FOR ALZHEIMER'S DISEASE AND MILD COGNITIVE IMPAIRMENT AND METHODS OF USING THE SAME

(71) Applicant: Duke University, Durham, NC (US)

(72) Inventors: Rima F. Kaddurah-Daouk, Durham, NC (US); Pudugramam M. Doraiswamy, Durham, NC (US); Hongjie Zhu, Durham, NC (US); Steve Rozen, Durham, NC (US); Michael A. Kling, Durham, NC (US); John Q. Trajonowski, Durham, NC (US); Steven Arnold, Durham, NC (US)

(21) Appl. No.: 14/226,601

(22) Filed: Mar. 26, 2014

Related U.S. Application Data
(60) Provisional application No. 61/805,264, filed on Mar. 26, 2013.

(51) Int. Cl. G01N 33/68

(52) U.S. Cl. CPC G01N 33/6896; G01N 33/6806; G01N 2570/00; G01N 2800/2814; G01N 2800/2821
USPC 506/12; 436/89

(57) ABSTRACT

Disclosed herein are markers for diagnosing Alzheimer's Disease and/or mild cognitive impairment, for predicting risk of developing Alzheimer's Disease and/or mild cognitive impairment, and for monitoring the efficacy of treatment for Alzheimer's Disease and/or mild cognitive impairment. Also disclosed herein are methods of diagnosing Alzheimer's Disease and/or mild cognitive impairment in a subject in need thereof, for predicting risk of developing Alzheimer's Disease and/or mild cognitive impairment in a subject in need thereof, and for monitoring the efficacy of a treatment for Alzheimer's Disease and/or mild cognitive impairment in the subject.

### CROSS REFERENCE TO RELATED APPLICATION

This application claims priority to U.S. Prov. Pat. App. No. 61/805,264, filed Mar. 26, 2013, all of which is hereby incorporated by reference.

### STATEMENT OF GOVERNMENT INTEREST

This invention was made with government support under contract number R01 NS054008, R24GM078233, RC2 5RC2GM092729, P30 AG010124, and AG09215 awarded by the National Institutes of Health. The government has certain rights in the invention.

### TECHNICAL FIELD

The present invention relates to markers for diagnosing Alzheimer's Disease and/or mild cognitive impairment, to markers for predicting risk of developing Alzheimer's Disease and/or mild cognitive impairment, to markers for monitoring the efficacy of a treatment for Alzheimer's Disease and/or mild cognitive impairment, to methods of diagnosing Alzheimer's Disease and/or mild cognitive impairment, to methods of predicting risk of developing Alzheimer's Disease and/or mild cognitive impairment, and to methods for monitoring the efficacy of a treatment for Alzheimer's Disease and/or mild cognitive impairment.

### BACKGROUND

Alzheimer's Disease is an irreversible, progressive disease of the brain that destroys memory and thinking skills, including eventually the ability to carry out simple tasks. Alzheimer's Disease may be early-onset or late-onset. Early-onset Alzheimer's Disease is typically familial Alzheimer's Disease and occurs in individuals age 30 to 60. Late-onset Alzheimer's Disease is more common, usually developing after age 60, and has been linked to apolipoprotein E (APOE) gene. Many other factors may or may not contribute to the development and/or progression of Alzheimer's Disease, for example, diet, physical activity, social engagement, and associations between cognitive decline and vascular and metabolic conditions (e.g., heart disease, stroke, diabetes, obesity, etc.).

The disease process begins many years in advance of the appearance of symptoms, in which abnormal protein deposits form amyloid plaques and tau tangles throughout the brain. Neurons also begin to lose their ability to function and communicate with each other, and eventually die. The first symptoms include memory loss and some individuals display mild cognitive impairment (MCI). Individuals with MCI display more memory problems than normal for their age, but their symptoms are not as severe as the symptoms observed in individuals with Alzheimer's Disease. Individuals with MCI are more likely to go on and develop Alzheimer's Disease than those individuals without MCI. Once symptoms of Alzheimer's Disease appear (e.g., confusion, irritability, aggression, mood swings, trouble with language, and memory loss), the disease progresses from mild to moderate to severe, in which memory and cognitive abilities continue to decline.

Accordingly, a need exists for the identification and development of markers for risk prediction and/or detection of Alzheimer's Disease and/or mild cognitive impairment, especially early detection, to facilitate clinical treatment and management of disease progression.

### SUMMARY

The present invention is directed to a method of diagnosing cognitive impairment in a subject in need thereof. The method may comprise (a) obtaining a sample from the subject and (b) measuring a level of one or more metabolites in the sample. The measuring step may include an analytical tool to measure or detect the level or presence of the one or more metabolites in the sample. The analytical tool may be selected from the group consisting of a mass spectrometer, a nuclear magnetic resonance spectrometry, a gas chromatography instrument, an ion source, a mass analyzer, a detector capable of measuring mass-to-charge ratio of ions, a source of a magnetic field, a probe, an electrochemical detector, and a combination thereof.

The method may also comprise (c) comparing the level measured in step (b) with a level of the one or more metabolites in a control. A change in the level of the one or more metabolites as compared to the control may indicate that the subject is suffering from cognitive impairment. The one or metabolites may be in a pathway selected from the group consisting of a metabolic pathway, a tryptophan pathway, a tyrosine pathway, a purine pathway, a cysteine and methionine pathway, and any combination thereof.

The method may further comprise administering a therapeutically effective amount of an agent to the subject diagnosed with cognitive impairment.

The sample may be a cerebrospinal fluid sample. The cognitive impairment may be selected from the group consisting of Alzheimer's Disease and mild cognitive impairment.

The pathway may be the tryptophan pathway and the one or more metabolites may be selected from the group consisting of tryptophan (TRP), 5-hydroxyindoleacetic acid (5-HIAA), 5-hydroxytryptophan (5-HTP), kynurenine (KYN), indole-3-acetic acid (I-3-AA), and any combination thereof. The one or more metabolites may be 5-HIAA and an increase in the level of 5-HIAA as compared to the control may indicate that the subject is suffering from cognitive impairment. An increase in the level of I-3-AA, an increase in the level of KYN, or a decrease in the level of TRP as compared to the control may indicate that the subject is suffering from mild cognitive impairment. The one or more metabolites may be 5-HIAA and 5-HTP and an increase in a ratio of the levels of 5-HIAA:5-HTP as compared to the control may indicate that the subject is suffering from cognitive impairment. An increase in a ratio of the levels of KYN:TRP, an increase in a ratio of the levels of I-3-AA:TRP, or a decrease in a ratio of the levels of 5-HTP:TRP as compared to the control may indicate that the subject is suffering from mild cognitive impairment.

The method may further comprise (d) comparing the level measured in step (b) with a level of the one or more metabolites in a subject suffering from mild cognitive impairment and (e) determining the subject is suffering from Alzheimer's disease when the level measured in step (b) is lower than the level of the one or more metabolites in the subject suffering from mild cognitive impairment. The one or more metabolites may be 5-HTP.

The pathway may be the tyrosine pathway, the one or more metabolites may be vanillylmadelic acid (VMA), and an increase in the level of VMA as compared to the control may indicate that the subject is suffering from Alzheimer's Disease.

The pathway may be the purine pathway, the one or more metabolites may be xanthosine (XANTH), and an increase in the level of XANTH as compared to the control may indicate that the subject is suffering from Alzheimer's Disease.

The pathway may be the purine pathway, the one or more metabolites may be selected from the group consisting of hypoxanthine (HX) and uric acid (URIC), and an increase in the level of HX or URIC as compared to the control may indicate that the subject is suffering from mild cognitive impairment.

The pathway may be the purine pathway, the one more metabolites may be selected from the group consisting of uric acid (URIC), xanthine (XAN), xanthosine (XANTH), and hypoxanthine (HX), and an increase in a ratio of the levels of URIC:XAN, an increase in a ratio of the levels of XAN:XANTH, or a decrease in a ratio of the levels of XAN:HX as compared to the control may indicate that the subject is suffering from mild cognitive impairment.

The pathway may be the cysteine and methionine pathway, the one or more metabolites may be selected from the group consisting of methionine (MET) and glutathione (GSH), and an increase in the level of MET or a decrease in a ratio of the levels of GSH:MET as compared to the control may indicate that the subject is suffering from cognitive impairment.

The one or more metabolites may be selected from the group consisting of 15-65.533 and 8-93.65 and an increase in the level of 15-65.533 or an increase in the level of 8-93.65 as compared to the control may indicate that the subject is suffering from Alzheimer's Disease.

The method may further comprise (d) comparing the level measured in step (b) with a level of the one or more metabolites in a subject suffering from mild cognitive impairment and (e) determining the subject is suffering from Alzheimer's disease when the level measured in step (b) is lower than the level of the one or more metabolites in the subject suffering from mild cognitive impairment. The one or more metabolites may be selected from the group consisting of 12.94.5, 8.93.65, 8.89.433, 9.29.925, and 8.14.983.

The present invention is also directed to a method for monitoring an efficacy of a treatment of cognitive impairment in a subject. The method may comprise (a) obtaining a first sample from the subject before the treatment and a second sample from the subject during or after treatment. The method may also comprise (b) measuring a first level of a metabolite in the first sample and a second level of the metabolite in the second sample, wherein (i) the metabolite is selected from the group consisting of TRP, 5-HTP:TRP, XAN:HX and GSH:MET; or (ii) the metabolite is selected from the group consisting of 5-HIAA, I-3-AA, KYN, 5-HIAA:5-HTP, KYN:TRP, I-3-AA:TRP, VMA, XANTH, HX, URIC, URIC:XAN, XAN:XANTH, MET, 15-65.533, and 8-93.65. The method may also comprise (c) comparing the first level of the metabolite and the second level of the metabolite wherein (i) a second level of the metabolite of (b)(i) during or after treatment may be higher than the first level of the metabolite of (b)(i) before treatment and may be indicative of a therapeutic effect of the treatment in the subject; or (ii) a second level of the metabolite of (b)(ii) during or after treatment may be lower than the first level of the metabolite of (b)(ii) before treatment and may be indicative of a therapeutic effect of the treatment in the subject. The cognitive impairment may be selected from the group consisting of Alzheimer's Disease and mild cognitive impairment.

The present invention is also directed to a kit for diagnosing cognitive impairment in a subject, the kit comprising reagents for detecting one or more metabolites selected from the group consisting of 5-HIAA, 5-HTP, I-3-AA, KYN, TRP, VMA, XANTH, XAN, URIC, HX, MET, GSH, 15-65.533, 8-93.65, 12.94.5, 8.93.65, 8.89.433, 9.29.925, and 8.14.983. The cognitive impairment may be selected from the group consisting of Alzheimer's Disease and mild cognitive impairment.

### BRIEF DESCRIPTION OF THE DRAWINGS

FIG. 1 shows a schematic summarizing the changes in Alzheimer's Disease in the (A) methionine and cysteine pathway; (B) tryptophan pathway; (C) purine pathway; and (D) tyrosine pathway.

FIG. 2 shows a schematic summarizing the changes in mild cognitive impairment (MCI) in the (A) methionine and cysteine pathway; (B) tryptophan pathway; (C) purine pathway; and (D) tyrosine pathway.

FIG. 3 shows (A) a partial least square-discriminant analysis (PLS-DA) model for separation between Alzheimer's Disease (A) and normal cognition (C); PLS-DA model for separation between mild cognitive impairment (M) and normal cognition (C); (C) PLS-DA model cross-validation for Alzheimer's Disease (A) versus normal cognition (C); and (D) PLS-DA model cross-validation for mild cognitive impairment (M) versus normal cognition (C).

FIG. 4 shows a schematic illustrating a partial correlation network among clinical Alzheimer's Disease markers (e.g., amyloid-beta (Aβ-42), total tau (t-tau) and phosphorylated tau (p-tau)), mini-mental state exam (MMSE), and metabolites found in cerebrospinal fluid.

FIG. 5 shows box-plot distributions of metabolites 15-65.533 and 8-93.65 in relative concentrations for subjects with Alzheimer's Disease and control subjects (i.e., subjects without Alzheimer's Disease).

FIG. 6 shows a graph plotting 1-specificity against average sensitivity for stepwise logistic regression models across all cross-validation intervals for Alzheimer's Disease vs. control and normal modeling, considering all combinations of data types phosphorylated proteins (P), GC-TOF mass spectrometry metabolites (M), and liquid chromatography electrochemical array (LC-ECA) metabolites (E).

### DETAILED DESCRIPTION

The present invention relates to markers for diagnosing a cognitive impairment such as Alzheimer's Disease and/or mild cognitive impairment in a subject in need thereof. The markers can include factors. The present invention also relates to a method of identifying factors of Alzheimer's Disease and/or mild cognitive impairment in the subject. The method includes obtaining a sample from the subject and measuring or detecting a level of the factor in the sample either alone or in combination with one, two, three, or more factors.

The factor may be a metabolite from a metabolic or biochemical pathway for example, but not limited to, a tryptophan pathway, a tyrosine pathway, a purine pathway, and cysteine and methionine pathway. The level of the factor may be significantly changed (i.e., increased or decreased) in a subject suffering from Alzheimer's Disease and/or mild cognitive impairment. The level of the factor may be significantly changed (i.e., increased or decreased) in a subject at risk of developing Alzheimer's Disease and/or mild cognitive impairment. Accordingly, measurement of the factor level in the sample obtained from the subject may allow for the detection of Alzheimer's Disease and/or mild cognitive impairment in the subject both before and after the onset of clinical symptoms of Alzheimer's Disease and/or mild cognitive impairment.

The present invention also relates to a method for diagnosing Alzheimer's Disease and/or mild cognitive impairment in the subject, to a method for predicting risk of developing Alzheimer's Disease and/or mild cognitive impairment in the subject, and to a method for monitoring the efficacy of a treatment of Alzheimer's Disease and/or mild cognitive impairment in the subject. Such methods may utilize the method of identifying factors described above. For example, the method of diagnosing Alzheimer's Disease and/or mild cognitive impairment may compare a level of the factor measured in the sample obtained from the subject and a level of the factor measured in a control sample to determine if the subject is suffering from Alzheimer's Disease and/or mild cognitive impairment. The method of predicting risk of developing Alzheimer's Disease and/or mild cognitive impairment may compare a level of the factor measured in the sample obtained from the subject and a level of the factor measured in a control sample to determine if the subject is at risk of developing Alzheimer's Disease and/or mild cognitive impairment. Similar to the method of diagnosing Alzheimer's Disease and/or mild cognitive impairment, the method of monitoring can compare levels of the factor before and after treatment to evaluate the efficacy of the treatment in the subject.

The present invention also relates to a method for treatment of Alzheimer's Disease and/or mild cognitive impairment in the subject.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “control sample” or “control” as used herein means a sample or specimen taken from a subject, or an actual subject who does not have Alzheimer's Disease and/or mild cognitive impairment, or is not at risk of developing Alzheimer's Disease and/or mild cognitive impairment.

The term “effective dosage” or “therapeutically effective amount” as used herein means a dosage or amount of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual.

The term “metabolite” as used herein means a substance formed in or necessary for metabolism. Such substances may be, for example, but are not limited to, small molecules, cofactors of enzymes, and intermediates and products of metabolism.

The term “sample,” “test sample,” “specimen,” “biological sample,” “sample from a subject,” or “subject sample” as used herein interchangeably, means a sample or isolate of blood, tissue, urine, serum, plasma, salvia, amniotic fluid, cerebrospinal fluid, eye tissue, intraocular fluids, lens tissue, placental cells or tissue, endothelial cells, leukocytes, or monocytes, that can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

The term also means any biological material being tested for and/or suspected of containing an analyte of interest. The sample may be any tissue sample taken or derived from the subject. In some embodiments, the sample from the subject may comprise protein. In some embodiments, the sample from the subject may comprise nucleic acid. In still other embodiments, the sample from the subject may comprise one or more metabolites. Any cell type, tissue, or bodily fluid may be utilized to obtain a sample. Such cell types, tissues, and fluid may include sections of tissues such as biopsy and autopsy samples, frozen sections taken for histological purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include muscle tissue or fibres, lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary.

Methods well-known in the art for collecting, handling and processing muscle tissue or fibre, urine, blood, serum and plasma, cerebrospinal fluid, and other body fluids, are used in the practice of the present disclosure. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a cerebrospinal fluid or whole blood sample obtained from a subject. It may be necessary or desired that a test sample, particularly cerebrospinal fluid or whole blood, be treated prior to a method as described herein, e.g., with a pretreatment reagent. Even in cases where pretreatment is not necessary (e.g., most urine samples, a pre-processed archived sample, etc.), pretreatment of the sample is an option that can be performed for mere convenience (e.g., as part of a protocol on a commercial platform). The sample may be used directly as obtained from the subject or following pretreatment to modify a characteristic of the sample. Pretreatment may include extraction, concentration, inactivation of interfering components, and/or the addition of reagents.

The term “subject” or “patient” as used herein interchangeably, means any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc)) and a human. In some embodiments, the subject or patient may be a human or a non-human. The subject or patient may be undergoing other forms of treatment. In some embodiments, the subject or patient may be a human subject at risk for developing or already having Alzheimer's Disease and/or mild cognitive impairment.

“Treat”, “treating” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an agent of the present invention to a subject that is not at the time of administration afflicted with the disease. “Preventing” also refers to preventing the recurrence of a disease or of one or more symptoms associated with such disease. “Treatment” and “therapeutically” refer to the act of treating, as “treating” is defined above.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. METHOD OF IDENTIFYING FACTORS OF COGNITIVE IMPAIRMENT

Provided herein is a method of identifying factors of cognitive impairment in a subject in need thereof. Cognitive impairment may include, but is not limited to, Alzheimer's Disease (AD) and/or mild cognitive impairment (MCI).

The method includes obtaining a sample from the subject and measuring or detecting a level of the factor in the sample either alone or in combination with one, two, three, or more factors. A change in the level of the factor in the sample obtained from the subject relative to a control sample identifies the factor of cognitive impairment, thereby indicating that the subject is suffering from cognitive impairment. The change in the level of the factor may be an increase in the level of or a presence of the factor in the sample obtained from the subject. Alternatively, the change in the level of the factor may be a decrease in the level of or an absence of the factor in the sample obtained from the subject.

The level of the factor may be increased by at least about 0.5-fold, 1.0-fold, 1.5-fold, 2.0-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold or greater in the sample obtained from the subject relative to the control sample. In other embodiments, the level of the factor may decreased by at least about 0.5-fold, 1.0-fold, 1.5-fold, 2.0-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold or greater in the sample obtained from the subject relative to the control sample.

In addition to the level of the factor, a mini-mental state exam (MMSE), a cognitive score, a level of amyloid-beta, a level of total tau (t-tau), and/or a level of phosphorylated tau (p-tau) may help to predict the risk of the subject developing cognitive impairment. A method for predicting risk of developing cognitive impairment is described in more detail below.

The method may further comprise administering a therapeutically effective amount of an agent to the subject suffering from, diagnosed with, or predicted to be at risk of developing cognitive impairment as described below in more detail.

a. Factor

The method may identify one, two, three, or more factors of cognitive impairment alone or in combination in the sample obtained from the subject in need thereof. The method may measure or detect the change in the level of the factor in the sample alone or in combination with one, two, three, or more factors.

The factor may be a metabolite. The metabolite may be a lipid. The metabolite may be a co-factor for an enzyme or protein in a metabolic pathway. The metabolite may be a co-factor for an enzyme or protein. The metabolite may be a substrate of an enzyme or protein. The metabolite may be a beginning product, an intermediate, or an end product in a metabolic pathway. The metabolite may be a metabolite described in the Kyoto Encyclopedia of Genes and Genomes (KEGG) database.

The factor may be a metabolite in a metabolic pathway, for example, but not limited to, a tryptophan pathway, a tyrosine pathway, a purine pathway, a cysteine and methionine pathway, a phenylalanine pathway, or another biochemical pathway. The metabolic pathway may be a metabolic pathway described in the Kyoto Encyclopedia of Genes and Genomes (KEGG) database.

The factor may be a ratio of two metabolites in the same metabolic pathway. The factor may be a ratio of two metabolites in different branches in a metabolic pathway. The factor may be a ratio of two metabolites in the same branch in a metabolic pathway. The factor may be a ratio of two metabolites, in which the first metabolite is not in the same metabolic pathway as the second metabolite.

The factor may be a ratio of two or more metabolites in the same metabolic pathway. The factor may be a ratio of two or more metabolites, in which the two or more metabolites are not necessarily in the same branches of a metabolic pathway. The factor may be a ratio of two or more metabolites, in which the two or more metabolites are in the same branch of a metabolic pathway. The factor may be a ratio of two or more metabolites, in which the two or more metabolites are not necessarily in the same metabolic pathways.

(1) Tryptophan Pathway

The factor may be the metabolite in the tryptophan pathway. The metabolite in the tryptophan pathway may be any metabolite in the tryptophan pathway, including, for example, but not limited to, any metabolites in the tryptophan pathway described in the Kyoto Encyclopedia of Genes and Genomes (KEGG) database. The metabolite in the tryptophan pathway may be a beginning product, an intermediate, or an end product in the tryptophan pathway. The metabolite in the tryptophan pathway may be tryptophan (TRP), 5-hydroxyindoleacetic acid (5-HIAA), 5-hydroxytryptopah (5-HTP), kynurenine (KYN), or indole-3-acetic acid (I-3-AA). 5-HIAA is a major metabolite of serotonin (5-HT).

The factor may a ratio of two metabolites in the tryptophan pathway. The factor may be a ratio of two metabolites in different branches or portions of the tryptophan pathway. The factor may be a ratio of two metabolites in the same branch or portion of the tryptophan pathway. The factor may be a ratio of two metabolites, in which the first metabolite is in the tryptophan pathway and the second metabolite is not in the tryptophan pathway.

The factor may be a ratio of two or more metabolites in the tryptophan pathway. The factor may be a ratio of two or more metabolites, in which the two or more metabolites are not necessarily in the same branches or portions of the tryptophan pathway. The factor may be a ratio of two or more metabolites, in which at least one of the metabolites is not in the tryptophan pathway and at least one of the metabolites is in the tryptophan pathway.

The level of 5-HIAA may be increased in the subject suffering from Alzheimer's Disease as compared to a level of 5-HIAA in a subject not having Alzheimer's Disease (i.e., has normal cognition). The ratio of 5-HIAA:5-HTP may be increased in the subject suffering from Alzheimer's Disease as compared to a ratio of 5-HIAA:5-HTP in the subject not having Alzheimer's Disease.

The level of 5-HIAA may be increased in the subject suffering from mild cognitive impairment (MCI) as compared to a level of 5-HIAA in a subject not suffering from mild cognitive impairment. MCI is often found in subjects that progress to or develop Alzheimer's Disease. The level of I-3-AA may be increased in the subject suffering from MCI as compared to a level of I-3-AA in the subject not suffering from mild cognitive impairment. The level of KYN may be increased in the subject suffering from MCI as compared to a level of KYN in the subject not suffering from MCI. The level of TRP may be decreased in the subject suffering from MCI as compared to a level of TRP in the subject not suffering from MCI

The ratio of 5-HIAA:5-HTP may be increased in the subject suffering from MCI as compared to a ratio of 5-HIAA:5-HTP in the subject not suffering from MCI. The ratio of KYN:TRP may be increased in the subject suffering from MCI as compared to a ratio of KYN:TRP in the subject not suffering from MCI. The ratio of I-3-AA:TRP may be increased in the subject suffering from MCI as compared to a ratio of I-3-AA:TRP in the subject not suffering from MCI. The ratio of 5-HTP:TRP may be decreased in the subject suffering from MCI as compared to a ratio of 5-HTTP:TRP in the subject not suffering from MCI.

The level of 5-HTP may be lower (i.e., decreased) in the subject suffering from MCI as compared to the level of 5-HTP in the subject suffering from Alzheimer's Disease.

(2) Tyrosine Pathway

The factor may be the metabolite in the tyrosine pathway. The metabolite in the tyrosine pathway may be any metabolite in the tyrosine pathway, including, for example, but not limited to, any metabolites in the tyrosine pathway described in the Kyoto Encyclopedia of Genes and Genomes (KEGG) database. The metabolite in the tyrosine pathway may be a beginning product, an intermediate, or an end product in the tyrosine pathway. The metabolite in the tyrosine pathway may be 4-hydroxyphenylacetic acid (4-HPAC), homovanillic acid ((HVA), methoxyhydroxyphenlyglycol (MHPG), tyrosine (TYR), or vanillylmandelic acid (VMA). VMA is an end product of catecholamine metabolism.

The factor may a ratio of two metabolites in the tyrosine pathway. The factor may be a ratio of two metabolites in different branches or portions of the tyrosine pathway. The factor may be a ratio of two metabolites in the same branch or portion of the tyrosine pathway. The factor may be a ratio of two metabolites, in which the first metabolite is in the tyrosine pathway and the second metabolite is not in the tyrosine pathway.

The factor may be a ratio of two or more metabolites in the tyrosine pathway. The factor may be a ratio of two or more metabolites, in which the two or more metabolites are not necessarily in the same branches or portions of the tyrosine pathway. The factor may be a ratio of two or more metabolites, in which at least one of the metabolites is not in the tyrosine pathway and at least one of the metabolites is in the tyrosine pathway.

The level of VMA may be increased in the subject suffering from Alzheimer's Disease as compared to a level of VMA in the subject not suffering from Alzheimer's Disease.

(3) Purine Pathway

The factor may be the metabolite in the purine pathway. The metabolite in the purine pathway may be any metabolite in the purine pathway, including, for example, but not limited to, any metabolites in the purine pathway described in the Kyoto Encyclopedia of Genes and Genomes (KEGG) database. The metabolite in the purine pathway may be a beginning product, an intermediate, or an end product in the purine pathway. The metabolite in the purine pathway may be guanosine (GR), hypoxanthine (HX), uric acid (URIC), xanthine (XAN), xanthosine (XANTH), or paraxanthine (PXAN).

The factor may a ratio of two metabolites in the purine pathway. The factor may be a ratio of two metabolites in different branches or portions of the purine pathway. The factor may be a ratio of two metabolites in the same branch or portion of the purine pathway. The factor may be a ratio of two metabolites, in which the first metabolite is in the purine pathway and the second metabolite is not in the purine pathway.

The factor may be a ratio of two or more metabolites in the purine pathway. The factor may be a ratio of two or more metabolites, in which the two or more metabolites are not necessarily in the same branches or portions of the purine pathway. The factor may be a ratio of two or more metabolites, in which at least one of the metabolites is not in the purine pathway and at least one of the metabolites is in the purine pathway.

The level of XANTH may be increased in the subject suffering from Alzheimer's Disease as compared to a level of XANTH in the subject not suffering from Alzheimer's Disease. The level of HX may be increased in the subject suffering from MCI as compared to a level of HX in the subject not suffering from MCI. The level of URIC may be increased in the subject suffering from MCI as compared to a level of URIC in the subject not suffering from MCI.

The ratio of URIC:XAN may be increased in the subject suffering from MCI as compared to a ratio of URIC:XAN in the subject not suffering from MCI. The ratio of XAN:XANTH may be increased in the subject suffering from MCI as compared to a ratio of XAN:XANTH in the subject not suffering from MCI. The ratio of XAN:HX may be decreased in the subject suffering from MCI as compared to a ratio of XAN:HX in the subject not suffering from MCI.

(4) Cysteine and Methionine Pathway

The factor may be the metabolite in the cysteine and methionine pathway (also known as one carbon metabolic pathway or one carbon metabolism). The metabolite in the cysteine and methionine pathway may be any metabolite in the cysteine and methionine pathway, including, for example, but not limited to, any metabolites in the cysteine and methionine pathway described in the Kyoto Encyclopedia of Genes and Genomes (KEGG) database. The metabolite in the cysteine and methionine pathway may be a beginning product, an intermediate, or an end product in the cysteine and methionine pathway. The metabolite in the cysteine and methionine pathway may be a metabolite in the folate cycle (e.g., tetrahydro folic acid (THF) and folate) or the methionine cycle (e.g., methionine, S-adenosyl methionine, homocysteine, and S-adenosyl methionine) of the cysteine and methionine pathway. The metabolite in the cysteine and methionine pathway may be methionine (MET) or glutathione (GSH).

The factor may a ratio of two metabolites in the cysteine and methionine pathway. The factor may be a ratio of two metabolites in the methionine cycle. The factor may be a ratio of two metabolites in the folate cycle. The factor may be a ratio of two metabolites, in which one metabolite is in the methionine cycle and the other metabolite is in the folate cycle. The factor may be a ratio of two metabolites in different branches or portions of the cysteine and methionine pathway. The factor may be a ratio of two metabolites in the same branch or portion of the cysteine and methionine pathway. The factor may be a ratio of two metabolites, in which the first metabolite is in the cysteine and methionine pathway and the second metabolite is not in the cysteine and methionine pathway. The factor may be a ratio of two, in which one of the metabolite is in the cysteine and methionine pathway and the other metabolite is a lipid, a phospholipid, or a phosphotidylcholine. The factor may be a ratio of two, in which one of the metabolite is in the folate or methionine cycle and the other metabolite is a lipid, a phospholipid, or a phosphotidylcholine.

The factor may be a ratio of two or more metabolites in the cysteine and methionine pathway. The factor may be a ratio of two or more metabolites in the methionine cycle. The factor may be a ratio of two or more metabolites in the folate cycle. The factor may be a ratio of two or more metabolites, in which at least one metabolite is in the methionine cycle and at least one metabolite is in the folate cycle. The factor may be a ratio of two or more metabolites, in which the two or more metabolites are not necessarily in the same branches or portions of the cysteine and methionine pathway. The factor may be a ratio of two or more metabolites, in which at least one of the metabolites is not in the cysteine and methionine pathway and at least one of the metabolites is in the cysteine and methionine pathway. The factor may be a ratio of two or metabolites, in which at least one of the metabolites is in the cysteine and methionine pathway and at least one of the metabolites is a lipid, a phospholipid, or a phosphotidylcholine. The factor may be a ratio of two or metabolites, in which at least one of the metabolites is in the folate or methionine cycle and at least one of the metabolites is a lipid, a phospholipid, or a phosphotidylcholine.

The cysteine and methionine pathway (i.e., one carbon metabolism) may contribute to methylation steps in other metabolic pathways, for example, but not limited to, through the folate cycle of the cysteine and methionine pathway, the methionine cycle of the cysteine and methionine pathway, the co-factor (S-adenosyl methionine (SAM)), and a combination thereof. Accordingly, alterations or perturbations in the cysteine and methionine pathway may lead to alterations or perturbations in other metabolic pathways, for example, but not limited, pathways leading to the synthesis of neurotransmitters (e.g., catecholamines, norepinephrine, etc.), pathways leading to the synthesis of lipids or phospholipids, pathways leading to the synthesis of phosphotidylcholines, pathways in which a lipid or phospholipid is a co-factor or substrate of an enzyme or protein in the pathways or a beginning product, intermediate, or end product in the pathways. As such, alterations or perturbations in the cysteine and methionine pathway may alter the levels of other metabolites, for example, but not limited to, neurotransmitters (e.g., catecholamines), phosphatidylcholines, lipids, phospholipids (e.g., ceramides and sphingomyelins), or a combination thereof. In other embodiments, the cysteine and methionine pathway (i.e., one carbon metabolism) may contribute to methylation steps in the pathways that synthesize one or more of the lipids or phospholipids described in Han et al., “Metabolomics in Early Alzheimer's Disease: Identification of Altered Plasma Sphingolipidome using Shotgun Lipidomics,” (July 2011), PLos ONE, volume 6, issue 7, e21643, the entire contents of which are hereby incorporated by reference.

The level of MET may be increased in the subject suffering from Alzheimer's Disease as compared to a level of MET in the subject not suffering from Alzheimer's Disease. The ratio of GSH:MET may be decreased in the subject suffering from Alzheimer's Disease as compared to a ratio of GSH:MET in the subject not suffering from Alzheimer's Disease.

The level of MET may be increased in the subject suffering from MCI as compared to a level of MET in the subject not suffering from MCI. The ratio of GSH:MET may be decreased in the subject suffering from MCI as compared to a ratio of GSH:MET in the subject not suffering from MCI.

(5) Other Factors

The factor may be a metabolite identifiable by liquid chromatography electrochemical array (LC-ECA), for example, 15-65.533, 12-94.5, 8-93.65, 8-89.433, 14-64.275, 9-20.858, 9-29.925, 8-14.983, 5-40.292, 13-18.475, 8-63.675, 15-68.542, 8-93.65, 12-94.5, 5-40.292, 4-22.117, 8-3.675, 15-77.017, 8-89.433, and 15-90.6.

The metabolites 15-65.533 and 8-93.65 may discriminate between Alzheimer's Disease and normal cognition. Specifically, the levels of 15-65.533 and 8-93.65 may be higher in the subject suffering from Alzheimer's Disease as compared to the levels of 15-65.533 and 8-93.65 in the subject not suffering from Alzheimer's Disease.

The metabolites 12.94.5, 8.93.65, 8.89.433, 9.29.925, and 8.14.983 may discriminate between Alzheimer's Disease and mild cognitive impairment. Specifically, the levels of 12.94.5, 8.93.65, 8.89.433, 9.29.925, and 8.14.983 may be higher or elevated in the subject suffering from Alzheimer's Disease as compared to the levels of 12.94.5, 8.93.65, 8.89.433, 9.29.925, and 8.14.983 in the subject suffering from mild cognitive impairment.

The factor may be another metabolite, for example, but not limited to, serotonin, phenylalanine, proline, lysine, lysine, phosphatidylcholine (PC), taurine, acyl carnitine (AC), PC diacyl (aa) C36:6, PC aa C38:0, PC aa C38:6, PC aa C40:1, PC aa C40:2, PC aa C40:6, PC acyl-alkyl (ae) C40:6, lysophophatidylcholine (lyso PC a C18:2), and acylcarnitines (ACs).

b. Measurement or Detection of the Level of the Factor

As discussed above, the method may include measuring or detecting the level of the factor in the sample alone or in combination with one, two, three, or more factors. The level of the factor may be measured or detected by any means known in the art, for example, but not limited to analytical tools for metabolomics science. Analytical tools for metabolomics science may include, but are not limited to, mass spectrometry (MS), nuclear magnetic resonance (NMR), liquid chromatography electrochemical array (LC-ECA), and Fourier transform infrared spectrometry (FT-IR).

Mass spectrometry, as an analytical tool for measuring or detecting the level of the factor in the sample, may include an ion source, a mass analyzer, and/or a detector that is capable of measuring the mass-to-charge ratio of ions in compounds of the sample. Mass spectrometry may include different platforms, for example, but not limited to, gas chromatography mass spectrometry (GC-MS),2-dimensional gas chromatography mass spectrometry (GC×GC-MS), gas chromatography time of flight mass spectrometry (GC-TOF), high performance liquid chromatography mass spectrometry (HPCL-MS), ultra performance liquid chromatography mass spectrometry (UPLC-MS), and capillary electrophoresis mass spectrometry (CE-MS).

Nuclear magnetic resonance, as an analytical tool for measuring or detecting the level of factor in the sample, may include a source of a magnetic field (i.e., magnet) and/or a probe. Nuclear magnetic resonance may include different platforms, for example, but not limited to, ¹H-NMR, ¹³C-NMR, 31P-NMR, and liquid chromatography nuclear magnetic resonance (LC-NMR).

Liquid chromatography electrochemical array (LC-ECA), as an analytical tool for measuring or detecting the level of the factor in the sample, may include an electrochemical detector, an amperometric sensor, and/or a coulometric sensor.

Accordingly, the measuring step in the method may include the analytical tool to measure or detect the level, presence, or absence of the one or more metabolites in the sample. The analytical tool may be selected from the group consisting of a mass spectrometer, a nuclear magnetic resonance spectrometer, a gas chromatography instrument, an ion source, a mass analyzer, a detector capable of measuring mass-to-charge ratio of ions, a source of a magnetic field, a probe, an electrochemical detector, and a combination thereof.

### 3. METHOD OF DIAGNOSING COGNITIVE IMPAIRMENT

Also provided herein is a method of diagnosing cognitive impairment in a subject in need thereof. As discussed above, cognitive impairment may include Alzheimer's Disease and/or mild cognitive impairment. The method of diagnosing may apply the method of identifying factors of cognitive impairment described above to determine if the subject is suffering from cognitive impairment. The method of diagnosing may include obtaining a sample from the subject and measuring or detecting the level of one or more factors in the sample. The method of diagnosing may also include comparing the measured level of the one or more factors to a level of the factor in a control to determine if the subject is suffering from cognitive impairment.

### 4. METHOD OF PREDICTING RISK OF DEVELOPING COGNITIVE IMPAIRMENT

Also provided herein is a method of predicting risk of developing cognitive impairment in a subject in need thereof. As discussed above, cognitive impairment may include Alzheimer's Disease and/or mild cognitive impairment. The method of predicting risk may apply the method of identifying factors of cognitive impairment described above to determine if the subject is at risk of developing cognitive impairment. The method of predicting risk may include obtaining a sample from the subject and measuring or detecting the level of one or more factors in the sample. The method of predicting risk may also include comparing the measured level of the one or more factors to a level of the one or more factors in a control to determine if the subject is at risk of developing cognitive impairment.

The method of predicting risk may further include determining a mini-mental state exam (MMSE), a cognitive score, a level of amyloid-beta, a level of total tau (t-tau), a level of phosphorylated tau (p-tau), and a combination thereof in the subject. An altered (i.e., increased or decreased) MMSE, cognitive score, level of amyloid beta, level of t-tau, level of p-tau, or a combination thereof may further predict that the subject is at risk of developing cognitive impairment.

In some embodiments, a subject determined to be at risk of developing cognitive impairment by the method described herein may be selected for clinical research studies.

### 5. METHOD OF MONITORING EFFICACY OF TREATMENT OF COGNITIVE IMPAIRMENT

Also provided herein is a method of monitoring efficacy of treatment of cognitive impairment in a subject undergoing treatment of cognitive impairment in any form. As discussed above, cognitive impairment may include Alzheimer's Disease and/or mild cognitive impairment. The method of monitoring may apply the method of identifying factors of cognitive impairment described above to determine if the treatment of cognitive impairment has a therapeutic effect in the subject. The method of monitoring may include obtaining a first sample from the subject before treatment has begun and obtaining a second sample from the subject after treatment has begun. The levels of one or more factors may be measured or detected in the first and second samples to determine a first level and a second level of the one or more factors, respectively. The first and second levels of the one or more factors may be compared to determine if the second level is different or changed (e.g., higher or lower) from the first level, in which the difference indicates whether the cognitive impairment treatment has had a therapeutic effect in the subject.

### 6. METHOD OF TREATING AND/OR PREVENTING COGNITIVE IMPAIRMENT

Provided herein is a method for treating and/or preventing cognitive impairment in a subject in need thereof. As discussed above, cognitive impairment may include Alzheimer's Disease and/or mild cognitive impairment. The method includes administering a composition comprising a therapeutically effective amount of an agent to the subject diagnosed with cognitive impairment by the method described herein.

In the subject suffering from cognitive impairment, the agent can alter the level or activity of one or more of the factors discussed above in the subject such that the level or activity of the one or more factors in a sample obtained from subject after treatment has begun is substantially the same as a level or activity of the one or more factors in a control sample. The agent may reduce or alleviate symptoms of cognitive impairment in the subject administered the agent. The agent may delay the development of symptoms of cognitive impairment in the subject administered the agent. The agent may prevent symptoms of cognitive impairment in the subject administered the agent. The agent may delay or reduce the appearance of symptoms of cognitive impairment in the subject identified as being at risk of developing cognitive impairment by the methods described herein or another method. Symptoms may include, but are not limited to, memory loss, difficulties in completing routine or familiar tasks, challenges in planning or solving problems, confusion with time or place, trouble understanding visual images and spatial relationship, misplacement of items, changes in mood or personality (e.g., depression, mood swings, and irritability), and any combination thereof. The type of agent used in the method of treatment may depend on whether the subject is identified as having Alzheimer's Disease or mild cognitive impairment, for example.

The agent may prevent cognitive impairment in the subject or a subject identified as being at risk of developing cognitive impairment by the methods described herein or another method.

The agent may be, but is not limited to, a cholinesterase inhibitor (e.g., donepezil, rivastigmine, and galantamine), a N-methyl-D-aspartate (NMDA) antagonist (e.g., memantine), an over counter supplement or food product (e.g., fish oil, ginkgo, Axona, and vitamins), and any combination thereof

### 7. KIT

Also provided herein is a kit for use with the methods disclosed herein. The kit may include one or more reagents for detecting the factors either alone or in any combination thereof. The reagents for detecting the factors may be any of those reagents known in the art for detecting a metabolite, for example, but not limited to, reagents for mass spectrometry, reagents for nuclear magnetic resonance, reagents for liquid chromatography electrochemical array (LC-ECA), reagents for immunoassays (e.g., ELISA, western blotting, immunoprecipitation (IP)), and any combination thereof.

The kit may also include other material(s), which may be desirable from a user standpoint, such as a buffer(s), a diluent(s), a standard(s), and/or any other material useful in sample processing, washing, or conducting any other step of the methods described herein. The kit may further include one or more containers for holding or containing the reagents or other materials.

The kit may also include controls and/or instructions for using the kit (i.e., carrying out the methods disclosed herein). Instructions included in the kit may be affixed to packaging material or may be included as a package insert. While instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet set which provides instructions.

The present invention has multiple aspects, illustrated by the following non-limiting examples.

### 8. EXAMPLES

### Example 1

### Materials and Method for Examples 2-4

Study Design and Participants.

This case-control study examined participants enrolled in a prospective longitudinal study. The participants were recruited at the Penn Memory Center, University of Pennsylvania (Philadelphia, Pa., USA) and the Maria de los Santos Health Center (Philadelphia, Pa., USA), following written informed consent. Cases were classified as Alzheimer's Disease (AD) or mild cognitive impairment (MCI) based on standard diagnostic criteria. From this cohort, a subset of 114 participants (40 AD, 36 MCI and 38 CN) was identified who had banked cerebrospinal fluid (CSF) samples and other traditional biomarker data. Cases from each diagnostic category were matched as closely as possible for age and gender. Neuropsychological testing was conducted including the Clinical Dementia Rating, Dementia Rating Scale-Second Edition, Mini-Mental State Exam (MMSE) and/or tests of frontal executive function, memory, language, praxis, visuospatial construction, motor performance, mood and function. CSF sample collection and standardized Lumixex assay for amyloid-β (Ab42), total tau (t-tau) and phosphorylated tau (p-tau) at the threonine 181 were done by standard methods. There were no significant differences between AD, MCI and normal control (CN) groups with regard to age and gender; however, as expected, baseline cognitive status and apolipoprotein E (ApoE) ε4 genotype prevalence were significantly different (Table 1).

TABLE 1
Participant Demographics and Clinical Characteristics

| | AD (N = | MCI (N = | CN (N = | P- | |
|---|---|---|---|---|---|
| Characteristics | 40) | 36) | 38) | value | Test |
| Age range | 51.3-90.2 | 50.3-86.7 | 51.3-87.3 | | |
| Mean age | 69 | 69.9 | 69.5 | 0.93 | K |
| Male, no. (%) | 10 (25.0) | 17 (47.2) | 13 (34.2) | 0.13 | F |
| Median years of education ± MAD | 15.5 ± 3.0 | 14.5 ± 2.5 | 18.0 ± 2.0 | 0.003 | K |
| Median MMSE ± MAD | 23 ± 3.0 | 27 ± 2.0 | 30 ± 0 | <0.001 | K |
| Mean age onset ± s.d. | 65.3 ± 8.9 | 66.8 ± 9.6 | NA | 0.48 | T |
| with ApoE ε4, no. (%) | 23 (62.2) | 13 (37.1) | 12 (31.6) | 0.018 | F |
| Taking cholinesterase inhibitors, no. | 15 (37.5) | 8 (22.2) | 0 | <0.001 | F |
| (%) | | | | | |
| Taking memantine, no. (%) | 6 (15%) | 0 | 0 | 0.003 | F |

AD, Alzheimer's Disease;
ApoE, apolipoprotein E;
CN, normal cognition;
F, Fisher' exact test, two-sided;
K, Kruskal-Wallis test;
MAD, median absolute deviation;
MCI, mild cognitive impairment;
MMSE, Mini-Mental State Exam;
T, two-sided t-test between AD and MCI.

Metabolomic Profiling.

Samples were analyzed using a liquid chromatography electrochemical array platform. Levels of 71 metabolites, including 24 known compounds, were measured (see Table 2 for known compounds and their abbreviations).

TABLE 2
List of Known Compounds Quantified by the LC-ECA Pattern

| Metabolite by Pathways | Abbreviation |
|---|---|
| Tryptophan | |
| Tryptophan | TRP |
| 5-Hydroxyindoleacetic acid | 5-HIAA |
| 5-Hydroxytryptophan | 5-HTP |
| Kynurenine | KYN |
| Indole-3-acetic acid | I-3-AA |
| Tyrosine | |
| 4-Hydroxyphenylacetic acid | 4-HPAC |
| Homovanillic acid | HVA |
| Methoxyhydroxyphenlyglycol | MHPG |
| Tyrosine | TYR |
| Vanillylmandelic acid | VMA |
| Phenylalanine | |
| 4-Hydroxybenzoic acid | 4-HBAC |
| 4-Hydroxyphenyllactic acid | 4-HPLA |
| 2-Hydroxyphenylacetic acid | 2-HPAC |
| Purine | |
| Guanosine | GR |
| Hypoxanthine | HX |
| Uric acid | URIC |
| Xanthine | XAN |
| Xanthosine | XANTH |
| Paraxanthine | PXAN |
| Cysteine and Methionine | |
| Glutathione (reduced) | GSH |
| Methionine | MET |
| Other | |
| Ascorbic acid | ASA |
| Delta-tocopherol | DTOCO |
| Indole-3-propionic acid | I-3-PA |

LC-ECA, liquid chromatograpy electrochemical array.

Data Analysis.

Data analysis included univariate and multivariate statistical techniques. The Fisher's exact test was used to examine the association of the following clinical covariates with disease status: gender, with APOE ε4, cholinesterase inhibitors and memantine. Kruskal-Wallis tests were used to test between-diagnostic-group differences in age, years of education and MMSE scores. Two-sample t-test was used to compare age of onset between diagnostic groups. The raw metabolomics data were first viewed by quantile-quantile normal and χ2 plots, and by variable-pair scatterplots, to assess normality and nonlinear relationships. As most analytes were not approximately normally distributed, nonparametric Kruskal-Wallis tests were used for pairwise comparison between AD or MCI and CN. Significant metabolites were mapped to several key biochemical pathways. Differences among diagnostic groups in product/substrate ratios within the pathways were examined because the ratios of compounds may indicate the relative effectiveness of enzymes involved in the pathways. Correlations between metabolites and protein markers were obtained by calculating their Pearson's correlation coefficients. The significance of correlation was tested using Student's t-distribution. For all above systematic univariate tests, multiple comparison was corrected by estimating the positive false discovery rate using Storey's q-value. The partial correlation network was built among metabolites, protein markers and MMSE using the sparse partial correlation estimation approach. An edge between two network variables implies conditional dependency between corresponding variable pairs conditional on the rest of the variables. The false discovery rate was controlled at 0.05.

Metabolomic profiles were used to construct partial least square-discriminant analysis (PLS-DA) models for categorical separation of AD or MCI and CN. The variable importance in projection parameter was used to identify metabolites that make the most contribution in discriminating diagnostic groups in the PLS-DA models, and threefold cross-validation of the PLS-DA models was performed to evaluate model predictive performance. Participant data from different groups were randomly divided into training (about ⅔ of all participants in a given group) and test (remaining participants in a given group) sets. Following construction of PLS-DA models using training sets, the models were used to predict class membership of the test-set participants. This procedure was repeated three times with different participants in the training and test sets and a new PLS-DA model constructed each time.

### Example 2

### Metabolic Differences Between AD, MCI, and CN Groups

Metabolites and Pathways Altered in AD.

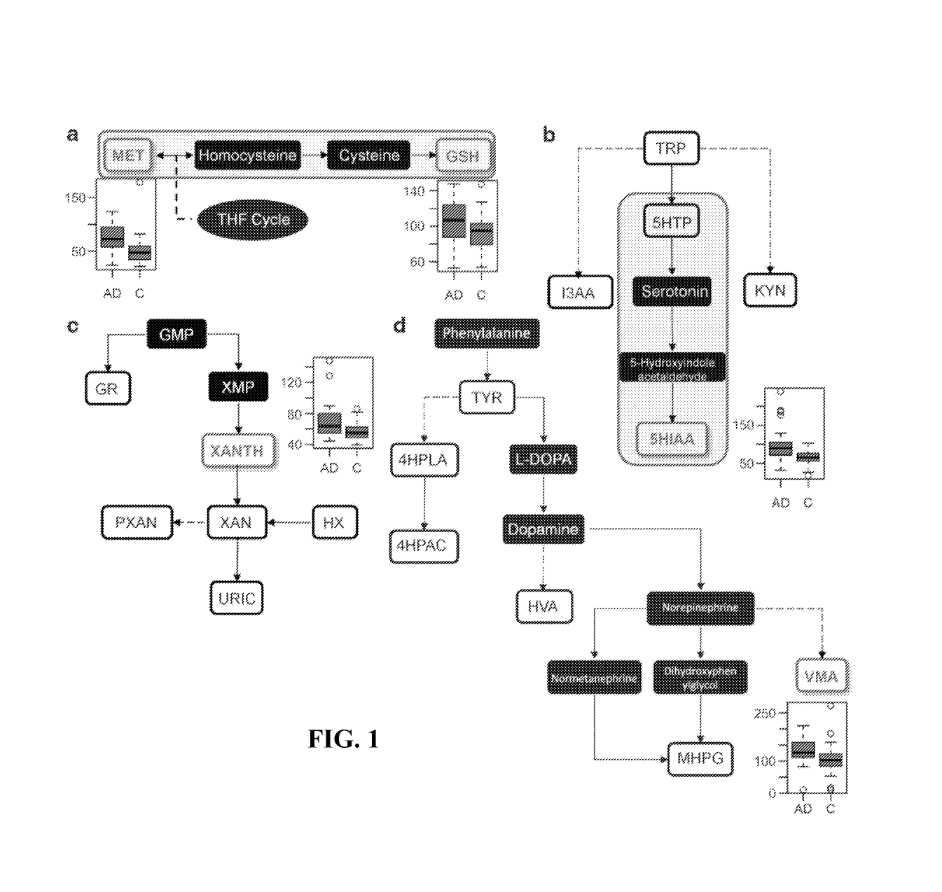

Several metabolites were significantly different in AD patients versus controls (Table 3 and FIG. 1). In FIG. 1, the metabolites in black boxes or circles (with white lettering) were not measured. Methionine (MET), involved in one-carbon metabolism and methylation processes; 5-Hydroxyindoleacetic acid (5-HIAA), a major metabolite of serotonin (5-HT); Vanillylmandelic acid (VMA), an end product of catecholamine metabolism; and Xanthosine (XANTH), a purine pathway metabolite, were significantly increased in AD. The 5-HIAA/5-Hydroxytryptophan (5-HTP) ratio was significantly increased in AD, whereas the GSH (glutathione)/MET ratio was decreased in AD (see boxed pathways in FIG. 1). These data (i.e., the ratios of the metabolites) indicated that the cysteine and methionine pathway was down-regulated in AD while the tryptophan pathway was up-regulated in AD. There were significant differences in the levels of several compounds of unknown chemical structure between AD and CN (Table 3).

TABLE 3
Metabolic Differences Among Diagnostic Groups

| Groups | Metabolites | Mean | s.d. | Mean | s.d. | P-value | q-value |
|---|---|---|---|---|---|---|---|
| | | AD | | CN | | | |
| AD vs CN | 15-65.533 | 50.85 | 14.72 | 29.52 | 6.54 | <0.001 | <0.001 |
| | 12-94.5 | 100.88 | 10.52 | 85.7 | 32.1 | <0.001 | <0.001 |
| | 8-93.65 | 92.42 | 15.16 | 82.07 | 16.08 | <0.001 | <0.001 |
| | 8-89.433 | 116.74 | 60.38 | 70.45 | 57.71 | <0.001 | <0.001 |
| | 14-64.275 | 51.93 | 18.86 | 33.98 | 17.6 | <0.001 | <0.001 |
| | MET | 74.59 | 24.29 | 51.58 | 26.22 | <0.001 | <0.001 |
| | 9-20.858 | 133.69 | 105.57 | 39 | 46.53 | <0.001 | <0.001 |
| | 5-HIAA | 95.72 | 45.43 | 64.08 | 18.52 | <0.001 | <0.001 |
| | GSH/MET | 1.58 | 0.65 | 2.04 | 0.63 | <0.001 | <0.001 |
| | VMA | 133.63 | 37.32 | 100.66 | 53.56 | <0.001 | 0.001 |
| | 9-29.925 | 174.55 | 95.56 | 126.62 | 89.35 | <0.001 | 0.002 |
| | 5-HIAA/5-HTP | 0.78 | 0.48 | 0.61 | 0.44 | <0.001 | 0.004 |
| | 8-14.983 | 118.51 | 61.42 | 77.78 | 42.98 | 0.002 | 0.008 |
| | 5-40.292 | 20.68 | 16.33 | 23.07 | 14.17 | 0.002 | 0.009 |
| | 13-18.475 | 89.17 | 52.63 | 54.72 | 37.63 | 0.003 | 0.01 |
| | XANTH | 68.92 | 21.18 | 57.21 | 11.68 | 0.004 | 0.02 |
| | GSH | 105.85 | 22.64 | 93.45 | 19.4 | 0.01 | 0.03 |
| | 8-63.675 | 128.69 | 59.77 | 163.5 | 55.61 | 0.01 | 0.03 |
| | | MCI | | CN | | | |
| MCI vs CN | 15-68.542 | 54.08 | 38.65 | 25.1 | 14.34 | <0.001 | <0.001 |
| | 15-65.533 | 63.49 | 62.75 | 29.52 | 6.54 | <0.001 | <0.001 |
| | 14-64.275 | 57.61 | 35.93 | 33.98 | 17.6 | <0.001 | <0.001 |
| | 8-93.65 | 85.14 | 11.28 | 82.07 | 16.08 | <0.001 | <0.001 |
| | 12-94.5 | 88.07 | 10.48 | 85.7 | 32.1 | <0.001 | <0.001 |
| | 5-HIAA/5-HTP | 0.86 | 0.38 | 0.61 | 0.44 | <0.001 | <0.001 |
| | 5-40.292 | 20.09 | 19.43 | 23.07 | 14.17 | <0.001 | 0.002 |
| | GSH/MET | 1.56 | 0.54 | 2.04 | 0.63 | <0.001 | 0.002 |
| | 4-22.117 | 79.45 | 39.24 | 61.83 | 18.44 | <0.001 | 0.004 |
| | 5-HIAA | 83.17 | 28.71 | 64.08 | 18.52 | 0.001 | 0.006 |
| | 13-18.475 | 85.94 | 47.56 | 54.72 | 37.63 | 0.003 | 0.01 |
| | 8-3.675 | 123.82 | 60.32 | 163.5 | 55.61 | 0.003 | 0.01 |
| | URIC/XAN | 0.9 | 0.42 | 0.7 | 0.42 | 0.005 | 0.02 |
| | 5-HTP/TRP | 1.32 | 0.97 | 1.5 | 0.56 | 0.005 | 0.02 |
| | MET | 66.45 | 31.69 | 51.58 | 26.22 | 0.007 | 0.02 |
| | 15-77.017 | 2422.89 | 7373.11 | 742.51 | 2045.72 | 0.007 | 0.02 |
| | KYN/TRP | 0.98 | 0.32 | 0.8 | 0.27 | 0.007 | 0.02 |
| | 8-89.433 | 76.48 | 34.97 | 70.45 | 57.71 | 0.01 | 0.02 |
| | XAN/HX | 1.98 | 0.9 | 3.91 | 3.91 | 0.01 | 0.02 |
| | I-3-AA/TRP | 1.54 | 0.82 | 1.14 | 0.65 | 0.01 | 0.02 |
| | HX | 61.04 | 43.12 | 41.05 | 25.34 | 0.01 | 0.02 |
| | I-3-AA | 139.11 | 79.1 | 98.4 | 63.29 | 0.02 | 0.03 |
| | URIC | 80.45 | 31.44 | 65.24 | 33.9 | 0.02 | 0.03 |
| | 5-HTP | 116.53 | 87.98 | 128.31 | 48.94 | 0.02 | 0.03 |
| | KYN | 91.15 | 42.55 | 69.37 | 26.35 | 0.02 | 0.03 |
| | 15-90.6 | 97.27 | 66.04 | 74.8 | 26.01 | 0.03 | 0.05 |
| | XAN/XANTH | 2.19 | 2.71 | 1.76 | 0.56 | 0.03 | 0.05 |

AD, Alzheimer's Disease; CN, normal cognition; MCI, mild cognitive impairment. Significance cutoff: q-value < 0.05.

Metabolites and Pathways Affected in MCI.

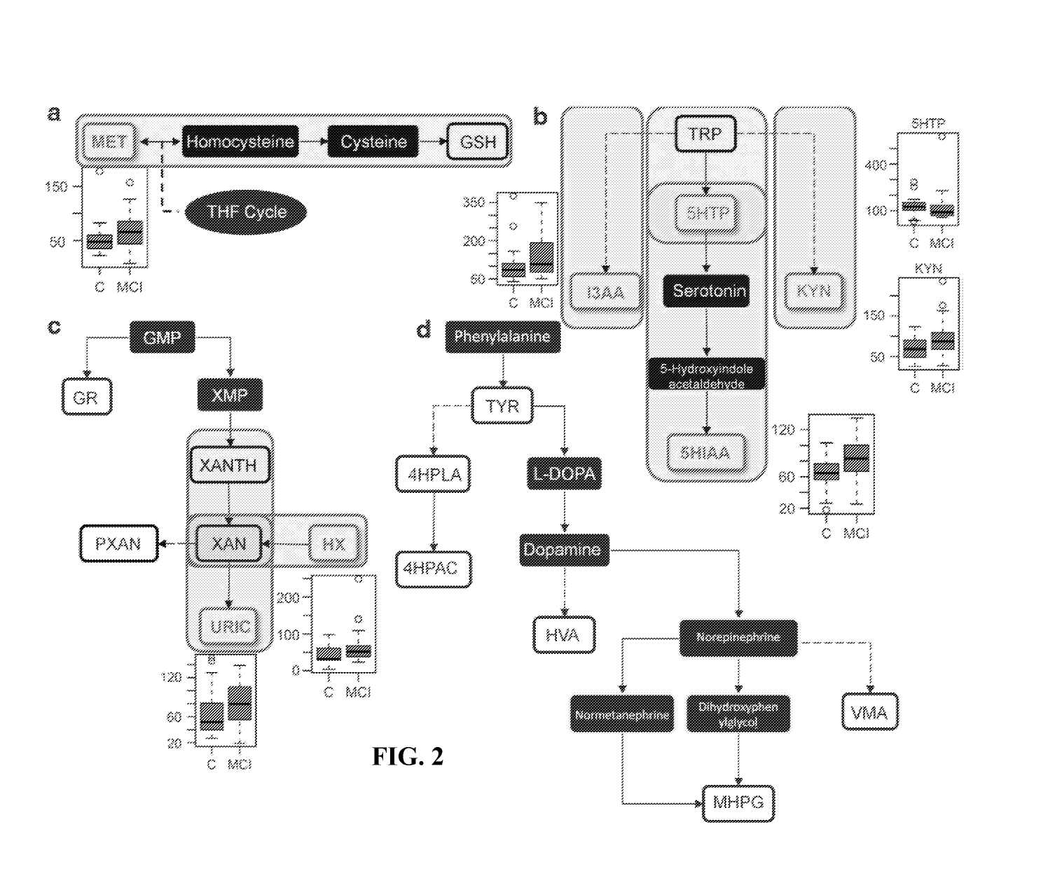

Metabolites that increased in MCI included 5-HIAA, MET, hypoxanthine (HX), indole-3-acetic acid (I-3-AA), uric acid (URIC) and kynurenine (KYN) whereas tryptophan (TRP) was decreased (Table 3 and FIG. 2). In FIG. 2, metabolites in black boxes or circles (with white lettering) were not measured. Similar to AD, the 5-HIAA/5-HTP ratio was increased and GSH/MET ratio was decreased in MCI. Additionally, the ratios of URIC/XAN (Xanthine), KYN/TRP, I-3-AA/TRP and XAN/XANTH were increased and of 5-HTP/TRP and XAN/HX were decreased. These data (i.e., the ratios of metabolites) indicated that cysteine and methionine pathway was down-regulated in MCI while different portions of the tryptophan and purine pathway were up- and down-regulated.

Similar to AD, several compounds of unknown chemical structure were different between MCI and controls (Table 3). Many significant unknown metabolites increased in MCI were those noted in AD.

In the MCI versus AD comparison, 5-HTP was lower in MCI compared with AD. Several unknown metabolites differed between MCI and AD also (Table 4).

TABLE 4
Metabolic Differences between AD and MCI Groups

| | Mean | S.D. | Mean | S.D. | | |
|---|---|---|---|---|---|---|
| Metabolites | AD | | MCI | | p-value | q-value |
| 12.94.5 | 100.88 | 10.52 | 88.07 | 10.48 | 4.1E−10 | 1.9E−08 |
| 8.93.65 | 92.42 | 15.16 | 85.14 | 11.28 | 7.8E−07 | 1.8E−05 |
| 8.89.433 | 116.74 | 60.38 | 76.48 | 34.97 | 1.2E−04 | 1.9E−03 |
| 9.29.925 | 174.55 | 95.56 | 116.85 | 37.40 | 3.5E−04 | 4.1E−03 |
| 8.14.983 | 118.51 | 61.42 | 80.56 | 43.00 | 2.8E−03 | 2.2E−02 |
| 5-HTP | 134.63 | 50.26 | 116.53 | 87.98 | 3.7E−03 | 2.5E−02 |

Metabolite Intercorrelations.

To gain insights into possible structure and/or functions of unknown metabolites changed in AD and MCI, the possible associations of these metabolites with the known metabolites were analyzed (Table 5). Levels of several unknown metabolites that significantly changed in AD and MCI versus controls correlated with levels of known compounds significantly changed in AD and MCI, suggesting that these unknown metabolites may be either structurally or functionally related to the metabolites from one-carbon metabolism and from tyrosine, TRP, and purine pathways.

TABLE 5
Correlations between Unknown Metabolites Changed
in AD and MCI and Known Metabolites in All Participants

| | | Correlation | |
|---|---|---|---|
| Unknown Metabolites | Known Metabolites | Coefficient | P-value |
| 11-51.158 | GR | 0.5 | <0.001 |
| | 4-HPAC | 0.42 | <0.001 |
| | XAN | 0.29 | 0.002 |
| | PXAN | 0.26 | 0.004 |
| | VMA | 0.25 | 0.008 |
| | 4-HPLA | 0.24 | 0.01 |
| | TYR | 0.24 | 0.01 |
| | HVA | 0.23 | 0.01 |
| | GSH | 0.22 | 0.02 |
| 12-94.5 | MET | 0.54 | <0.001 |
| | TRP | 0.26 | 0.005 |
| | 4-HBAC | 0.24 | 0.01 |
| | VMA | 0.23 | 0.01 |
| | TYR | 0.23 | 0.01 |
| | GSH | 0.23 | 0.02 |
| | 5-HIAA | 0.2 | 0.03 |
| 13-18.475 | VMA | 0.34 | <0.001 |
| | MET | 0.22 | 0.02 |
| | HX | 0.21 | 0.02 |
| | 5-HIAA | 0.21 | 0.02 |
| 13-38.49 | TYR | 0.54 | <0.001 |
| | TRP | 0.4 | <0.001 |
| | UA | 0.35 | <0.001 |
| | I3PA | 0.3 | <0.001 |
| | GR | 0.29 | 0.002 |
| | 5-HTP | 0.26 | 0.006 |
| | HX | −0.23 | 0.01 |
| 13-38.49 | KYN | 0.23 | 0.02 |
| | PXAN | 0.22 | 0.02 |
| 14-22.758 | ANT | 0.34 | <0.001 |
| | TYR | 0.31 | <0.001 |
| | TRP | 0.29 | 0.002 |
| | 4-HPAC | 0.26 | 0.004 |
| | MET | 0.26 | 0.005 |
| | GSH | 0.25 | 0.008 |
| | UA | 0.2 | 0.03 |
| 14-75-608 | 5-HTP | 0.33 | <0.001 |
| | GSH | −0.26 | 0.006 |
| | PXAN | 0.22 | 0.02 |
| | MET | −0.21 | 0.03 |
| 15-65-533 | I3PA | 0.54 | <0.001 |
| | MET | 0.44 | <0.001 |
| | KYN | 0.36 | <0.001 |
| | I3AA | 0.26 | 0.005 |
| | GR | 0.24 | 0.009 |
| 15-77.017 | I3PA | 0.47 | <0.001 |
| 5-102.808 | TRP | 0.37 | <0.001 |
| | TYR | 0.31 | <0.001 |
| | MET | 0.31 | <0.001 |
| | KYN | 0.29 | 0.002 |
| | 2-HPAC | 0.28 | 0.003 |
| | UA | 0.27 | 0.003 |
| | 4-HPLA | 0.26 | 0.005 |
| | VMA | 0.25 | 0.008 |
| 8-89.433 | MET | 0.38 | <0.001 |
| 8-93.65 | MET | 0.6 | <0.001 |
| | GSH | 0.38 | <0.001 |
| | 5-HIAA | 0.34 | <0.001 |
| | TYR | 0.32 | <0.001 |
| | TRP | 0.31 | <0.001 |
| | 4-HBAC | 0.29 | 0.002 |
| | VMA | 0.21 | 0.03 |

Significance cutoff: q-value <0.05;
AD, Alzheimer's disease;
MCI, Mild cognitive impairment.

### Example 3

### PLD-DA Models for Categorical Separation of AD, MCI, and CN

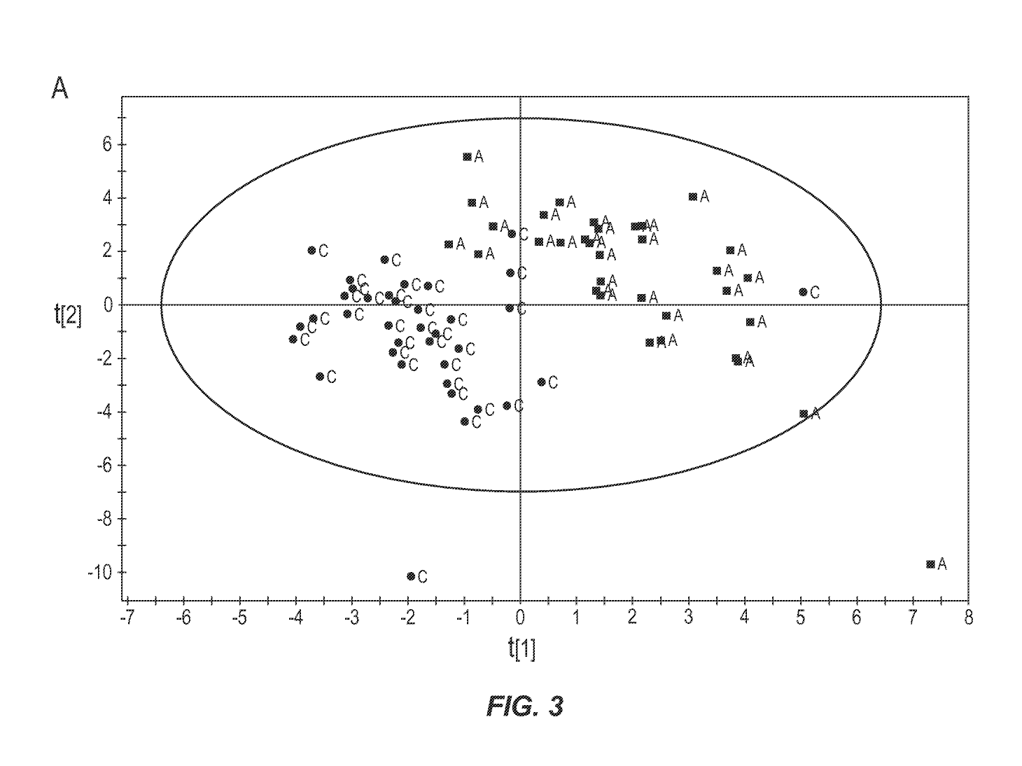

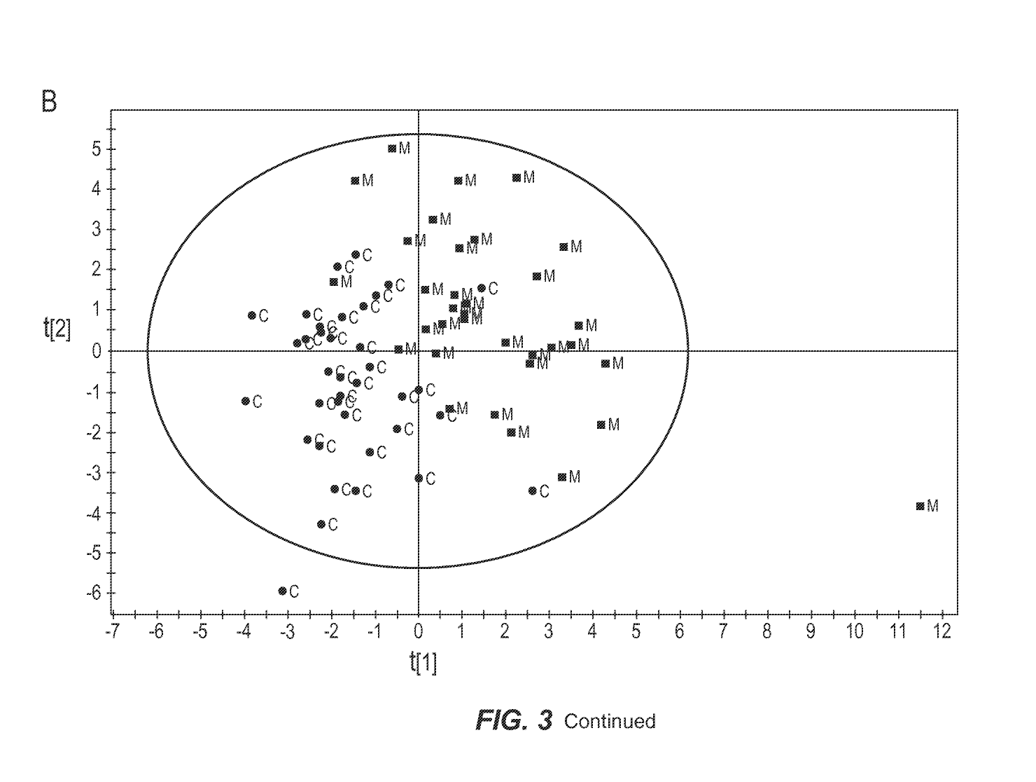

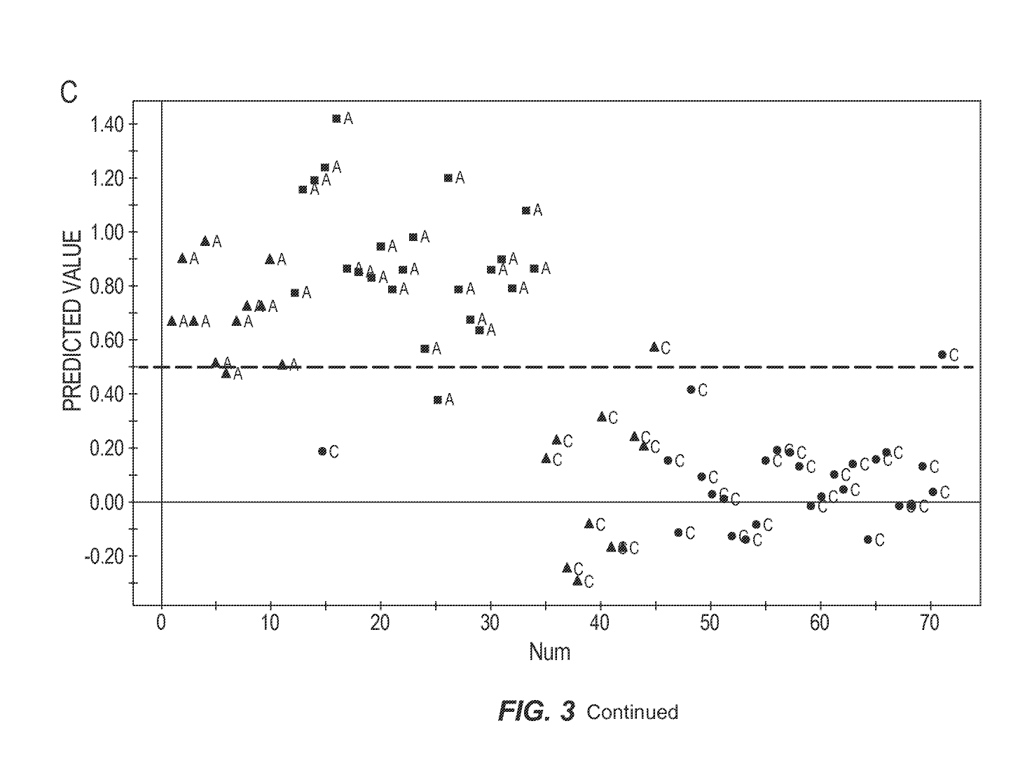

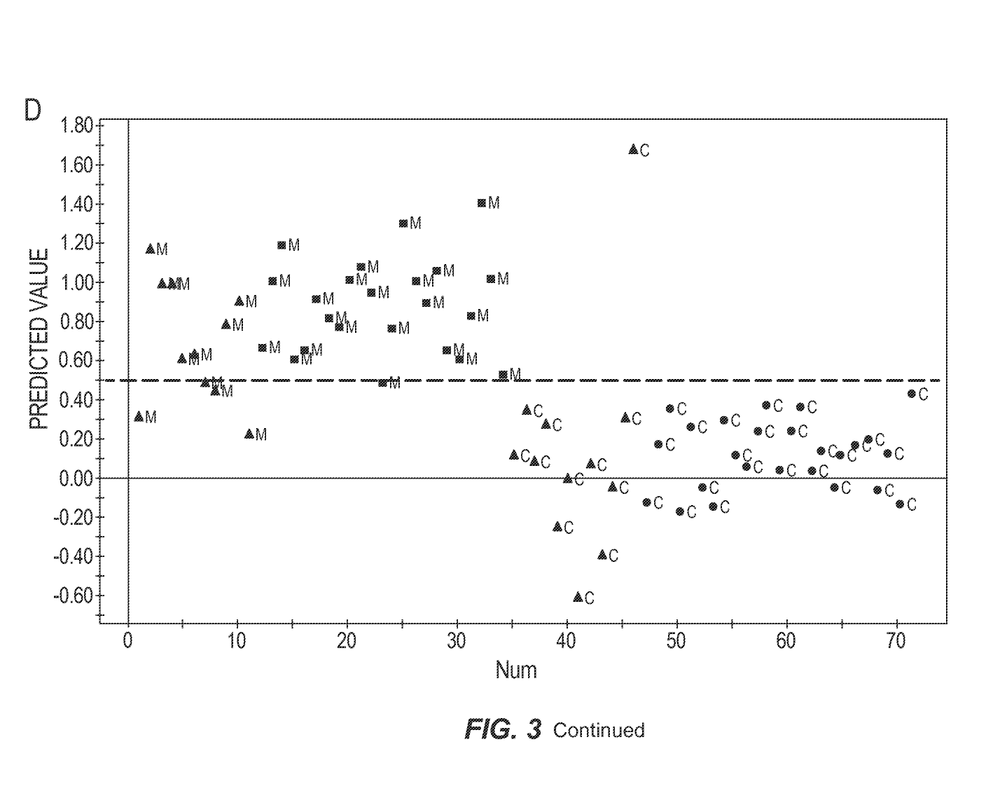

The value of metabolic profiles in separating disease participants and controls was evaluated. PLS-DA models were constructed for each pair of disease status (AD vs CN and MCI vs CN). The performance of the models was evaluated by cross-validation using correct classification rate together with sensitivity and specificity. The correct classification rate for AD versus CN was 83.1% (sensitivity: 76.5% and specificity: 89.2%). The correct classification rate for MCI versus CN was also 83.1% (sensitivity: 73.5% and specificity: 91.9%). FIG. 3 shows the classification results using a two-component PLS-DA model, with corresponding variable importance in projection scores provided in Table 6.

TABLE 6
Variable Importance in Projection (VIP) Values for
PLS-DA Models Discriminating between
Different Groups of Participants

| | |
|---|---|
| AD vs CN | MCI vs CN |

| Metabolites | VIP | Metabolites | VIP |
|---|---|---|---|
| 15-65.533 | 2.71 | 15-68.542 | 1.92 |
| 9-20.858 | 1.95 | 14-64.275 | 1.70 |
| 15-30.5 | 1.74 | 15-65.533 | 1.66 |
| 14-64.275 | 1.70 | 13-18.475 | 1.63 |
| 5-HIAA | 1.57 | 9-20.858 | 1.63 |
| 8-63.675 | 1.56 | 8-63.675 | 1.58 |
| 13-18.475 | 1.50 | 5-HIAA | 1.57 |
| 8-89.433 | 1.49 | 13-78.992 | 1.51 |
| 12-94.5 | 1.48 | 14-45.642 | 1.49 |
| 8-93.65 | 1.39 | 4-22.117 | 1.45 |
| 8-14.983 | 1.34 | 15-30.5 | 1.42 |
| 9-17.817 | 1.31 | HX/XAN | 1.38 |
| 14-22.758 | 1.28 | 9-17.817 | 1.32 |
| 9-29.925 | 1.22 | 8-28.508 | 1.30 |
| 12-34.975 | 1.22 | 8-72.05 | 1.29 |
| 13-38.49 | 1.21 | 6-10.383 | 1.22 |
| HVA/5-HIAA | 1.19 | 12-50.183 | 1.19 |
| 14-45.642 | 1.16 | 14-75.608 | 1.15 |
| 15-90.6 | 1.15 | 15-15.567 | 1.14 |
| 11-59.092 | 1.13 | HVA/5-HIAA | 1.08 |
| 13-92.333 | 1.13 | 13-92.333 | 1.08 |
| 13-21 | 1.11 | 13-44.608 | 1.07 |
| 15-26.05 | 1.09 | 12-34.975 | 1.06 |
| 11-47.908 | 1.03 | 11-77.808 | 1.06 |
| 13-78.992 | 1.03 | 12-52.75 | 1.06 |
| 4-22.117 | 1.03 | 15-26.05 | 1.06 |
| 5-102.808 | 1.00 | TYR/4-HPLA | 1.05 |
| | | 11-36.75 | 1.05 |
| | | TRP/KYN | 1.02 |
| | | 11-59.092 | 1.00 |

AD, Alzheimer's disease;
CN, Normal cognition;
MCI, Mild cognitive impairment;
PLS-DA, partial least square-discriminant analysis.

### Example 4

### Correlation Between Metabolites, Proteins, and MMSE Scores

A pair-wise correlation analysis revealed significant associations between metabolites and each of Ab42, t-tau and p-tau (Table 7).

TABLE 7
Correlations of Metabolites with Proteins in All Participants

| Proteins | Metabolites | Correlation coefficient | P-value | q-value |
|---|---|---|---|---|
| Ab42 | 11-46.55 | −0.36 | <0.001 | 0.006 |
| | MET | −0.33 | <0.001 | 0.008 |
| | 11-36.75 | −0.33 | <0.001 | 0.008 |
| | 13-18.475 | −0.32 | <0.001 | 0.008 |
| | 15-65.533 | −0.28 | 0.004 | 0.03 |
| | VMA | −0.28 | 0.005 | 0.03 |
| | 9-20.858 | −0.27 | 0.005 | 0.03 |
| | 5-102.808 | −0.26 | 0.008 | 0.03 |
| | GSH | −0.25 | 0.01 | 0.04 |
| p-tau | 13-44.608 | 0.44 | <0.001 | <0.001 |
| | 12-41.200 | 0.36 | <0.001 | 0.002 |
| | XAN | 0.36 | <0.001 | 0.002 |
| | VMA | 0.29 | 0.002 | 0.01 |
| | 11-46.55 | 0.31 | 0.002 | 0.01 |
| | 11-60.917 | 0.3 | 0.002 | 0.01 |
| | XANTH | 0.28 | 0.004 | 0.02 |
| | 4-HPLA | 0.27 | 0.006 | 0.02 |
| | HVA | 0.26 | 0.007 | 0.02 |
| | GSH | 0.26 | 0.007 | 0.02 |
| | 13-74.392 | 0.26 | 0.008 | 0.02 |
| | 9-20.858 | 0.25 | 0.01 | 0.02 |
| | 14-22.758 | −0.25 | 0.01 | 0.02 |
| | 9-29.34 | 0.25 | 0.01 | 0.02 |
| | 9-29.925 | 0.24 | 0.01 | 0.02 |
| | 14-34.25 | 0.22 | 0.03 | 0.04 |
| t-tau | 13-44.608 | 0.59 | <0.001 | <0.001 |
| | XAN | 0.44 | <0.001 | <0.001 |
| | 12-41.200 | 0.41 | <0.001 | <0.001 |
| | 11-46.55 | 0.39 | <0.001 | <0.001 |
| | 13-78.992 | 0.35 | <0.001 | 0.003 |
| | 4-HPLA | 0.33 | <0.001 | 0.006 |
| | 9-29.925 | 0.33 | <0.001 | 0.007 |
| | 5-HIAA | 0.32 | <0.001 | 0.009 |
| | 9-20.858 | 0.31 | 0.001 | 0.01 |
| | VMA | 0.3 | 0.002 | 0.02 |
| | 8-14.983 | 0.28 | 0.004 | 0.03 |
| | GSH | 0.27 | 0.005 | 0.03 |
| | 14-34.25 | 0.26 | 0.007 | 0.04 |
| | URIC | 0.25 | 0.01 | 0.04 |
| | 2-HPAC | 0.25 | 0.01 | 0.04 |
| | XANTH | 0.25 | 0.01 | 0.04 |
| | 9-25.825 | 0.25 | 0.009 | 0.04 |
| | 11-60.917 | 0.26 | 0.008 | 0.04 |
| | 9-29.34 | 0.25 | 0.01 | 0.04 |

Ab42, amyloid-β;
p-tau, phosphorylated tau;
t-tau, total tau.
Significance cutoff: q-value <0.05.

Correlations between MET, VMA and Ab42; between XAN, 4-hydroxyphenyllactic acid (4-HPLA), 5-HIAA, VMA, GSH, (2-hydroxyphenylacetic acid) and t-tau; and between XAN, VMA, 4-HPLA, HVA, GSH, XANTH and p-tau were found. For correlations within each group, see Table 8.

TABLE 8
Correlations of Metabolites with Proteins in Each Diagnostic Group

| | AD | | | | MCI | | | | CN | | | |
|---|---|---|---|---|---|---|---|---|---|---|---|---|
| | | Correlation | | | | Correlation | | | | Correlation | | |
| Protein | Metabolite | coefficient | P-value | q-value | Metabolite | coefficient | P-value | q-value | Metabolite | coefficient | P-value | q-value |
| Ab42 | | | | | | | | | 11-36.75 | −0.53 | <0.001 | 0.05 |
| p-tau | | | | | 14-34.25 | 0.65 | <0.001 | 0.002 | | | | |
| | | | | | 13-44.608 | 0.59 | <0.001 | 0.006 | | | | |
| | 13-44.608 | 0.68 | <0.001 | <0.001 | 14-34.25 | 0.72 | <0.001 | <0.001 | | | | |
| | XAN | 0.66 | <0.001 | <0.001 | 13-44.608 | 0.64 | <0.001 | 0.001 | | | | |
| | 11-46.55 | 0.59 | <0.001 | 0.003 | | | | | | | | |
| | 13-78.992 | 0.51 | 0.002 | 0.02 | | | | | | | | |
| t-tau | 12-41.200 | 0.52 | 0.002 | 0.02 | | | | | | | | |
| | 9-29.34 | 0.53 | 0.001 | 0.02 | | | | | | | | |
| | 4-HPLA | 0.47 | 0.005 | 0.03 | | | | | | | | |
| | 4-HPAC | 0.47 | 0.005 | 0.03 | | | | | | | | |
| | VMA | 0.44 | 0.009 | 0.05 | | | | | | | | |

AB42, amyloid-β; AD, Alzheimer's Disease; CN, normal cognition; MCI, mild cognitive impairment; p-tau, phosphorylated taul; t-tau, total tau. Significance cutoff: q-value < 0.05.

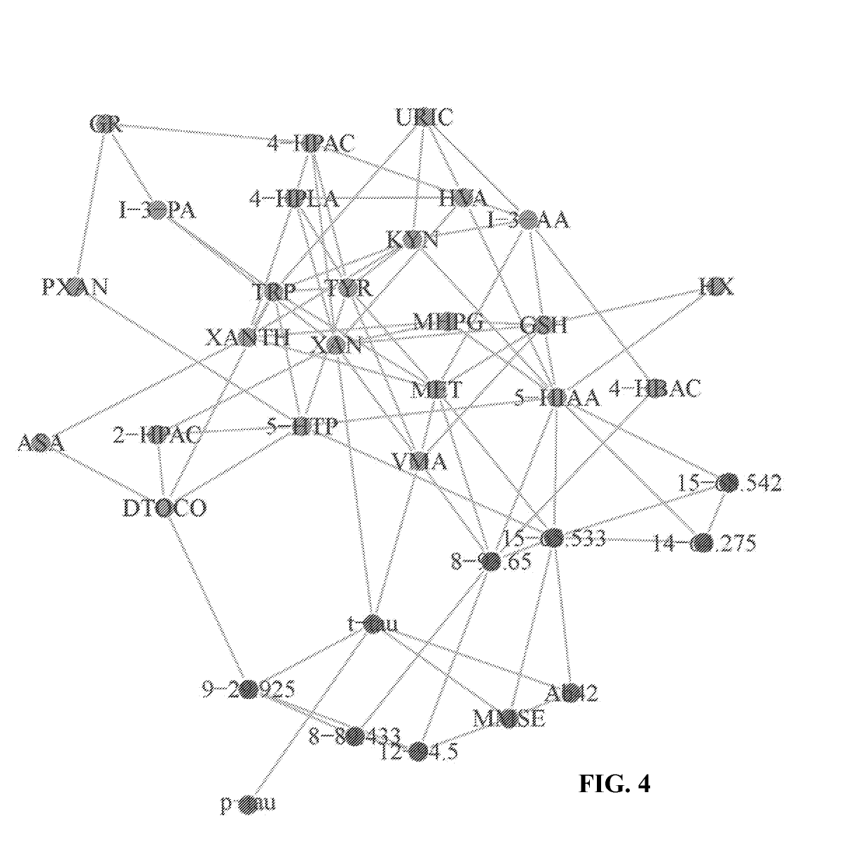

A partial correlation network was built among protein AD biomarkers, MMSE, all known metabolites and seven unknown metabolites to be related to disease status (FIG. 4). Two variables are connected within the network if their mutual correlation cannot be fully mediated by the other variables. The false discovery rate was controlled at 0.05. T-tau is directly related to VMA, XAN and 9-29.925, Ab42 is related to 15-65.533, and MMSE is related to 15-65.533 and 12-94.5. The unknown metabolite 15-65.533 was related to MET and 5-HIAA, the two metabolites altered in AD CSF.

In summary, the above data in Examples 2-4 demonstrated that the levels of overlapping groups of metabolites (but not the same) were altered in Alzheimer's Disease and mild cognitive impairment. These altered levels in cerebrospinal fluid indicated perturbations in the cysteine and methionine pathway, tryptophan pathway, tyrosine pathway, and purine pathway in patients with Alzheimer's Disease and mild cognitive impairment.

These data additionally demonstrated that in both Alzheimer's Disease patients and mild cognitive impairment patients, the levels of methionine (the precursor of homocysteine) were increased, but the ratio of methionine: glutathione was decreased. This result indicated that glutathione depletion in Alzheimer's Disease patients resulted from perturbations in the cysteine and methionine pathway at the level of synthesis of glutathione from cysteine.

Additionally, the above data indicated that VMA levels were increased in lumbar cerebrospinal fluid from patients with Alzheimer's Disease. VMA levels did not differ between Alzheimer's Disease patients receiving memantine and Alzheimer's Disease patients not receiving memantine (data not shown), thereby indicating that elevated VMA levels were not the result of medication. VMA levels were also highest amongst ε4/ε4 participants as compared to ε3/ε4 and non-ApoE participants (data not shown).

The above data also indicated that 5-HIAA levels were increased in Alzheimer's Disease patients and mild cognitive impairment patients. No correlation was found between use of medications in these patients and 5-HIAA levels (data not shown), thereby indicating that the elevated 5-HIAA levels were not the result of medication.

Lastly, the above partial correlation network further indicated links between proteins implicated in Alzheimer's Disease and metabolites. For example, the correlation of t-tau to VMA and XAN indicated that the norepinephrine pathway and purine pathway may be involved in t-tau pathology. The unidentified compound 15-65.533 may link the cysteine and methionine pathway and methylation to amyloid-beta pathology.

### Example 5

### Materials and Methods for Examples 6-8

Participants.

Metabolomic, protein and genetic data for this study were gathered from a cross-section of participants who were recruited and evaluated in clinical research by the Penn Memory Center. Most of these participants were enrolled in a prospective multi-site longitudinal biomarker study and were also included in the above study (i.e., Examples 1-5) focusing on LC-ECA metabolites. Forty AD patients and 38 controls with banked CSF samples were analyzed. Written informed consent was collected as appropriate.

Inclusion and Exclusion Criteria.

For the AD subgroup, subjects had to meet National Institute of Neurological, Communicative Disorders and Stroke-Alzheimer Disease and Related Disorders Association criteria for probable or possible AD. All but one patient were classified as having mild to moderate dementia based on combination of clinical judgment, Mini Mental State Exam (MMSE) score and Functional Rating Scale (FRS) score. Participants could be on stable approved therapies. Participants were excluded from this group if they had a history of clinically meaningful stroke, Parkinson's disease, untreated current major depression, psychosis or a primary diagnosis of a non-AD dementia.

To be included in the cognitively normal subgroup, the following inclusion criteria had to be met: 1) No significant cognitive impairment verified by psychometric testing norms, and 2) No significant change in functional abilities verified by a knowledgeable informant. Participants were excluded if they had a history of significant stroke, current untreated major depression, psychosis, mild cognitive impairment (MCI) or dementia. Subjects in both groups had to be over 65, have a reliable informant and consent to longitudinal follow up.

Diagnostic assessments were generally made in a consensus conference after comprehensive neurologic, physical and neuropsychological testing was performed. Most patients had multiple psychometric tests, including the Clinical Dementia Rating, the Dementia Rating Scale-Second Edition (DRS-2), the MMSE, and tests of frontal executive function, memory, language, praxis, visuo-spatial construction, motor performance, mood and function. MMSE scores were not always available at the time of baseline blood collection but the nearest available MMSE was used for staging purposes along with function and clinical judgment.

CSF Collection.

Baseline CSF samples obtained in polypropylene tubes were utilized for metabolomics. CSF was obtained by lumbar puncture using an atraumatic Sprotte needle in most cases. To minimize contamination from blood associated with needle insertion, the first 1-2 ml of CSF (or more if needed) were discarded and the next 20 ml were aliquoted into 0.5 ml portions, bar coded and stored in a −80° C. freezer until processing. The standardized Luminex multiplex assay technique for amyloid beta 1-42 (Ab42), total tau (t-tau) and tau phosphorylated at the threonine 181 position (p-tau) was used in this study. Aliquots were shipped overnight on dry ice for metabolomics processing.

Metabolomics Profiling: LC-ECA.

The LC-ECA method was specific for compounds that underwent LC-ECA oxidation or reduction, and included multiple compounds from the tyrosine, tryptophan, sulfur amino acid and purine pathways, as well as markers of oxidative stress and protection (see Table 9).

TABLE 9
List of Known Compounds Quantified by the LC-ECA Platform

| Metabolite by Pathways | Abbreviation |
|---|---|
| Tryptophan | |
| Tryptophan | TRP |
| 5-Hydroxyindoleacetic acid | 5-HIAA |
| 5-Hydroxytryptophan | 5-HTP |
| Kynurenine | KYN |
| Indole-3-acetic acid | I-3-AA |
| Tyrosine | |
| 4-Hydroxyphenylacetic acid | 4-HPAC |
| Homovanillic acid | HVA |
| Methoxyhydroxyphenlyglycol | MHPG |
| Tyrosine | TYR |
| Vanillylmandelic acid | VMA |
| Phenylalanine | |
| 4-Hydroxybenzoic acid | 4-HBAC |
| 4-Hydroxyphenyllactic acid | 4-HPLA |
| 2-Hydroxyphenylacetic acid | 2-HPAC |
| Purine | |
| Guanosine | GR |
| Hypoxanthine | HX |
| Uric acid | URIC |
| Xanthine | XAN |
| Xanthosine | XANTH |
| Paraxanthine | PXAN |
| Cysteine and Methionine | |
| Glutathione (reduced) | GSH |
| Methionine | MET |
| Other | |
| Ascorbic acid | ASA |
| Delta-tocopherol | DTOCO |
| Indole-3-propionic acid | I-3-PA |

At the time of preparation, a pool was created from equal amounts of small aliquots of each study sample, which was treated identically to a sample. The pooled samples were run after every six study samples, followed by a known standards mix to ensure uniformity along the length of the run. Metabolite peak identification was carried out using the CEAS software (ESA, Inc., Chelmsford, Mass.). The main metabolite peaks of known and unknown compounds were aligned and relative concentrations to a central CSF sample pool (taken at 100%) were measured. These peak-tables were used for the subsequent statistical analysis, which focused on 71 total metabolites, of which 24 were known compounds (Table 9).

GC-TOF Mass Spectrometry.

CSF samples were aliquoted and maintained at −80° C. until use, at which point 30 μl of CSF samples were thawed, extracted and derivatized. Briefly, 15 μl aliquots were extracted with 1 ml of degassed acetonitrile:isopropanol:water (3:3:2) at −20° C., centrifuged and decanted with subsequent evaporation of the solvent to complete dryness. A clean-up step with acetonitrile/water (1:1) removed membrane lipids and triglycerides, and the supernatant was again dried down. Internal standards C8-C30 fatty acid methyl esters were added and the sample was derivatized with methoxyamine hydrochloride in pyridine and subsequently by N-Methyl-N-(trimethylsilyl)trifluoroacetamide (MSTFA) (Sigma-Aldrich) for trimethylsilylation of acidic protons.

A Gerstel MPS2 automatic liner exchange system was used to inject 1 μl of sample at 50° C. (ramped to 250° C.) in splitless mode with a 25-second splitless time. An Agilent 6890 gas chromatograph (Santa Clara, Calif.) was used with a 30 m long, 0.25 mm i.d. Rtx5Sil-MS column with 0.25 μm 5% diphenyl film; an additional 10 m integrated guard column was used (Restek, Bellefonte Pa.). Chromatography was performed at a constant flow of 1 ml/minute, ramping the oven temperature from 50° C. to 330° C. over 22 minutes. Mass spectrometry used a Leco Pegasus IV time of flight mass spectrometer with a 280° C. transfer line temperature, electron ionization at −70 V and an ion source temperature of 250° C. Mass spectra were acquired from m/z 85-500 at 20 spectra/second and 1750 V detector voltage.

Result files were exported to servers and further processed by metabolomics BinBase database. All database entries in BinBase were matched against the Fiaehn mass spectral library of 1,200 authentic metabolite spectra using retention index and mass spectrum information or the NIST05 commercial library. Identified metabolites were reported if present with at least 50% of the samples per study design group (as defined in the SetupX database). Quantitative data were normalized to the sum intensities of all known metabolites and used for statistical investigation. Data on a total of 299 metabolites were collected using the MS platform.

Statistical Methods.

Statistical analysis was performed in two stages to find variables that discriminate between AD participants and controls. First, univariate analyses were performed to understand the potential associations between the covariates collected and the disease. The use of AD treatment drugs (cholinesterase inhibitors and memantine), as well as antidepressants, antipsychotics, anxiolytics, corticosteroids and statins were investigated to identify metabolites that might potentially be associated with drug metabolism and/or response. Second, multivariate modeling was performed using different combinations of data types (metabolomic, proteins, etc.) to evaluate the potential discriminatory power of each of these data types alone and in combination for predicting AD. To evaluate the predictive potential of these variables, the models were evaluated using cross-validation to assess the predictive performance of the models and further refine the variable included in a prediction analysis, so that the resulting models were evaluated on the testing data, as opposed to the training data.

Univariate Analysis.

Fisher's exact tests were performed to examine the potential association of gender, race, and the use of different classes of drugs with disease status; two-sample t tests were used to test the difference in education, age and MMSE score between the two diagnostic groups.

Model Building to Evaluate the Discrimination Potential of Variables.

To evaluate the discriminatory power of the metabolites and compare to the Luminex values, predictive models of AD vs. control status were built. Prior to model-building analysis, the raw metabolite values were visually inspected by quantile-quantile normal plots to assess normality. Metabolites were log-transformed to improve normality. Metabolites were also filtered to prevent any potential confounding with the drug therapies used to treat the disease. This was done because different drugs are used in AD participants than in controls, and it was expected that metabolite profiles could change in response to treatment. Then, for the nominally significantly associated drugs, all metabolites were tested for association with drug status/use using Kruskal-Wallis tests. Those metabolites that were even nominally associated with drug status (p<0.05) were filtered out prior to model building as they may potentially be related to drug metabolism and/or response. While this may be overly conservative, this provided certainty that any potential discrimination gained by adding the metabolites into a model was not confounded by drug response/metabolism. Additionally, metabolites were tested for association with both the ApoE genotype, with genotype coded as high risk and low risk groups (where E3/E4 and E4/E4 genotypes were high risk and all others were considered low risk) using Kruskal-Wallis tests of association. Again, nominally associated metabolites were removed prior to model building to prevent confounding with risk genotype.

Once the metabolites were filtered for independence from drug use and genotype, forward step-wise logistic regression was performed using a Bayesian Information Criterion (BIC) for variable selection and modeling with several different sets of variables. First, models were built using each of the following types of variables alone to evaluate the maximal potential prediction from each set of variables: Luminex proteins, LC-ECA metabolites, and GC-TOF metabolites. Second, model building was performed using all possible two-way combinations of variables (e.g., Luminex proteins plus the LC-ECA metabolites, Luminex proteins plus the GC-TOF metabolites, etc.). Next, models were built with all possible three-way combinations of variables. Finally, modeling was performed with all possible predictive variables.

In order to assess the predictive performance of the metabolites (and to limit potential overfitting), the model building was performed using five-fold cross-validation to evaluate the stability of the variable selection and model fit. The step-wise modeling approach was repeated for every ⅘ split of the data, and the variables included in the model were recorded along with a training AUC and a testing AUC (calculated on the ⅕ of the data left out of model building). In each cross-validation interval, the variables included were recorded, as the final model was selected based on cross-validation consistency (picking the variable[s] that were selected in the most cross-validation models). By using cross-validation, a prediction error from the withheld data used in the validation process was estimated. While such an analysis is not as powerful for assessing the true predictive performance of a model, it was well established that k-fold cross-validation provides an estimate of the predictive performance, and k=5 was considered a reasonable compromise between bias and variance for this estimate.

The predictive performance of the resulting models was evaluated using area-under the curve (AUC) values. Because of the high dimensional and sparse nature of the data, to try to assess whether the resulting models were better than would be expected by chance, permutation testing was performed to ascribe statistical significance to the resulting models. One thousand permuted datasets were generated, randomizing the case status at the same proportions as in the real data, and the entire data analysis procedure was repeated. The best correlation coefficient and AUC for each permuted dataset was recorded, and an empirical distribution of model fit statistics was generated across the 1000 permuted datasets. Then the values from the real data analysis were compared to the empirical distribution to generate an empirical p-value.

To test whether there were significant differences in the predictive performances of the resulting models (i.e., whether the differences were just by chance or were likely to represent true differences), DeLong's tests were performed between the different models. A Bonferroni correction for the number of tests performed was used to determine the alpha level for significance for these AUC comparisons.

Finally, Pearson correlation analyses was used to test for correlation of the best metabolites (from the final predictive model) and MMSE scores.

### Example 6

### Demographic Differences

Tests that compared clinical and demographic variables showed the AD and control participants to be generally well matched for age and gender. Among these variables, the only significant differences between groups were a higher educational level for controls (though this association was only nominally significant) and the use of two disease treatment drugs, cholinesterase inhibitor and/or memantine, in a subset of AD participants (Table 10).

TABLE 10
Participant Demographics and Clinical Characteristics
Participant demographics and clinical characteristics

| | AD | CN | | |
|---|---|---|---|---|
| Characteristics | (N = 40) | (N = 38) | p-value | Test |
| Mean Age +/− SD | 69.0 +/− 9.1 | 69.5 +/− 9.7 | 0.825 | T |
| Mean Years of | 14.8 +/− 3.6 | 16.6 +/− 3.0 | 0.015 | T |
| Education +/− SD | | | | |
| Mean MMSE +/− SD | 19.9 +/− 7.7 | 29.2 +/− 1.3 | 1.00E−09 | T |
| % Male | 25 | 34.21 | 0.459 | F |
| % Caucasian | 82.5 | 86.8 | 1 | F |
| % Taking Antidepressant | 30 | 21.05 | 0.441 | F |
| % Taking Antipsychotic | 5 | 0 | 0.494 | F |
| % Taking Anxioytic | 12.5 | 13.2 | 1 | F |
| % Taking Corticosteroids | 2.5 | 13.2 | 0.104 | F |
| % Taking Cholinesterase | 37.5 | 0 | <1.0E−9 | F |
| Inhibitor | | | | |
| % Taking Memantine | 15 | 0 | 0.026 | F |
| % Taking Statins | 20 | 26.3 | 0.595 | F |

AD, Alzheimer's Disease;
CN, Normal Cognition;
SD, Standard Deviation;
T two-sample t test, two sided;
MMSE, Mini-Mental State Exam obtained at date closest to sample if not currently available;
F Fisher's exact test, two sided.

### Example 7

### Drug Associations

The results of the tests of association for the metabolites against drug use resulted in 134 that were nominally associated. Additionally, two metabolites were nominally associated with ApoE genotype status. Metabolites' associations and their p-values from the drug association analysis are listed in Table 11. This analysis was used to correct for effects of medications taken by the subjects in the study.

TABLE 11
Metabolites' Associations and their p-values from the Drug Association Analysis

| | | | K-Wallis P- |
|---|---|---|---|
| Drug | Platform | Metabolite | Value |
| Antipsychotic | GC-TOF | 268306 | 0.000000054 |
| Antipsychotic | GC-TOF | 202599 | 0.00000058 |
| Antipsychotic | LC-ECA | XAN | 0.0000022 |
| Anxioytic | GC-TOF | 268483 | 0.000027 |
| Antipsychotic | LC-ECA | 5_40_292 | 0.00003 |
| Antipsychotic | GC-TOF | 280940 | 0.000046 |
| Antipsychotic | GC-TOF | ARABINOSE | 0.000052 |
| Anxioytic | GC-TOF | 307965 | 0.000081 |
| Anxioytic | GC-TOF | 309545 | 0.0002 |
| Anxioytic | GC-TOF | 309641 | 0.00024 |
| Anxioytic | GC-TOF | 268313 | 0.00026 |
| Anxioytic | GC-TOF | 235414 | 0.00027 |
| Anxioytic | GC-TOF | 306157 | 0.00047 |
| Antidepressant | GC-TOF | 293097 | 0.0005 |
| Statins | LC-ECA | 5HTP | 0.00065 |
| Anxioytic | GC-TOF | 309642 | 0.00078 |
| Anxioytic | GC-TOF | 312679 | 0.0008 |
| Anxioytic | GC-TOF | 309538 | 0.00084 |
| Anxioytic | GC-TOF | 234015 | 0.00087 |
| Antidepressant | GC-TOF | 296108 | 0.0011 |
| Antidepressant | LC-ECA | 8_63_675 | 0.0011 |
| Anxioytic | GC-TOF | 268306 | 0.0012 |
| Anxioytic | LC-ECA | 11_46_55 | 0.0012 |
| Antipsychotic | GC-TOF | 306152 | 0.0013 |
| Anxioytic | GC-TOF | 204425 | 0.0014 |
| Corticosteroids | GC-TOF | 310010 | 0.0014 |
| Anxioytic | LC-ECA | 5_40_292 | 0.0015 |
| Anxioytic | GC-TOF | 268420 | 0.0016 |
| Statins | LC-ECA | 12_50_183 | 0.0016 |
| Statins | LC-ECA | 9_33_1 | 0.0018 |
| Anxioytic | GC-TOF | 211935 | 0.002 |
| Antipsychotic | GC-TOF | 301825 | 0.0021 |
| Corticosteroids | LC-ECA | 4HBAC | 0.0024 |
| Antidepressant | LC-ECA | 13_74_392 | 0.0028 |
| Anxioytic | LC-ECA | 14_36_45 | 0.0028 |
| Anxioytic | GC-TOF | 2_HYDROXYBUTANOIC_ACID | 0.0032 |
| Anxioytic | GC-TOF | 3_HYDROXYPROPIONIC_ACID | 0.0033 |
| Anxioytic | LC-ECA | GR | 0.0033 |
| Anxioytic | GC-TOF | 2_DEOXYTETRONIC_ACID_NIST | 0.0035 |
| Anxioytic | LC-ECA | XAN | 0.0036 |
| Anxioytic | GC-TOF | 215978 | 0.004 |
| Anxioytic | GC-TOF | 301825 | 0.0041 |
| Antipsychotic | GC-TOF | 303060 | 0.0049 |
| Anxioytic | GC-TOF | 202599 | 0.0053 |
| Corticosteroids | GC-TOF | 202885 | 0.0053 |
| Antipsychotic | GC-TOF | ERYTHRONIC_ACID_LACTONE | 0.0055 |
| Antipsychotic | GC-TOF | 215739 | 0.0056 |
| Antidepressant | LC-ECA | MHPG | 0.0058 |
| Anxioytic | LC-ECA | PXAN | 0.0064 |
| Anxioytic | GC-TOF | 269160 | 0.0068 |
| Anxioytic | GC-TOF | 231657 | 0.0069 |
| Corticosteroids | LC-ECA | PXAN | 0.0069 |
| Anxioytic | GC-TOF | 306156 | 0.007 |
| Anxioytic | GC-TOF | 4_HYDROXYBUTYRIC_ACID | 0.0078 |
| Antipsychotic | LC-ECA | 12_41_200 | 0.0082 |
| Anxioytic | GC-TOF | 306159 | 0.0083 |
| Anxioytic | GC-TOF | 309873 | 0.0084 |
| Antipsychotic | GC-TOF | GLUCOSE | 0.0084 |
| Antipsychotic | LC-ECA | 4HBAC | 0.0084 |
| Anxioytic | GC-TOF | 312289 | 0.0095 |
| Corticosteroids | GC-TOF | THREONIC_ACID | 0.0096 |
| Antipsychotic | LC-ECA | 4_32_592 | 0.0098 |
| Antidepressant | GC-TOF | 267816 | 0.012 |
| Anxioytic | GC-TOF | 268579 | 0.012 |
| Antidepressant | GC-TOF | 296106 | 0.012 |
| Antidepressant | GC-TOF | 312977 | 0.012 |
| Anxioytic | GC-TOF | ENOLPYRUVATE_NIST | 0.012 |
| Corticosteroids | GC-TOF | ISOTHREONIC_ACID | 0.012 |
| Antipsychotic | GC-TOF | 312679 | 0.013 |
| Anxioytic | GC-TOF | 231947 | 0.014 |
| Anxioytic | GC-TOF | ACETOPHENONE_NIST | 0.014 |
| Anxioytic | GC-TOF | SALICYLALDEHYDE | 0.015 |
| Antipsychotic | LC-ECA | 14_36_45 | 0.015 |
| Corticosteroids | LC-ECA | 9_19_067 | 0.015 |
| Antidepressant | LC-ECA | 9_25_825 | 0.015 |
| Antidepressant | GC-TOF | 210168 | 0.016 |
| Corticosteroids | GC-TOF | 309873 | 0.016 |
| Corticosteroids | GC-TOF | 312592 | 0.016 |
| Antipsychotic | GC-TOF | ARABITOL | 0.016 |
| Anxioytic | LC-ECA | VMA | 0.017 |
| Anxioytic | LC-ECA | 9_33_1 | 0.017 |
| Anxioytic | GC-TOF | 215494 | 0.018 |
| Anxioytic | GC-TOF | 240432 | 0.018 |
| Anxioytic | GC-TOF | PHOSPHORIC_ACID | 0.018 |
| Antipsychotic | GC-TOF | 224849 | 0.019 |
| Anxioytic | GC-TOF | 228911 | 0.019 |
| Anxioytic | LC-ECA | KYN | 0.019 |
| Antidepressant | LC-ECA | 8_28_508 | 0.019 |
| Statins | LC-ECA | 8_63_675 | 0.019 |
| Corticosteroids | GC-TOF | 199553 | 0.02 |
| Anxioytic | GC-TOF | INOSITOL_ALLO_{—} | 0.02 |
| Anxioytic | GC-TOF | 281907 | 0.021 |
| Statins | GC-TOF | 309573 | 0.021 |
| Corticosteroids | LC-ECA | 13_84_975 | 0.021 |
| Anxioytic | LC-ECA | 5_25_075 | 0.021 |
| Statins | LC-ECA | 8_14_983 | 0.021 |
| Anxioytic | LC-ECA | 8_63_675 | 0.021 |
| Corticosteroids | GC-TOF | 215739 | 0.022 |
| Anxioytic | GC-TOF | 231097 | 0.022 |
| Anxioytic | GC-TOF | 309788 | 0.022 |
| Corticosteroids | GC-TOF | GLYCERIC_ACID | 0.022 |
| Anxioytic | LC-ECA | 13_78_992 | 0.023 |
| Antipsychotic | LC-ECA | 4HPLA | 0.023 |
| Antidepressant | GC-TOF | 219683 | 0.024 |
| Corticosteroids | GC-TOF | 231544 | 0.024 |
| Anxioytic | GC-TOF | 300379 | 0.024 |
| Corticosteroids | GC-TOF | ERYTHRITOL | 0.024 |
| Anxioytic | GC-TOF | 218597 | 0.025 |
| Antipsychotic | GC-TOF | 293097 | 0.025 |
| Antipsychotic | LC-ECA | 13_78_992 | 0.025 |
| Antipsychotic | LC-ECA | 2HPAC | 0.025 |
| Antipsychotic | LC-ECA | 14_22_758 | 0.026 |
| Anxioytic | GC-TOF | FRUCTOSE | 0.027 |
| Corticosteroids | LC-ECA | 13_19_492 | 0.029 |
| Statins | GC-TOF | 224849 | 0.03 |
| Anxioytic | GC-TOF | GLUTAMINE_DEH_{—} | 0.03 |
| Anxioytic | LC-ECA | I3AA | 0.03 |
| Anxioytic | LC-ECA | I3PA | 0.03 |
| Antipsychotic | LC-ECA | 13_18_475 | 0.03 |
| Anxioytic | GC-TOF | 228147 | 0.033 |
| Antipsychotic | GC-TOF | 231674 | 0.033 |
| Anxioytic | GC-TOF | 268321 | 0.033 |
| Anxioytic | GC-TOF | UREA | 0.033 |
| Corticosteroids | GC-TOF | GLUCOHEPTOSE | 0.034 |
| Statins | LC-ECA | GSH | 0.034 |
| Corticosteroids | GC-TOF | 200595 | 0.035 |
| Anxioytic | GC-TOF | 236890 | 0.035 |
| Statins | GC-TOF | 268420 | 0.035 |
| Antidepressant | GC-TOF | 301825 | 0.035 |
| Corticosteroids | GC-TOF | 308185 | 0.035 |
| Corticosteroids | LC-ECA | 11_36_75 | 0.035 |
| Anxioytic | GC-TOF | 202572 | 0.036 |
| Antidepressant | GC-TOF | 224035 | 0.036 |
| Anxioytic | GC-TOF | RIBOSE | 0.036 |
| Statins | LC-ECA | 8_28_508 | 0.036 |
| Anxioytic | GC-TOF | 3_HYDROXYBUTANOIC_ACID | 0.037 |
| Antidepressant | LC-ECA | 11_60_917 | 0.037 |
| Anxioytic | GC-TOF | BENZOIC_ACID | 0.038 |
| Corticosteroids | GC-TOF | PALMITIC_ACID | 0.038 |
| Anxioytic | LC-ECA | 9_19_067 | 0.038 |
| Antidepressant | GC-TOF | 213198 | 0.039 |
| Corticosteroids | GC-TOF | 3_HYDROXYBUTANOIC_ACID | 0.039 |
| Antipsychotic | LC-ECA | HVA | 0.039 |
| Statins | LC-ECA | 9_29_34 | 0.04 |
| Corticosteroids | GC-TOF | 234015 | 0.041 |
| Anxioytic | GC-TOF | 309532 | 0.042 |
| Anxioytic | GC-TOF | 312902 | 0.042 |
| Corticosteroids | LC-ECA | 9_29_925 | 0.042 |
| Corticosteroids | GC-TOF | 204425 | 0.043 |
| Antipsychotic | LC-ECA | 13_44_608 | 0.044 |
| Corticosteroids | GC-TOF | ERYTHROSE | 0.045 |
| Anxioytic | LC-ECA | 13_44_608 | 0.046 |
| Corticosteroids | GC-TOF | 218787 | 0.047 |
| Corticosteroids | GC-TOF | 212208 | 0.048 |
| Statins | GC-TOF | GLYCEROL | 0.048 |
| Anxioytic | GC-TOF | N_METHYLALANINE | 0.048 |
| Antipsychotic | LC-ECA | 13_74_392 | 0.048 |
| Antipsychotic | LC-ECA | 8_14_983 | 0.048 |
| Anxioytic | GC-TOF | 280546 | 0.049 |
| Antidepressant | GC-TOF | 231674 | 0.05 |
| Anxioytic | GC-TOF | 267884 | 0.05 |
| Antidepressant | GC-TOF | 267665 | 0.051 |
| Anxioytic | LC-ECA | TRP | 0.051 |
| Antipsychotic | LC-ECA | 14_64_275 | 0.051 |
| Anxioytic | LC-ECA | 15_90_6 | 0.051 |
| Antidepressant | GC-TOF | 200556 | 0.052 |
| Antidepressant | GC-TOF | 202572 | 0.052 |
| Statins | GC-TOF | N_ACETYL_D_MANNOSAMINE | 0.052 |
| Anxioytic | LC-ECA | 5_102_808 | 0.052 |
| Corticosteroids | GC-TOF | 310831 | 0.053 |
| Corticosteroids | GC-TOF | 200490 | 0.054 |
| Statins | GC-TOF | 213182 | 0.054 |
| Antidepressant | GC-TOF | 225863 | 0.054 |
| Antipsychotic | GC-TOF | 233005 | 0.054 |
| Statins | GC-TOF | 267884 | 0.054 |
| Anxioytic | GC-TOF | SERINE | 0.054 |
| Statins | LC-ECA | HVA | 0.054 |
| Corticosteroids | GC-TOF | 2_DEOXYRIBONIC_ACID | 0.055 |
| Corticosteroids | GC-TOF | 280940 | 0.055 |
| Anxioytic | GC-TOF | PELARGONIC_ACID | 0.055 |
| Corticosteroids | LC-ECA | 5_15_692 | 0.055 |
| Antidepressant | GC-TOF | 293849 | 0.056 |
| Anxioytic | GC-TOF | 301325 | 0.056 |
| Statins | LC-ECA | MHPG | 0.056 |
| Antidepressant | GC-TOF | 208755 | 0.057 |
| Antipsychotic | GC-TOF | N_METHYLALANINE | 0.058 |
| Statins | LC-ECA | 12_41_200 | 0.058 |
| Antidepressant | LC-ECA | 14_22_758 | 0.058 |
| Antipsychotic | LC-ECA | 8_93_65 | 0.058 |
| Statins | LC-ECA | XAN | 0.058 |
| Antidepressant | GC-TOF | 204318 | 0.059 |
| Anxioytic | GC-TOF | 226935 | 0.059 |
| Antipsychotic | LC-ECA | 11_46_55 | 0.059 |
| Anxioytic | GC-TOF | 213697 | 0.06 |
| Statins | GC-TOF | STEARIC_ACID | 0.06 |
| Anxioytic | GC-TOF | 228612 | 0.061 |
| Corticosteroids | GC-TOF | 3_DEOXYPENTITOL_NIST | 0.061 |
| Antidepressant | GC-TOF | 3_HYDROXYBUTANOIC_ACID | 0.061 |
| Anxioytic | GC-TOF | 221597 | 0.062 |
| Antidepressant | GC-TOF | ENOLPYRUVATE_NIST | 0.062 |
| Anxioytic | LC-ECA | 4_22_117 | 0.062 |
| Corticosteroids | GC-TOF | BETA_MANNOSYLGLYCERATE_MINOR | 0.063 |
| Anxioytic | LC-ECA | 12_41_200 | 0.063 |
| Antidepressant | LC-ECA | 13_44_608 | 0.063 |
| Antidepressant | GC-TOF | ALPHA_KETOGLUTARIC_ACID | 0.064 |
| Antipsychotic | GC-TOF | 218951 | 0.066 |
| Corticosteroids | GC-TOF | 223535 | 0.066 |
| Anxioytic | GC-TOF | THREONINE | 0.066 |
| Anxioytic | LC-ECA | 4HPLA | 0.066 |
| Corticosteroids | LC-ECA | 8_89_433 | 0.066 |
| Anxioytic | LC-ECA | 13_84_975 | 0.067 |
| Antipsychotic | GC-TOF | TAURINE | 0.068 |
| Anxioytic | LC-ECA | 14_64_275 | 0.069 |
| Antidepressant | GC-TOF | 225548 | 0.072 |
| Antidepressant | GC-TOF | 294129 | 0.072 |
| Anxioytic | GC-TOF | 306152 | 0.072 |
| Antidepressant | GC-TOF | 309873 | 0.072 |
| Anxioytic | GC-TOF | ERYTHROSE | 0.073 |
| Anxioytic | GC-TOF | PALMITIC_ACID | 0.073 |
| Anxioytic | LC-ECA | ASA | 0.073 |
| Corticosteroids | GC-TOF | 293097 | 0.074 |
| Antipsychotic | GC-TOF | CYSTEINE | 0.074 |
| Anxioytic | GC-TOF | 210168 | 0.075 |
| Antipsychotic | GC-TOF | 229164 | 0.075 |
| Corticosteroids | GC-TOF | CHOLESTEROL_{—} | 0.075 |
| Antipsychotic | GC-TOF | FRUCTOSE | 0.076 |
| Antipsychotic | LC-ECA | 15_68_542 | 0.076 |
| Anxioytic | GC-TOF | 228369 | 0.077 |
| Corticosteroids | GC-TOF | GLYCEROL_3_GALACTOSIDE | 0.077 |
| Statins | GC-TOF | XYLOSE | 0.077 |
| Statins | LC-ECA | 5HIAA | 0.077 |
| Antidepressant | GC-TOF | 204994 | 0.078 |
| Statins | LC-ECA | 4_32_592 | 0.078 |
| Antidepressant | GC-TOF | 306152 | 0.079 |
| Antidepressant | GC-TOF | 201005 | 0.08 |
| Antidepressant | GC-TOF | N_ACETYL_D_MANNOSAMINE | 0.08 |
| Antidepressant | LC-ECA | 2HPAC | 0.08 |
| Corticosteroids | GC-TOF | 238149 | 0.081 |
| Anxioytic | GC-TOF | GLYCERIC_ACID | 0.082 |
| Antidepressant | GC-TOF | 233005 | 0.083 |
| Antidepressant | GC-TOF | 268420 | 0.083 |
| Antipsychotic | GC-TOF | GLYCEROL_ALPHA_PHOSPHATE | 0.083 |
| Anxioytic | LC-ECA | 9_20_858 | 0.083 |
| Anxioytic | GC-TOF | 200556 | 0.084 |
| Corticosteroids | GC-TOF | 221597 | 0.084 |
| Anxioytic | LC-ECA | 5HTP | 0.085 |
| Anxioytic | GC-TOF | 2_DEOXYTETRONIC_ACID | 0.088 |
| Antidepressant | GC-TOF | 270351 | 0.088 |
| Antidepressant | GC-TOF | CYSTEINE | 0.088 |
| Statins | GC-TOF | PELARGONIC_ACID | 0.088 |
| Corticosteroids | GC-TOF | 289055 | 0.089 |
| Antidepressant | LC-ECA | URIC | 0.089 |
| Statins | GC-TOF | 208755 | 0.09 |
| Statins | GC-TOF | 224020 | 0.09 |
| Statins | GC-TOF | 268483 | 0.09 |
| Anxioytic | GC-TOF | 268365 | 0.091 |
| Statins | GC-TOF | 309538 | 0.091 |
| Antipsychotic | GC-TOF | 312592 | 0.091 |
| Antidepressant | LC-ECA | 12_41_200 | 0.092 |
| Corticosteroids | GC-TOF | FRUCTOSE | 0.093 |
| Anxioytic | GC-TOF | 219683 | 0.094 |
| Anxioytic | GC-TOF | 233005 | 0.094 |
| Corticosteroids | GC-TOF | 267884 | 0.094 |
| Corticosteroids | GC-TOF | 312308 | 0.094 |
| Antidepressant | LC-ECA | I3AA | 0.094 |
| Anxioytic | LC-ECA | 5_15_692 | 0.094 |
| Antidepressant | GC-TOF | 309934 | 0.095 |
| Antipsychotic | LC-ECA | MHPG | 0.095 |
| Antidepressant | LC-ECA | 5_24_483 | 0.095 |
| Antidepressant | GC-TOF | 1_MONOSTEARIN | 0.096 |
| Antidepressant | GC-TOF | 233340 | 0.096 |
| Corticosteroids | GC-TOF | FUCOSE_RHAMNOSE_{—} | 0.096 |
| Antidepressant | GC-TOF | 212208 | 0.097 |
| Anxioytic | GC-TOF | METHYLHEXADECANOIC_ACID | 0.097 |
| Anxioytic | GC-TOF | GLYCOLIC_ACID | 0.098 |
| Anxioytic | GC-TOF | 218767 | 0.099 |
| Antipsychotic | GC-TOF | 269160 | 0.099 |
| Antidepressant | GC-TOF | 312308 | 0.099 |
| Statins | GC-TOF | 203235 | 0.1 |
| Antipsychotic | GC-TOF | 217783 | 0.1 |
| Antidepressant | GC-TOF | 218597 | 0.1 |
| Antipsychotic | GC-TOF | 307889 | 0.1 |
| Antidepressant | GC-TOF | 309788 | 0.1 |
| Antipsychotic | GC-TOF | ETHANOLAMINE | 0.1 |
| Corticosteroids | GC-TOF | FUCOSE | 0.1 |
| Corticosteroids | GC-TOF | HEXURONIC_ACID | 0.1 |
| Anxioytic | GC-TOF | 199553 | 0.11 |
| Anxioytic | GC-TOF | 208686 | 0.11 |
| Corticosteroids | GC-TOF | 213182 | 0.11 |
| Corticosteroids | GC-TOF | 224635 | 0.11 |
| Anxioytic | GC-TOF | 226906 | 0.11 |
| Anxioytic | GC-TOF | 267649 | 0.11 |
| Anxioytic | GC-TOF | 310448 | 0.11 |
| Antidepressant | GC-TOF | CITRAMALATE | 0.11 |
| Anxioytic | GC-TOF | ERYTHRONIC_ACID_LACTONE | 0.11 |
| Statins | GC-TOF | GLYCERIC_ACID | 0.11 |
| Antidepressant | GC-TOF | MONOPALMITIN_1_GLYCERIDE | 0.11 |
| Antidepressant | GC-TOF | PHOSPHORIC_ACID | 0.11 |
| Antipsychotic | GC-TOF | RIBITOL | 0.11 |
| Antidepressant | GC-TOF | THREONIC_ACID | 0.11 |
| Anxioytic | GC-TOF | THYMINE | 0.11 |
| Anxioytic | LC-ECA | 5HIAA | 0.11 |
| Corticosteroids | GC-TOF | 213198 | 0.12 |
| Antipsychotic | GC-TOF | 224635 | 0.12 |
| Antidepressant | GC-TOF | 238149 | 0.12 |
| Antipsychotic | GC-TOF | 268313 | 0.12 |
| Statins | GC-TOF | 269160 | 0.12 |
| Corticosteroids | GC-TOF | 293848 | 0.12 |
| Corticosteroids | GC-TOF | 310006 | 0.12 |
| Anxioytic | GC-TOF | 312645 | 0.12 |
| Statins | GC-TOF | CYSTEINE | 0.12 |
| Antidepressant | GC-TOF | GLUCOSE | 0.12 |
| Corticosteroids | GC-TOF | METHYLHEXADECANOIC_ACID | 0.12 |
| Statins | GC-TOF | PALMITIC_ACID | 0.12 |
| Antipsychotic | GC-TOF | PHOSPHATE | 0.12 |
| Antipsychotic | LC-ECA | GR | 0.12 |
| Anxioytic | LC-ECA | MET | 0.12 |
| Statins | LC-ECA | PXAN | 0.12 |
| Corticosteroids | LC-ECA | 11_51_158 | 0.12 |
| Antipsychotic | LC-ECA | 8_28_508 | 0.12 |
| Statins | LC-ECA | 8_89_433 | 0.12 |
| Corticosteroids | GC-TOF | 199463 | 0.13 |
| Corticosteroids | GC-TOF | 2_MONOPALMITIN | 0.13 |
| Anxioytic | GC-TOF | 202573 | 0.13 |
| Anxioytic | GC-TOF | 204994 | 0.13 |
| Statins | GC-TOF | 212208 | 0.13 |
| Anxioytic | GC-TOF | 213143 | 0.13 |
| Antipsychotic | GC-TOF | 227652 | 0.13 |
| Antidepressant | GC-TOF | 229164 | 0.13 |
| Antidepressant | GC-TOF | 231099 | 0.13 |
| Corticosteroids | GC-TOF | 231674 | 0.13 |
| Anxioytic | GC-TOF | 239954 | 0.13 |
| Antipsychotic | GC-TOF | 268420 | 0.13 |
| Corticosteroids | GC-TOF | 296108 | 0.13 |
| Corticosteroids | GC-TOF | GLUCOHEPTULOSE | 0.13 |
| Corticosteroids | GC-TOF | MANNITOL | 0.13 |
| Anxioytic | GC-TOF | SUCCINIC_ACID | 0.13 |
| Statins | LC-ECA | 13_54_95 | 0.13 |
| Anxioytic | LC-ECA | 13_86_8 | 0.13 |
| Antidepressant | LC-ECA | 14_34_25 | 0.13 |
| Antipsychotic | LC-ECA | 8_89_433 | 0.13 |
| Antipsychotic | LC-ECA | 9_29_34 | 0.13 |
| Anxioytic | GC-TOF | 213227 | 0.14 |
| Corticosteroids | GC-TOF | 239954 | 0.14 |
| Anxioytic | GC-TOF | 241881 | 0.14 |
| Corticosteroids | GC-TOF | 242417 | 0.14 |
| Corticosteroids | GC-TOF | 273984 | 0.14 |
| Corticosteroids | GC-TOF | 280573 | 0.14 |
| Anxioytic | GC-TOF | 307889 | 0.14 |
| Anxioytic | GC-TOF | 312448 | 0.14 |
| Anxioytic | GC-TOF | PUTRESCINE | 0.14 |
| Corticosteroids | GC-TOF | STEARIC_ACID | 0.14 |
| Anxioytic | LC-ECA | GSH | 0.14 |
| Corticosteroids | LC-ECA | 13_74_392 | 0.14 |
| Antidepressant | LC-ECA | 4_32_592 | 0.14 |
| Antipsychotic | LC-ECA | 8_76_933 | 0.14 |
| Antidepressant | LC-ECA | XAN | 0.14 |
| Antipsychotic | GC-TOF | 201887 | 0.15 |
| Corticosteroids | GC-TOF | 208755 | 0.15 |
| Antidepressant | GC-TOF | 227597 | 0.15 |
| Antipsychotic | GC-TOF | 227597 | 0.15 |
| Antipsychotic | GC-TOF | 267650 | 0.15 |
| Anxioytic | GC-TOF | 267665 | 0.15 |
| Corticosteroids | GC-TOF | 281216 | 0.15 |
| Antidepressant | GC-TOF | 281907 | 0.15 |
| Anxioytic | GC-TOF | 309934 | 0.15 |
| Anxioytic | GC-TOF | 310006 | 0.15 |
| Statins | GC-TOF | DEHYDROASCORBATE | 0.15 |
| Corticosteroids | GC-TOF | LACTIC_ACID | 0.15 |
| Antidepressant | LC-ECA | ASA | 0.15 |
| Antidepressant | LC-ECA | 9_19_067 | 0.15 |
| Statins | GC-TOF | 2_DEOXYTETRONIC_ACID_NIST | 0.16 |
| Statins | GC-TOF | 2_HYDROXYVALERIC_ACID | 0.16 |
| Anxioytic | GC-TOF | 2_MONOSTEARIN_NIST | 0.16 |
| Anxioytic | GC-TOF | 208755 | 0.16 |
| Antidepressant | GC-TOF | 215978 | 0.16 |
| Statins | GC-TOF | 226935 | 0.16 |
| Corticosteroids | GC-TOF | 232604 | 0.16 |
| Corticosteroids | GC-TOF | 267649 | 0.16 |
| Corticosteroids | GC-TOF | 271416 | 0.16 |
| Statins | GC-TOF | 312289 | 0.16 |
| Anxioytic | GC-TOF | 312622 | 0.16 |
| Statins | GC-TOF | 312977 | 0.16 |
| Corticosteroids | GC-TOF | ARABITOL | 0.16 |
| Anxioytic | GC-TOF | CITRAMALATE | 0.16 |
| Anxioytic | GC-TOF | CONDURITOL_BETA_EPOXIDE | 0.16 |
| Corticosteroids | GC-TOF | MONOPALMITIN_1_GLYCERIDE | 0.16 |
| Anxioytic | GC-TOF | SORBITOL | 0.16 |
| Antipsychotic | GC-TOF | THYMINE | 0.16 |
| Antipsychotic | GC-TOF | VALINE | 0.16 |
| Anxioytic | GC-TOF | XYLOSE | 0.16 |
| Statins | LC-ECA | 11_60_917 | 0.16 |
| Corticosteroids | LC-ECA | 15_90_6 | 0.16 |
| Antidepressant | LC-ECA | 5_102_808 | 0.16 |
| Statins | LC-ECA | 5_102_808 | 0.16 |
| Antipsychotic | LC-ECA | XANTH | 0.16 |
| Statins | GC-TOF | 2_MONOPALMITIN | 0.17 |
| Corticosteroids | GC-TOF | 201887 | 0.17 |
| Corticosteroids | GC-TOF | 202091 | 0.17 |
| Anxioytic | GC-TOF | 213193 | 0.17 |
| Anxioytic | GC-TOF | 288808 | 0.17 |
| Antidepressant | GC-TOF | DEHYDROASCORBATE | 0.17 |
| Anxioytic | GC-TOF | ISOTHREONIC_ACID | 0.17 |
| Anxioytic | GC-TOF | LEUCINE | 0.17 |
| Corticosteroids | GC-TOF | PANTOTHENIC_ACID | 0.17 |
| Corticosteroids | LC-ECA | HX | 0.17 |
| Statins | LC-ECA | 15_77_017 | 0.17 |
| Corticosteroids | GC-TOF | 200541 | 0.18 |
| Anxioytic | GC-TOF | 218513 | 0.18 |
| Corticosteroids | GC-TOF | 227387 | 0.18 |
| Anxioytic | GC-TOF | 227582 | 0.18 |
| Statins | GC-TOF | 227582 | 0.18 |
| Anxioytic | GC-TOF | 227652 | 0.18 |
| Corticosteroids | GC-TOF | 227652 | 0.18 |
| Corticosteroids | GC-TOF | 234580 | 0.18 |
| Antipsychotic | GC-TOF | 238149 | 0.18 |
| Antipsychotic | GC-TOF | 242417 | 0.18 |
| Corticosteroids | GC-TOF | 267737 | 0.18 |
| Anxioytic | GC-TOF | 267816 | 0.18 |
| Anxioytic | GC-TOF | 289055 | 0.18 |
| Corticosteroids | GC-TOF | 303060 | 0.18 |
| Antidepressant | GC-TOF | ACETOPHENONE_NIST | 0.18 |
| Antipsychotic | GC-TOF | MANNITOL | 0.18 |
| Anxioytic | LC-ECA | URIC | 0.18 |
| Antidepressant | LC-ECA | 13_84_975 | 0.18 |
| Anxioytic | LC-ECA | 8_28_508 | 0.18 |
| Antipsychotic | LC-ECA | 8_82_917 | 0.18 |
| Antidepressant | LC-ECA | 9_29_925 | 0.18 |
| Antidepressant | GC-TOF | 2_DEOXYTETRONIC_ACID_NIST | 0.19 |
| Statins | GC-TOF | 204994 | 0.19 |
| Antipsychotic | GC-TOF | 213143 | 0.19 |
| Anxioytic | GC-TOF | 213198 | 0.19 |
| Corticosteroids | GC-TOF | 241920 | 0.19 |
| Antidepressant | GC-TOF | 280546 | 0.19 |
| Antipsychotic | GC-TOF | 309641 | 0.19 |
| Antidepressant | GC-TOF | 312362 | 0.19 |
| Antidepressant | GC-TOF | 312622 | 0.19 |
| Statins | GC-TOF | 4_HYDROXYBUTYRIC_ACID | 0.19 |
| Antipsychotic | GC-TOF | ALANINE | 0.19 |
| Anxioytic | GC-TOF | ETHANOLAMINE | 0.19 |
| Antipsychotic | GC-TOF | FUCOSE_RHAMNOSE_{—} | 0.19 |
| Antidepressant | GC-TOF | ISOCITRIC_ACID | 0.19 |
| Anxioytic | GC-TOF | MYRISTIC_ACID | 0.19 |
| Antidepressant | GC-TOF | PHOSPHATE | 0.19 |
| Antipsychotic | GC-TOF | XYLOSE | 0.19 |
| Anxioytic | LC-ECA | TYR | 0.19 |
| Statins | LC-ECA | 13_18_475 | 0.19 |
| Statins | LC-ECA | 13_78_992 | 0.19 |
| Statins | LC-ECA | 13_92_333 | 0.19 |
| Corticosteroids | LC-ECA | 5HIAA | 0.19 |
| Anxioytic | LC-ECA | 8_82_917 | 0.19 |
| Anxioytic | LC-ECA | 9_29_34 | 0.19 |
| Anxioytic | GC-TOF | 202885 | 0.2 |
| Antidepressant | GC-TOF | 226851 | 0.2 |
| Statins | GC-TOF | 231947 | 0.2 |
| Corticosteroids | GC-TOF | CONDURITOL_BETA_EPOXIDE | 0.2 |
| Statins | GC-TOF | ERYTHRONIC_ACID_LACTONE | 0.2 |
| Statins | GC-TOF | METHYLHEXADECANOIC_ACID | 0.2 |
| Antipsychotic | LC-ECA | 12_94_5 | 0.2 |
| Corticosteroids | LC-ECA | 12_94_5 | 0.2 |
| Corticosteroids | LC-ECA | 4_22_117 | 0.2 |
| Antipsychotic | GC-TOF | 210286 | 0.21 |
| Statins | GC-TOF | 215739 | 0.21 |
| Corticosteroids | GC-TOF | 225548 | 0.21 |
| Corticosteroids | GC-TOF | 226853 | 0.21 |
| Antipsychotic | GC-TOF | 228612 | 0.21 |
| Anxioytic | GC-TOF | 280940 | 0.21 |
| Corticosteroids | GC-TOF | ARABINOSE | 0.21 |
| Antipsychotic | GC-TOF | N_ACETYL_D_MANNOSAMINE | 0.21 |
| Antipsychotic | GC-TOF | SERINE | 0.21 |
| Corticosteroids | GC-TOF | THREITOL_{—} | 0.21 |
| Corticosteroids | GC-TOF | THREONINE | 0.21 |
| Corticosteroids | GC-TOF | XANTHINE | 0.21 |
| Statins | LC-ECA | 4HPLA | 0.21 |
| Statins | GC-TOF | 226906 | 0.22 |
| Statins | GC-TOF | 228528 | 0.22 |
| Corticosteroids | GC-TOF | 228885 | 0.22 |
| Statins | GC-TOF | 231850 | 0.22 |
| Anxioytic | GC-TOF | 232604 | 0.22 |
| Statins | GC-TOF | 241097 | 0.22 |
| Corticosteroids | GC-TOF | 267665 | 0.22 |
| Antidepressant | GC-TOF | 267675 | 0.22 |
| Anxioytic | GC-TOF | 3_HYDROXY_3_METHYLGLUTARIC_ACID | 0.22 |
| Statins | GC-TOF | 301325 | 0.22 |
| Statins | GC-TOF | 307889 | 0.22 |
| Antipsychotic | GC-TOF | 312289 | 0.22 |
| Antipsychotic | GC-TOF | FUCOSE | 0.22 |
| Corticosteroids | GC-TOF | GLYCEROL | 0.22 |
| Antipsychotic | GC-TOF | ISOLEUCINE | 0.22 |
| Antipsychotic | GC-TOF | LEUCINE | 0.22 |
| Corticosteroids | GC-TOF | SUCCINIC_ACID | 0.22 |
| Antipsychotic | LC-ECA | TYR | 0.22 |
| Antipsychotic | LC-ECA | 11_36_75 | 0.22 |
| Anxioytic | LC-ECA | 15_68_542 | 0.22 |
| Anxioytic | LC-ECA | 2HPAC | 0.22 |
| Statins | GC-TOF | 204318 | 0.23 |
| Corticosteroids | GC-TOF | 210286 | 0.23 |
| Antipsychotic | GC-TOF | 212208 | 0.23 |
| Anxioytic | GC-TOF | 218787 | 0.23 |
| Antidepressant | GC-TOF | 223535 | 0.23 |
| Corticosteroids | GC-TOF | 224849 | 0.23 |
| Corticosteroids | GC-TOF | 227270 | 0.23 |
| Corticosteroids | GC-TOF | 236890 | 0.23 |
| Anxioytic | GC-TOF | 238270 | 0.23 |
| Antipsychotic | GC-TOF | 267737 | 0.23 |
| Anxioytic | GC-TOF | 268709 | 0.23 |
| Antidepressant | GC-TOF | 269160 | 0.23 |
| Antidepressant | GC-TOF | 281257 | 0.23 |
| Corticosteroids | GC-TOF | 308328 | 0.23 |
| Anxioytic | GC-TOF | 312977 | 0.23 |
| Antidepressant | GC-TOF | ALANINE | 0.23 |
| Anxioytic | GC-TOF | GLUTAMIC_ACID | 0.23 |
| Anxioytic | GC-TOF | GLYCEROL | 0.23 |
| Anxioytic | GC-TOF | ISOCITRIC_ACID | 0.23 |
| Statins | LC-ECA | 11_36_75 | 0.23 |
| Antipsychotic | LC-ECA | 12_50_183 | 0.23 |
| Antidepressant | LC-ECA | 14_36_45 | 0.23 |
| Corticosteroids | LC-ECA | 14_64_275 | 0.23 |
| Antidepressant | LC-ECA | 5_25_075 | 0.23 |
| Antipsychotic | GC-TOF | 213193 | 0.24 |
| Corticosteroids | GC-TOF | 226906 | 0.24 |
| Antidepressant | GC-TOF | 231709 | 0.24 |
| Corticosteroids | GC-TOF | 270407 | 0.24 |
| Anxioytic | GC-TOF | 296108 | 0.24 |
| Antipsychotic | GC-TOF | 3_DEOXYPENTITOL_NIST | 0.24 |
| Antidepressant | GC-TOF | 306157 | 0.24 |
| Antidepressant | GC-TOF | ACONITIC_ACID | 0.24 |
| Antidepressant | GC-TOF | ASPARTIC_ACID | 0.24 |
| Antipsychotic | GC-TOF | BETA_MANNOSYLGLYCERATE_MINOR_{—} | 0.24 |
| Antidepressant | GC-TOF | CITRIC_ACID | 0.24 |
| Antidepressant | GC-TOF | ERYTHRONIC_ACID_LACTONE | 0.24 |
| Antipsychotic | GC-TOF | PHOSPHORIC_ACID | 0.24 |
| Anxioytic | LC-ECA | 13_19_492 | 0.24 |
| Anxioytic | GC-TOF | 199203 | 0.25 |
| Antidepressant | GC-TOF | 2_DEOXYRIBONIC_ACID | 0.25 |
| Anxioytic | GC-TOF | 201005 | 0.25 |
| Anxioytic | GC-TOF | 212208 | 0.25 |
| Anxioytic | GC-TOF | 216428 | 0.25 |
| Antipsychotic | GC-TOF | 221597 | 0.25 |
| Antipsychotic | GC-TOF | 226906 | 0.25 |
| Antipsychotic | GC-TOF | 228605 | 0.25 |
| Statins | GC-TOF | 241881 | 0.25 |
| Antidepressant | GC-TOF | 241920 | 0.25 |
| Statins | GC-TOF | 242417 | 0.25 |
| Corticosteroids | GC-TOF | 268579 | 0.25 |
| Statins | GC-TOF | 3_HYDROXY_3_METHYLGLUTARIC_ACID | 0.25 |
| Statins | GC-TOF | 312622 | 0.25 |
| Antipsychotic | GC-TOF | DEHYDROASCORBATE | 0.25 |
| Antipsychotic | GC-TOF | ISOTHREONIC_ACID | 0.25 |
| Statins | GC-TOF | THREONIC_ACID | 0.25 |
| Antidepressant | LC-ECA | 5HIAA | 0.25 |
| Statins | LC-ECA | 9_29_925 | 0.25 |
| Corticosteroids | GC-TOF | 2_MONOSTEARIN_NIST | 0.26 |
| Corticosteroids | GC-TOF | 202572 | 0.26 |
| Statins | GC-TOF | 222169 | 0.26 |
| Antidepressant | GC-TOF | 228885 | 0.26 |
| Corticosteroids | GC-TOF | 231709 | 0.26 |
| Corticosteroids | GC-TOF | 231850 | 0.26 |
| Antidepressant | GC-TOF | 234580 | 0.26 |
| Antidepressant | GC-TOF | 288808 | 0.26 |
| Antipsychotic | GC-TOF | 293848 | 0.26 |
| Corticosteroids | GC-TOF | 293849 | 0.26 |
| Anxioytic | GC-TOF | 310831 | 0.26 |
| Corticosteroids | LC-ECA | I3PA | 0.26 |
| Statins | LC-ECA | 13_44_608 | 0.26 |
| Statins | LC-ECA | 14_36_45 | 0.26 |
| Antipsychotic | LC-ECA | 9_25_825 | 0.26 |
| Antipsychotic | GC-TOF | 2_HYDROXYBUTANOIC_ACID | 0.27 |
| Antipsychotic | GC-TOF | 217870 | 0.27 |
| Antipsychotic | GC-TOF | 226853 | 0.27 |
| Anxioytic | GC-TOF | 228885 | 0.27 |
| Antidepressant | GC-TOF | 231544 | 0.27 |
| Antipsychotic | GC-TOF | 238467 | 0.27 |
| Anxioytic | GC-TOF | 3_DEOXYPENTITOL_NIST | 0.27 |
| Anxioytic | GC-TOF | 308328 | 0.27 |
| Anxioytic | GC-TOF | VALINE | 0.27 |
| Antipsychotic | GC-TOF | XANTHINE | 0.27 |
| Antidepressant | LC-ECA | 15_77_017 | 0.27 |
| Statins | LC-ECA | 9_20_858 | 0.27 |
| Corticosteroids | GC-TOF | 2_HYDROXYBUTANOIC_ACID | 0.28 |
| Antipsychotic | GC-TOF | 200490 | 0.28 |
| Corticosteroids | GC-TOF | 218710 | 0.28 |
| Antipsychotic | GC-TOF | 223505 | 0.28 |
| Corticosteroids | GC-TOF | 226303 | 0.28 |
| Anxioytic | GC-TOF | 227387 | 0.28 |
| Statins | GC-TOF | 271416 | 0.28 |
| Anxioytic | GC-TOF | 273984 | 0.28 |
| Antidepressant | GC-TOF | 299416 | 0.28 |
| Antipsychotic | GC-TOF | 306157 | 0.28 |
| Antipsychotic | GC-TOF | 308328 | 0.28 |
| Corticosteroids | GC-TOF | GLUTAMINE_DEH_{—} | 0.28 |
| Antidepressant | GC-TOF | LACTIC_ACID | 0.28 |
| Anxioytic | LC-ECA | 13_54_95 | 0.28 |
| Corticosteroids | LC-ECA | 15_65_533 | 0.28 |
| Anxioytic | LC-ECA | 5_24_483 | 0.28 |
| Corticosteroids | GC-TOF | 199203 | 0.29 |
| Corticosteroids | GC-TOF | 2_DEOXYERYTHRITOL | 0.29 |
| Anxioytic | GC-TOF | 203765 | 0.29 |
| Antidepressant | GC-TOF | 204344 | 0.29 |
| Antipsychotic | GC-TOF | 226851 | 0.29 |
| Antidepressant | GC-TOF | 236890 | 0.29 |
| Corticosteroids | GC-TOF | 240432 | 0.29 |
| Antipsychotic | GC-TOF | GLYCINE | 0.29 |
| Antipsychotic | GC-TOF | PANTOTHENIC_ACID | 0.29 |
| Corticosteroids | GC-TOF | UREA | 0.29 |
| Corticosteroids | GC-TOF | XYLITOL | 0.29 |
| Statins | LC-ECA | 14_75_608 | 0.29 |
| Antipsychotic | GC-TOF | 202091 | 0.3 |
| Antidepressant | GC-TOF | 204425 | 0.3 |
| Antipsychotic | GC-TOF | 204425 | 0.3 |
| Antidepressant | GC-TOF | 213182 | 0.3 |
| Statins | GC-TOF | 219683 | 0.3 |
| Anxioytic | GC-TOF | 226851 | 0.3 |
| Anxioytic | GC-TOF | 227597 | 0.3 |
| Corticosteroids | GC-TOF | 228369 | 0.3 |
| Anxioytic | GC-TOF | 231099 | 0.3 |
| Antidepressant | GC-TOF | 231947 | 0.3 |
| Antipsychotic | GC-TOF | 239954 | 0.3 |
| Statins | GC-TOF | 240432 | 0.3 |
| Statins | GC-TOF | 241920 | 0.3 |
| Statins | GC-TOF | 269625 | 0.3 |
| Antidepressant | GC-TOF | 301583 | 0.3 |
| Antidepressant | GC-TOF | 312645 | 0.3 |
| Statins | GC-TOF | ASPARAGINE_DEH_{—} | 0.3 |
| Anxioytic | GC-TOF | ISOLEUCINE | 0.3 |
| Corticosteroids | GC-TOF | N_ACETYL_D_MANNOSAMINE | 0.3 |
| Antidepressant | GC-TOF | RIBOSE | 0.3 |
| Antidepressant | GC-TOF | UREA | 0.3 |
| Statins | LC-ECA | I3PA | 0.3 |
| Antipsychotic | GC-TOF | 2_DEOXYTETRONIC_ACID | 0.31 |
| Antipsychotic | GC-TOF | 2_DEOXYTETRONIC_ACID_NIST | 0.31 |
| Anxioytic | GC-TOF | 2_KETOISOCAPROIC_ACID | 0.31 |
| Antidepressant | GC-TOF | 200490 | 0.31 |
| Corticosteroids | GC-TOF | 223505 | 0.31 |
| Corticosteroids | GC-TOF | 281257 | 0.31 |
| Antipsychotic | GC-TOF | 301325 | 0.31 |
| Statins | GC-TOF | 306152 | 0.31 |
| Anxioytic | GC-TOF | ASPARTIC_ACID | 0.31 |
| Corticosteroids | GC-TOF | ERYTHRONIC_ACID_LACTONE | 0.31 |
| Statins | GC-TOF | IDONIC_ACID_NIST | 0.31 |
| Corticosteroids | GC-TOF | SALICYLALDEHYDE | 0.31 |
| Antidepressant | LC-ECA | VMA | 0.31 |
| Antipsychotic | LC-ECA | VMA | 0.31 |
| Statins | LC-ECA | 4HPAC | 0.31 |
| Antipsychotic | GC-TOF | 2_DEOXYRIBONIC_ACID | 0.32 |
| Corticosteroids | GC-TOF | 208655 | 0.32 |
| Antipsychotic | GC-TOF | 215978 | 0.32 |
| Corticosteroids | GC-TOF | 222169 | 0.32 |
| Antidepressant | GC-TOF | 228605 | 0.32 |
| Antidepressant | GC-TOF | 231326 | 0.32 |
| Antipsychotic | GC-TOF | 231544 | 0.32 |
| Antipsychotic | GC-TOF | 234622 | 0.32 |
| Corticosteroids | GC-TOF | 238467 | 0.32 |
| Corticosteroids | GC-TOF | 3_HYDROXY_3_METHYLGLUTARIC_ACID | 0.32 |
| Antidepressant | GC-TOF | DIHYDROXYMALONIC_ACID_NIST | 0.32 |
| Statins | GC-TOF | PANTOTHENIC_ACID | 0.32 |
| Corticosteroids | GC-TOF | RIBOSE | 0.32 |
| Antipsychotic | GC-TOF | TRYPTOPHAN | 0.32 |
| Statins | LC-ECA | 12_52_75 | 0.32 |
| Corticosteroids | LC-ECA | 13_54_95 | 0.32 |
| Anxioytic | LC-ECA | 15_65_533 | 0.32 |
| Anxioytic | GC-TOF | 217783 | 0.33 |
| Antidepressant | GC-TOF | 228612 | 0.33 |
| Corticosteroids | GC-TOF | 231947 | 0.33 |
| Corticosteroids | GC-TOF | 312362 | 0.33 |
| Antidepressant | GC-TOF | 312679 | 0.33 |
| Statins | GC-TOF | ERYTHROSE | 0.33 |
| Antidepressant | GC-TOF | INOSITOL_ALLO_{—} | 0.33 |
| Anxioytic | GC-TOF | INOSITOL_MYO_{—} | 0.33 |
| Corticosteroids | GC-TOF | INOSITOL_MYO_{—} | 0.33 |
| Corticosteroids | GC-TOF | LYSINE | 0.33 |
| Antidepressant | GC-TOF | SERINE | 0.33 |
| Antipsychotic | GC-TOF | SUCROSE | 0.33 |
| Antipsychotic | LC-ECA | MET | 0.33 |
| Statins | LC-ECA | 13_38_49 | 0.33 |
| Antidepressant | LC-ECA | 8_14_983 | 0.33 |
| Statins | LC-ECA | 8_82_917 | 0.33 |
| Antipsychotic | GC-TOF | 226935 | 0.34 |
| Antidepressant | GC-TOF | 228147 | 0.34 |
| Antidepressant | GC-TOF | 242417 | 0.34 |
| Statins | GC-TOF | 267816 | 0.34 |
| Antidepressant | GC-TOF | 280940 | 0.34 |
| Antidepressant | GC-TOF | 309642 | 0.34 |
| Statins | GC-TOF | 309934 | 0.34 |
| Antipsychotic | GC-TOF | ASPARTIC_ACID | 0.34 |
| Antidepressant | GC-TOF | ERYTHRITOL | 0.34 |
| Antidepressant | GC-TOF | INOSITOL_MYO_{—} | 0.34 |
| Antipsychotic | GC-TOF | PUTREANINE_NIST | 0.34 |
| Statins | GC-TOF | SALICYLALDEHYDE | 0.34 |
| Antidepressant | LC-ECA | GSH | 0.34 |
| Statins | LC-ECA | HX | 0.34 |
| Antipsychotic | LC-ECA | 12_52_75 | 0.34 |
| Statins | LC-ECA | 13_74_392 | 0.34 |
| Corticosteroids | GC-TOF | 1_MONOSTEARIN | 0.35 |
| Corticosteroids | GC-TOF | 2_DEOXYTETRONIC_ACID_NIST | 0.35 |
| Statins | GC-TOF | 2_MONOSTEARIN_NIST | 0.35 |
| Statins | GC-TOF | 204344 | 0.35 |
| Corticosteroids | GC-TOF | 212274 | 0.35 |
| Antipsychotic | GC-TOF | 222169 | 0.35 |
| Anxioytic | GC-TOF | 224849 | 0.35 |
| Corticosteroids | GC-TOF | 228557 | 0.35 |
| Antipsychotic | GC-TOF | 232485 | 0.35 |
| Antipsychotic | GC-TOF | 273984 | 0.35 |
| Antipsychotic | GC-TOF | 308185 | 0.35 |
| Anxioytic | GC-TOF | N_ACETYL_D_MANNOSAMINE | 0.35 |
| Antidepressant | GC-TOF | SALICYLALDEHYDE | 0.35 |
| Corticosteroids | GC-TOF | XYLOSE | 0.35 |
| Corticosteroids | GC-TOF | XYLULOSE_NIST | 0.35 |
| Antidepressant | LC-ECA | 13_54_95 | 0.35 |
| Antidepressant | LC-ECA | 8_76_933 | 0.35 |
| Antipsychotic | GC-TOF | 200595 | 0.36 |
| Antipsychotic | GC-TOF | 202885 | 0.36 |
| Statins | GC-TOF | 210286 | 0.36 |
| Antidepressant | GC-TOF | 217783 | 0.36 |
| Anxioytic | GC-TOF | 218710 | 0.36 |
| Anxioytic | GC-TOF | 225863 | 0.36 |
| Antidepressant | GC-TOF | 270407 | 0.36 |
| Statins | GC-TOF | 273984 | 0.36 |
| Antidepressant | GC-TOF | 289055 | 0.36 |
| Statins | GC-TOF | 312645 | 0.36 |
| Corticosteroids | GC-TOF | 4_HYDROXYBUTYRIC_ACID | 0.36 |
| Anxioytic | GC-TOF | GLUCONIC_ACID | 0.36 |
| Antidepressant | GC-TOF | METHIONINE | 0.36 |
| Corticosteroids | GC-TOF | OXOPROLINE | 0.36 |
| Anxioytic | GC-TOF | THREITOL_{—} | 0.36 |
| Antipsychotic | GC-TOF | TYROSINE | 0.36 |
| Antidepressant | LC-ECA | 13_86_8 | 0.36 |
| Anxioytic | LC-ECA | 4HPAC | 0.36 |
| Anxioytic | LC-ECA | XANTH | 0.36 |
| Corticosteroids | GC-TOF | 231657 | 0.37 |
| Antipsychotic | GC-TOF | 232604 | 0.37 |
| Statins | GC-TOF | 281907 | 0.37 |
| Antipsychotic | GC-TOF | 301536 | 0.37 |
| Statins | GC-TOF | 312902 | 0.37 |
| Antipsychotic | GC-TOF | INOSITOL_MYO_{—} | 0.37 |
| Antidepressant | GC-TOF | XYLITOL | 0.37 |
| Corticosteroids | LC-ECA | HVA | 0.37 |
| Antipsychotic | LC-ECA | 13_54_95 | 0.37 |
| Antipsychotic | LC-ECA | 15_65_533 | 0.37 |
| Statins | LC-ECA | 5_25_075 | 0.37 |
| Antidepressant | LC-ECA | XANTH | 0.37 |
| Statins | GC-TOF | 268313 | 0.38 |
| Antidepressant | GC-TOF | 268483 | 0.38 |
| Anxioytic | GC-TOF | 270351 | 0.38 |
| Antidepressant | GC-TOF | 312592 | 0.38 |
| Corticosteroids | GC-TOF | PHENYLALANINE | 0.38 |
| Corticosteroids | GC-TOF | PHOSPHORIC_ACID | 0.38 |
| Antipsychotic | LC-ECA | 13_84_975 | 0.38 |
| Antidepressant | LC-ECA | 15_65_533 | 0.38 |
| Corticosteroids | LC-ECA | 5_25_075 | 0.38 |
| Corticosteroids | LC-ECA | 9_20_858 | 0.38 |
| Corticosteroids | GC-TOF | 204344 | 0.39 |
| Antipsychotic | GC-TOF | 210168 | 0.39 |
| Antidepressant | GC-TOF | 212274 | 0.39 |
| Statins | GC-TOF | 213193 | 0.39 |
| Statins | GC-TOF | 227655 | 0.39 |
| Anxioytic | GC-TOF | 288019 | 0.39 |
| Corticosteroids | GC-TOF | 288808 | 0.39 |
| Antidepressant | GC-TOF | 310006 | 0.39 |
| Statins | GC-TOF | 312362 | 0.39 |
| Corticosteroids | GC-TOF | BENZOIC_ACID | 0.39 |
| Anxioytic | GC-TOF | GLUCOHEPTOSE | 0.39 |
| Anxioytic | GC-TOF | GLUCOSE | 0.39 |
| Antipsychotic | GC-TOF | THREITOL_{—} | 0.39 |
| Antidepressant | LC-ECA | HVA | 0.39 |
| Anxioytic | GC-TOF | 200595 | 0.4 |
| Antipsychotic | GC-TOF | 223535 | 0.4 |
| Statins | GC-TOF | 223535 | 0.4 |
| Statins | GC-TOF | 224322 | 0.4 |
| Statins | GC-TOF | 226851 | 0.4 |
| Antidepressant | GC-TOF | 227270 | 0.4 |
| Statins | GC-TOF | 228369 | 0.4 |
| Antidepressant | GC-TOF | 228911 | 0.4 |
| Anxioytic | GC-TOF | 271416 | 0.4 |
| Statins | GC-TOF | 296106 | 0.4 |
| Corticosteroids | GC-TOF | 309545 | 0.4 |
| Statins | GC-TOF | GLUCOHEPTULOSE | 0.4 |
| Corticosteroids | GC-TOF | PSEUDO_URIDINE | 0.4 |
| Antipsychotic | LC-ECA | TRP | 0.4 |
| Antipsychotic | LC-ECA | 13_92_333 | 0.4 |
| Antipsychotic | LC-ECA | 5_15_692 | 0.4 |
| Corticosteroids | LC-ECA | 9_29_34 | 0.4 |
| Corticosteroids | GC-TOF | 199806 | 0.41 |
| Antidepressant | GC-TOF | 2_DEOXYTETRONIC_ACID | 0.41 |
| Antipsychotic | GC-TOF | 2_KETOISOCAPROIC_ACID | 0.41 |
| Antidepressant | GC-TOF | 215494 | 0.41 |
| Antipsychotic | GC-TOF | 224035 | 0.41 |
| Statins | GC-TOF | 228911 | 0.41 |
| Statins | GC-TOF | 231657 | 0.41 |
| Anxioytic | GC-TOF | 231709 | 0.41 |
| Statins | GC-TOF | 234015 | 0.41 |
| Antidepressant | GC-TOF | 235414 | 0.41 |
| Corticosteroids | GC-TOF | 288019 | 0.41 |
| Corticosteroids | GC-TOF | 299416 | 0.41 |
| Antidepressant | GC-TOF | 300379 | 0.41 |
| Corticosteroids | GC-TOF | 301825 | 0.41 |
| Antidepressant | GC-TOF | 307965 | 0.41 |
| Corticosteroids | GC-TOF | CREATININE | 0.41 |
| Anxioytic | GC-TOF | GLYCEROL_3_GALACTOSIDE | 0.41 |
| Corticosteroids | GC-TOF | PHOSPHOETHANOLAMINE | 0.41 |
| Corticosteroids | GC-TOF | VALINE | 0.41 |
| Anxioytic | GC-TOF | 199806 | 0.42 |
| Antidepressant | GC-TOF | 203765 | 0.42 |
| Antipsychotic | GC-TOF | 211935 | 0.42 |
| Corticosteroids | GC-TOF | 218767 | 0.42 |
| Antidepressant | GC-TOF | 223505 | 0.42 |
| Antipsychotic | GC-TOF | 240432 | 0.42 |
| Corticosteroids | GC-TOF | 268420 | 0.42 |
| Anxioytic | GC-TOF | 270407 | 0.42 |
| Antipsychotic | GC-TOF | 271416 | 0.42 |
| Corticosteroids | GC-TOF | ETHANOLAMINE | 0.42 |
| Corticosteroids | GC-TOF | N_METHYLALANINE | 0.42 |
| Antipsychotic | LC-ECA | HX | 0.42 |
| Anxioytic | LC-ECA | 13_92_333 | 0.42 |
| Antidepressant | LC-ECA | 4HBAC | 0.42 |
| Antipsychotic | LC-ECA | 8_63_675 | 0.42 |
| Antidepressant | GC-TOF | 2_HYDROXYVALERIC_ACID | 0.43 |
| Antidepressant | GC-TOF | 221597 | 0.43 |
| Statins | GC-TOF | 224035 | 0.43 |
| Corticosteroids | GC-TOF | 235414 | 0.43 |
| Corticosteroids | GC-TOF | 296106 | 0.43 |
| Antidepressant | GC-TOF | 309641 | 0.43 |
| Anxioytic | GC-TOF | HEXURONIC_ACID | 0.43 |
| Antipsychotic | GC-TOF | MYRISTIC_ACID | 0.43 |
| Corticosteroids | LC-ECA | MET | 0.43 |
| Anxioytic | LC-ECA | 11_51_158 | 0.43 |
| Statins | LC-ECA | 14_64_275 | 0.43 |
| Antidepressant | GC-TOF | 2_KETOISOCAPROIC_ACID | 0.44 |
| Statins | GC-TOF | 206308 | 0.44 |
| Statins | GC-TOF | 213143 | 0.44 |
| Antidepressant | GC-TOF | 213193 | 0.44 |
| Anxioytic | GC-TOF | 226853 | 0.44 |
| Anxioytic | GC-TOF | 228557 | 0.44 |
| Statins | GC-TOF | 238467 | 0.44 |
| Antipsychotic | GC-TOF | 241097 | 0.44 |
| Antipsychotic | GC-TOF | 241881 | 0.44 |
| Antipsychotic | GC-TOF | 268483 | 0.44 |
| Corticosteroids | GC-TOF | 269625 | 0.44 |
| Antidepressant | GC-TOF | 288019 | 0.44 |
| Statins | GC-TOF | 309873 | 0.44 |
| Corticosteroids | GC-TOF | ALPHA_KETOGLUTARIC_ACID | 0.44 |
| Anxioytic | GC-TOF | ARABITOL | 0.44 |
| Antidepressant | GC-TOF | ASPARAGINE_DEH_{—} | 0.44 |
| Corticosteroids | GC-TOF | DIHYDROXYMALONIC_ACID_NIST | 0.44 |
| Antipsychotic | GC-TOF | GLYCEROL_3_GALACTOSIDE | 0.44 |
| Anxioytic | GC-TOF | MALTOSE_1 | 0.44 |
| Antidepressant | GC-TOF | PALMITIC_ACID | 0.44 |
| Antipsychotic | GC-TOF | PHENYLALANINE | 0.44 |
| Antipsychotic | GC-TOF | UREA | 0.44 |
| Antipsychotic | GC-TOF | XYLITOL | 0.44 |
| Corticosteroids | LC-ECA | TYR | 0.44 |
| Antipsychotic | LC-ECA | URIC | 0.44 |
| Statins | LC-ECA | 11_46_55 | 0.44 |
| Anxioytic | LC-ECA | 13_18_475 | 0.44 |
| Antidepressant | LC-ECA | 13_78_992 | 0.44 |
| Anxioytic | GC-TOF | 2_MONOPALMITIN | 0.45 |
| Corticosteroids | GC-TOF | 216428 | 0.45 |
| Corticosteroids | GC-TOF | 218513 | 0.45 |
| Antipsychotic | GC-TOF | 218767 | 0.45 |
| Statins | GC-TOF | 225548 | 0.45 |
| Corticosteroids | GC-TOF | 238270 | 0.45 |
| Corticosteroids | GC-TOF | 267675 | 0.45 |
| Antipsychotic | GC-TOF | 281216 | 0.45 |
| Anxioytic | GC-TOF | 296106 | 0.45 |
| Antipsychotic | GC-TOF | 312645 | 0.45 |
| Antipsychotic | GC-TOF | GLUCOHEPTULOSE | 0.45 |
| Statins | GC-TOF | SUCCINIC_ACID | 0.45 |
| Statins | GC-TOF | XYLITOL | 0.45 |
| Corticosteroids | LC-ECA | GSH | 0.45 |
| Antipsychotic | LC-ECA | I3PA | 0.45 |
| Antidepressant | LC-ECA | 11_51_158 | 0.45 |
| Corticosteroids | LC-ECA | 15_68_542 | 0.45 |
| Antipsychotic | LC-ECA | 9_19_067 | 0.45 |
| Antidepressant | GC-TOF | 2_HYDROXYBUTANOIC_ACID | 0.46 |
| Corticosteroids | GC-TOF | 203765 | 0.46 |
| Statins | GC-TOF | 231097 | 0.46 |
| Antidepressant | GC-TOF | 268579 | 0.46 |
| Antipsychotic | GC-TOF | 269625 | 0.46 |
| Statins | GC-TOF | 288019 | 0.46 |
| Antidepressant | GC-TOF | 301325 | 0.46 |
| Anxioytic | GC-TOF | 303060 | 0.46 |
| Antipsychotic | GC-TOF | 306156 | 0.46 |
| Antidepressant | GC-TOF | 309538 | 0.46 |
| Antipsychotic | GC-TOF | 310006 | 0.46 |
| Corticosteroids | GC-TOF | ASCORBIC_ACID | 0.46 |
| Antidepressant | GC-TOF | PUTRESCINE | 0.46 |
| Antipsychotic | GC-TOF | PUTRESCINE | 0.46 |
| Antidepressant | LC-ECA | 12_94_5 | 0.46 |
| Anxioytic | LC-ECA | 8_93_65 | 0.46 |
| Antipsychotic | GC-TOF | 2_MONOPALMITIN | 0.47 |
| Anxioytic | GC-TOF | 200490 | 0.47 |
| Statins | GC-TOF | 201005 | 0.47 |
| Anxioytic | GC-TOF | 203235 | 0.47 |
| Antipsychotic | GC-TOF | 224020 | 0.47 |
| Statins | GC-TOF | 226303 | 0.47 |
| Antidepressant | GC-TOF | 226935 | 0.47 |
| Antipsychotic | GC-TOF | 227270 | 0.47 |
| Antipsychotic | GC-TOF | 227582 | 0.47 |
| Statins | GC-TOF | 227652 | 0.47 |
| Antipsychotic | GC-TOF | 228369 | 0.47 |
| Anxioytic | GC-TOF | 228528 | 0.47 |
| Statins | GC-TOF | 236890 | 0.47 |
| Anxioytic | GC-TOF | 269625 | 0.47 |
| Corticosteroids | GC-TOF | 312645 | 0.47 |
| Antidepressant | GC-TOF | 312902 | 0.47 |
| Corticosteroids | GC-TOF | GLYCOLIC_ACID | 0.47 |
| Corticosteroids | GC-TOF | INOSITOL_ALLO_{—} | 0.47 |
| Anxioytic | GC-TOF | MANNITOL | 0.47 |
| Corticosteroids | GC-TOF | THYMINE | 0.47 |
| Anxioytic | LC-ECA | MHPG | 0.47 |
| Antidepressant | LC-ECA | 12_50_183 | 0.47 |
| Antipsychotic | GC-TOF | 199203 | 0.48 |
| Statins | GC-TOF | 1_MONOSTEARIN | 0.48 |
| Corticosteroids | GC-TOF | 200556 | 0.48 |
| Anxioytic | GC-TOF | 214426 | 0.48 |
| Anxioytic | GC-TOF | 224035 | 0.48 |
| Corticosteroids | GC-TOF | 300379 | 0.48 |
| Antipsychotic | GC-TOF | 309642 | 0.48 |
| Antipsychotic | GC-TOF | 309788 | 0.48 |
| Statins | GC-TOF | 312679 | 0.48 |
| Antidepressant | GC-TOF | CHOLESTEROL_{—} | 0.48 |
| Statins | GC-TOF | ETHANOLAMINE | 0.48 |
| Antidepressant | GC-TOF | FRUCTOSE | 0.48 |
| Statins | GC-TOF | FUCOSE_RHAMNOSE_{—} | 0.48 |
| Antipsychotic | GC-TOF | GLUCOHEPTOSE | 0.48 |
| Statins | GC-TOF | ISOTHREONIC_ACID | 0.48 |
| Antipsychotic | GC-TOF | METHIONINE | 0.48 |
| Antidepressant | GC-TOF | PHOSPHOETHANOLAMINE | 0.48 |
| Antidepressant | GC-TOF | SUCROSE | 0.48 |
| Corticosteroids | LC-ECA | VMA | 0.48 |
| Anxioytic | LC-ECA | 12_52_75 | 0.48 |
| Antipsychotic | LC-ECA | 14_75_608 | 0.48 |
| Corticosteroids | LC-ECA | 8_63_675 | 0.48 |
| Statins | LC-ECA | 9_19_067 | 0.48 |
| Anxioytic | GC-TOF | 200906 | 0.49 |
| Antidepressant | GC-TOF | 213227 | 0.49 |
| Antidepressant | GC-TOF | 213697 | 0.49 |
| Statins | GC-TOF | 218787 | 0.49 |
| Antidepressant | GC-TOF | 224322 | 0.49 |
| Corticosteroids | GC-TOF | 229164 | 0.49 |
| Antidepressant | GC-TOF | 273984 | 0.49 |
| Corticosteroids | GC-TOF | 294129 | 0.49 |
| Statins | GC-TOF | 3_HYDROXYBUTANOIC_ACID | 0.49 |
| Antipsychotic | GC-TOF | 307965 | 0.49 |
| Antipsychotic | GC-TOF | 310448 | 0.49 |
| Corticosteroids | GC-TOF | ASPARTIC_ACID | 0.49 |
| Antidepressant | GC-TOF | BENZOIC_ACID | 0.49 |
| Statins | GC-TOF | GLYCEROL_3_GALACTOSIDE | 0.49 |
| Antipsychotic | GC-TOF | LYSINE | 0.49 |
| Corticosteroids | GC-TOF | MYRISTIC_ACID | 0.49 |
| Antipsychotic | GC-TOF | SUCCINIC_ACID | 0.49 |
| Statins | GC-TOF | SUCROSE | 0.49 |
| Antidepressant | GC-TOF | TAURINE | 0.49 |
| Anxioytic | GC-TOF | XANTHINE | 0.49 |
| Corticosteroids | LC-ECA | ASA | 0.49 |
| Statins | LC-ECA | 5_40_292 | 0.49 |
| Anxioytic | LC-ECA | 8_14_983 | 0.49 |
| Statins | GC-TOF | 208655 | 0.5 |
| Antidepressant | GC-TOF | 210286 | 0.5 |
| Antidepressant | GC-TOF | 214426 | 0.5 |
| Anxioytic | GC-TOF | 215739 | 0.5 |
| Statins | GC-TOF | 216428 | 0.5 |
| Corticosteroids | GC-TOF | 223830 | 0.5 |
| Corticosteroids | GC-TOF | 224322 | 0.5 |
| Antidepressant | GC-TOF | 227387 | 0.5 |
| Corticosteroids | GC-TOF | 233005 | 0.5 |
| Antipsychotic | GC-TOF | 281257 | 0.5 |
| Corticosteroids | GC-TOF | 306159 | 0.5 |
| Statins | GC-TOF | 309545 | 0.5 |
| Statins | GC-TOF | BENZOIC_ACID | 0.5 |
| Corticosteroids | GC-TOF | CYSTEINE | 0.5 |
| Corticosteroids | GC-TOF | GLYCEROL_ALPHA_PHOSPHATE | 0.5 |
| Antipsychotic | GC-TOF | INULOBIOSE_2 | 0.5 |
| Statins | GC-TOF | MONOPALMITIN_1_GLYCERIDE | 0.5 |
| Statins | LC-ECA | TRP | 0.5 |
| Corticosteroids | GC-TOF | 2_DEOXYTETRONIC_ACID | 0.51 |
| Antipsychotic | GC-TOF | 204994 | 0.51 |
| Antidepressant | GC-TOF | 218787 | 0.51 |
| Statins | GC-TOF | 224551 | 0.51 |
| Statins | GC-TOF | 226853 | 0.51 |
| Antidepressant | GC-TOF | 228557 | 0.51 |
| Antipsychotic | GC-TOF | 231850 | 0.51 |
| Antidepressant | GC-TOF | 238467 | 0.51 |
| Antidepressant | GC-TOF | 268313 | 0.51 |
| Corticosteroids | GC-TOF | 269160 | 0.51 |
| Antipsychotic | GC-TOF | 309934 | 0.51 |
| Corticosteroids | GC-TOF | 312448 | 0.51 |
| Statins | GC-TOF | FUCOSE | 0.51 |
| Antipsychotic | GC-TOF | GLUTAMINE_DEH_{—} | 0.51 |
| Statins | GC-TOF | PROLINE | 0.51 |
| Antipsychotic | GC-TOF | RIBOSE | 0.51 |
| Antidepressant | GC-TOF | STEARIC_ACID | 0.51 |
| Statins | GC-TOF | UREA | 0.51 |
| Statins | LC-ECA | 15_65_533 | 0.51 |
| Antidepressant | GC-TOF | 199463 | 0.52 |
| Antipsychotic | GC-TOF | 199806 | 0.52 |
| Antidepressant | GC-TOF | 201887 | 0.52 |
| Anxioytic | GC-TOF | 212274 | 0.52 |
| Antidepressant | GC-TOF | 215739 | 0.52 |
| Corticosteroids | GC-TOF | 218951 | 0.52 |
| Antidepressant | GC-TOF | 231097 | 0.52 |
| Statins | GC-TOF | 238149 | 0.52 |
| Antidepressant | GC-TOF | 238270 | 0.52 |
| Antipsychotic | GC-TOF | 267649 | 0.52 |
| Anxioytic | GC-TOF | 267675 | 0.52 |
| Corticosteroids | GC-TOF | 309538 | 0.52 |
| Statins | GC-TOF | 310006 | 0.52 |
| Anxioytic | GC-TOF | BETA_MANNOSYLGLYCERATE_MINOR_{—} | 0.52 |
| Anxioytic | GC-TOF | CITRIC_ACID | 0.52 |
| Antidepressant | GC-TOF | GLYCEROL_3_GALACTOSIDE | 0.52 |
| Antidepressant | GC-TOF | PROLINE | 0.52 |
| Antidepressant | GC-TOF | XYLULOSE_NIST | 0.52 |
| Anxioytic | LC-ECA | 14_22_758 | 0.52 |
| Anxioytic | LC-ECA | 9_25_825 | 0.52 |
| Antidepressant | GC-TOF | 199777 | 0.53 |
| Statins | GC-TOF | 227270 | 0.53 |
| Statins | GC-TOF | 229164 | 0.53 |
| Antipsychotic | GC-TOF | 236890 | 0.53 |
| Corticosteroids | GC-TOF | 268483 | 0.53 |
| Corticosteroids | GC-TOF | 270351 | 0.53 |
| Antipsychotic | GC-TOF | 294129 | 0.53 |
| Corticosteroids | GC-TOF | 307965 | 0.53 |
| Statins | GC-TOF | 307965 | 0.53 |
| Corticosteroids | GC-TOF | 309642 | 0.53 |
| Antidepressant | GC-TOF | 312289 | 0.53 |
| Antipsychotic | GC-TOF | ACONITIC_ACID | 0.53 |
| Statins | GC-TOF | ARABINOSE | 0.53 |
| Corticosteroids | GC-TOF | CITRAMALATE | 0.53 |
| Antidepressant | GC-TOF | GLUCOHEPTOSE | 0.53 |
| Statins | GC-TOF | MYRISTIC_ACID | 0.53 |
| Antidepressant | LC-ECA | HX | 0.53 |
| Antipsychotic | LC-ECA | 5_25_075 | 0.53 |
| Corticosteroids | LC-ECA | XANTH | 0.53 |
| Corticosteroids | GC-TOF | 202599 | 0.54 |
| Corticosteroids | GC-TOF | 211935 | 0.54 |
| Antipsychotic | GC-TOF | 213198 | 0.54 |
| Antidepressant | GC-TOF | 218710 | 0.54 |
| Antidepressant | GC-TOF | 227652 | 0.54 |
| Corticosteroids | GC-TOF | 231097 | 0.54 |
| Statins | GC-TOF | 267649 | 0.54 |
| Antipsychotic | GC-TOF | 267665 | 0.54 |
| Statins | GC-TOF | 268306 | 0.54 |
| Anxioytic | GC-TOF | 299416 | 0.54 |
| Corticosteroids | GC-TOF | 309641 | 0.54 |
| Antidepressant | GC-TOF | ERYTHROSE | 0.54 |
| Anxioytic | GC-TOF | STEARIC_ACID | 0.54 |
| Antidepressant | LC-ECA | 11_36_75 | 0.54 |
| Statins | LC-ECA | 8_76_933 | 0.54 |
| Anxioytic | LC-ECA | 8_89_433 | 0.54 |
| Antipsychotic | GC-TOF | 199777 | 0.55 |
| Antipsychotic | GC-TOF | 200556 | 0.55 |
| Antidepressant | GC-TOF | 202599 | 0.55 |
| Statins | GC-TOF | 212274 | 0.55 |
| Antipsychotic | GC-TOF | 218597 | 0.55 |
| Anxioytic | GC-TOF | 224322 | 0.55 |
| Antidepressant | GC-TOF | 226853 | 0.55 |
| Anxioytic | GC-TOF | 227655 | 0.55 |
| Antipsychotic | GC-TOF | 267884 | 0.55 |
| Antidepressant | GC-TOF | 271416 | 0.55 |
| Corticosteroids | GC-TOF | 306152 | 0.55 |
| Antidepressant | GC-TOF | 308328 | 0.55 |
| Antidepressant | GC-TOF | 309545 | 0.55 |
| Statins | GC-TOF | ACONITIC_ACID | 0.55 |
| Statins | GC-TOF | CITRAMALATE | 0.55 |
| Statins | GC-TOF | GLUTAMIC_ACID | 0.55 |
| Statins | GC-TOF | PHOSPHATE | 0.55 |
| Antidepressant | GC-TOF | TYROSINE | 0.55 |
| Statins | LC-ECA | I3AA | 0.55 |
| Antipsychotic | LC-ECA | 11_60_917 | 0.55 |
| Anxioytic | LC-ECA | 13_38_49 | 0.55 |
| Statins | LC-ECA | 4_22_117 | 0.55 |
| Statins | LC-ECA | 4HBAC | 0.55 |
| Antidepressant | LC-ECA | 4HPLA | 0.55 |
| Statins | LC-ECA | 5_24_483 | 0.55 |
| Antipsychotic | GC-TOF | 200541 | 0.56 |
| Statins | GC-TOF | 202885 | 0.56 |
| Antidepressant | GC-TOF | 208686 | 0.56 |
| Statins | GC-TOF | 225863 | 0.56 |
| Corticosteroids | GC-TOF | 268709 | 0.56 |
| Statins | GC-TOF | 280940 | 0.56 |
| Statins | GC-TOF | 289055 | 0.56 |
| Antipsychotic | GC-TOF | 312977 | 0.56 |
| Antipsychotic | GC-TOF | ALPHA_KETOGLUTARIC_ACID | 0.56 |
| Statins | GC-TOF | GLUCOSE | 0.56 |
| Antidepressant | GC-TOF | GLUTAMINE_DEH_{—} | 0.56 |
| Corticosteroids | GC-TOF | GLYCINE | 0.56 |
| Anxioytic | GC-TOF | PHOSPHATE | 0.56 |
| Corticosteroids | GC-TOF | PHOSPHATE | 0.56 |
| Antipsychotic | GC-TOF | PHOSPHOETHANOLAMINE | 0.56 |
| Antipsychotic | GC-TOF | PROLINE | 0.56 |
| Antipsychotic | GC-TOF | THREONINE | 0.56 |
| Antipsychotic | GC-TOF | XYLULOSE_NIST | 0.56 |
| Anxioytic | LC-ECA | 12_50_183 | 0.56 |
| Antipsychotic | LC-ECA | 4_22_117 | 0.56 |
| Antipsychotic | GC-TOF | 206308 | 0.57 |
| Antipsychotic | GC-TOF | 216428 | 0.57 |
| Anxioytic | GC-TOF | 217870 | 0.57 |
| Antipsychotic | GC-TOF | 225548 | 0.57 |
| Anxioytic | GC-TOF | 225548 | 0.57 |
| Antidepressant | GC-TOF | 228528 | 0.57 |
| Statins | GC-TOF | 231099 | 0.57 |
| Anxioytic | GC-TOF | 241097 | 0.57 |
| Antipsychotic | GC-TOF | 241920 | 0.57 |
| Anxioytic | GC-TOF | 280573 | 0.57 |
| Antidepressant | GC-TOF | 306156 | 0.57 |
| Statins | GC-TOF | 306157 | 0.57 |
| Anxioytic | GC-TOF | FUCOSE | 0.57 |
| Anxioytic | GC-TOF | LACTIC_ACID | 0.57 |
| Antidepressant | GC-TOF | N_METHYLALANINE | 0.57 |
| Anxioytic | GC-TOF | ORNITHINE | 0.57 |
| Statins | GC-TOF | PHOSPHOETHANOLAMINE | 0.57 |
| Corticosteroids | GC-TOF | PUTRESCINE | 0.57 |
| Antidepressant | GC-TOF | RIBITOL | 0.57 |
| Statins | GC-TOF | SERINE | 0.57 |
| Corticosteroids | GC-TOF | TRYPTOPHAN | 0.57 |
| Antipsychotic | LC-ECA | GSH | 0.57 |
| Antidepressant | LC-ECA | 5_15_692 | 0.57 |
| Corticosteroids | LC-ECA | 8_14_983 | 0.57 |
| Corticosteroids | GC-TOF | 201005 | 0.58 |
| Anxioytic | GC-TOF | 223505 | 0.58 |
| Anxioytic | GC-TOF | 238467 | 0.58 |
| Antidepressant | GC-TOF | 240432 | 0.58 |
| Statins | GC-TOF | 299416 | 0.58 |
| Antidepressant | GC-TOF | 307889 | 0.58 |
| Corticosteroids | GC-TOF | 307889 | 0.58 |
| Statins | GC-TOF | 312308 | 0.58 |
| Anxioytic | GC-TOF | DIHYDROXYMALONIC_ACID_NIST | 0.58 |
| Anxioytic | GC-TOF | FUCOSE_RHAMNOSE_{—} | 0.58 |
| Antidepressant | GC-TOF | GLYCEROL_ALPHA_PHOSPHATE | 0.58 |
| Statins | GC-TOF | PSEUDO_URIDINE | 0.58 |
| Statins | LC-ECA | GR | 0.58 |
| Corticosteroids | LC-ECA | MHPG | 0.58 |
| Statins | LC-ECA | 15_68_542 | 0.58 |
| Anxioytic | GC-TOF | 2_DEOXYERYTHRITOL | 0.59 |
| Antidepressant | GC-TOF | 203235 | 0.59 |
| Statins | GC-TOF | 215978 | 0.59 |
| Antipsychotic | GC-TOF | 218710 | 0.59 |
| Antipsychotic | GC-TOF | 225863 | 0.59 |
| Antipsychotic | GC-TOF | 226303 | 0.59 |
| Statins | GC-TOF | 228557 | 0.59 |
| Anxioytic | GC-TOF | 301536 | 0.59 |
| Statins | GC-TOF | 303060 | 0.59 |
| Corticosteroids | GC-TOF | 309573 | 0.59 |
| Corticosteroids | GC-TOF | 310448 | 0.59 |
| Statins | GC-TOF | BETA_MANNOSYLGLYCERATE_MINOR_{—} | 0.59 |
| Antidepressant | GC-TOF | THREITOL_{—} | 0.59 |
| Antidepressant | GC-TOF | XANTHINE | 0.59 |
| Antipsychotic | LC-ECA | PXAN | 0.59 |
| Corticosteroids | LC-ECA | 14_34_25 | 0.59 |
| Antidepressant | GC-TOF | 199203 | 0.6 |
| Antipsychotic | GC-TOF | 202573 | 0.6 |
| Corticosteroids | GC-TOF | 213143 | 0.6 |
| Antidepressant | GC-TOF | 218513 | 0.6 |
| Statins | GC-TOF | 227597 | 0.6 |
| Anxioytic | GC-TOF | 231674 | 0.6 |
| Antipsychotic | GC-TOF | 299416 | 0.6 |
| Corticosteroids | GC-TOF | 3_HYDROXYPROPIONIC_ACID | 0.6 |
| Corticosteroids | GC-TOF | 301583 | 0.6 |
| Anxioytic | GC-TOF | IDONIC_ACID_NIST | 0.6 |
| Antidepressant | GC-TOF | PHENYLALANINE | 0.6 |
| Statins | GC-TOF | THREITOL_{—} | 0.6 |
| Statins | LC-ECA | ASA | 0.6 |
| Anxioytic | LC-ECA | HVA | 0.6 |
| Corticosteroids | LC-ECA | I3AA | 0.6 |
| Antipsychotic | LC-ECA | 9_33_1 | 0.6 |
| Statins | GC-TOF | 2_DEOXYERYTHRITOL | 0.61 |
| Antidepressant | GC-TOF | 208655 | 0.61 |
| Anxioytic | GC-TOF | 208655 | 0.61 |
| Antipsychotic | GC-TOF | 213697 | 0.61 |
| Antidepressant | GC-TOF | 228369 | 0.61 |
| Anxioytic | GC-TOF | 231056 | 0.61 |
| Antidepressant | GC-TOF | 241881 | 0.61 |
| Antipsychotic | GC-TOF | 268321 | 0.61 |
| Antidepressant | GC-TOF | 268709 | 0.61 |
| Anxioytic | GC-TOF | 281257 | 0.61 |
| Statins | GC-TOF | 288808 | 0.61 |
| Antidepressant | GC-TOF | 3_HYDROXYPROPIONIC_ACID | 0.61 |
| Antidepressant | GC-TOF | 303060 | 0.61 |
| Corticosteroids | GC-TOF | 306157 | 0.61 |
| Statins | GC-TOF | ACETOPHENONE_NIST | 0.61 |
| Statins | GC-TOF | ERYTHRITOL | 0.61 |
| Statins | GC-TOF | OXOPROLINE | 0.61 |
| Antidepressant | LC-ECA | KYN | 0.61 |
| Antidepressant | LC-ECA | 11_46_55 | 0.61 |
| Corticosteroids | LC-ECA | 4_32_592 | 0.61 |
| Corticosteroids | LC-ECA | 5_102_808 | 0.61 |
| Antipsychotic | LC-ECA | 5_24_483 | 0.61 |
| Antidepressant | LC-ECA | 8_89_433 | 0.61 |
| Corticosteroids | GC-TOF | 215978 | 0.62 |
| Antidepressant | GC-TOF | 218767 | 0.62 |
| Corticosteroids | GC-TOF | 225863 | 0.62 |
| Corticosteroids | GC-TOF | 227597 | 0.62 |
| Antipsychotic | GC-TOF | 228528 | 0.62 |
| Corticosteroids | GC-TOF | 231326 | 0.62 |
| Antipsychotic | GC-TOF | 231709 | 0.62 |
| Antidepressant | GC-TOF | 241097 | 0.62 |
| Antidepressant | GC-TOF | 301536 | 0.62 |
| Antipsychotic | GC-TOF | 309573 | 0.62 |
| Antidepressant | GC-TOF | 312448 | 0.62 |
| Antipsychotic | GC-TOF | GLUCONIC_ACID | 0.62 |
| Anxioytic | GC-TOF | INOSINE | 0.62 |
| Antidepressant | GC-TOF | METHYLHEXADECANOIC_ACID | 0.62 |
| Corticosteroids | GC-TOF | PROLINE | 0.62 |
| Corticosteroids | LC-ECA | 14_22_758 | 0.62 |
| Antidepressant | LC-ECA | 14_64_275 | 0.62 |
| Antipsychotic | GC-TOF | 202572 | 0.63 |
| Antipsychotic | GC-TOF | 218513 | 0.63 |
| Antidepressant | GC-TOF | 224551 | 0.63 |
| Antidepressant | GC-TOF | 226906 | 0.63 |
| Statins | GC-TOF | 238270 | 0.63 |
| Antidepressant | GC-TOF | 268306 | 0.63 |
| Antipsychotic | GC-TOF | 270407 | 0.63 |
| Antipsychotic | GC-TOF | 281907 | 0.63 |
| Statins | GC-TOF | 3_DEOXYPENTITOL_NIST | 0.63 |
| Statins | GC-TOF | 306159 | 0.63 |
| Antipsychotic | GC-TOF | 309538 | 0.63 |
| Anxioytic | GC-TOF | ARABINOSE | 0.63 |
| Statins | GC-TOF | CITRIC_ACID | 0.63 |
| Anxioytic | GC-TOF | GLUCOHEPTULOSE | 0.63 |
| Antipsychotic | GC-TOF | GLYCOLIC_ACID | 0.63 |
| Statins | GC-TOF | THREONINE | 0.63 |
| Statins | LC-ECA | MET | 0.63 |
| Corticosteroids | LC-ECA | URIC | 0.63 |
| Anxioytic | LC-ECA | 11_36_75 | 0.63 |
| Corticosteroids | LC-ECA | 8_93_65 | 0.63 |
| Anxioytic | GC-TOF | 200541 | 0.64 |
| Antidepressant | GC-TOF | 200595 | 0.64 |
| Corticosteroids | GC-TOF | 202573 | 0.64 |
| Antipsychotic | GC-TOF | 214426 | 0.64 |
| Anxioytic | GC-TOF | 223830 | 0.64 |
| Antipsychotic | GC-TOF | 228557 | 0.64 |
| Antipsychotic | GC-TOF | 228911 | 0.64 |
| Corticosteroids | GC-TOF | 231099 | 0.64 |
| Antidepressant | GC-TOF | 269625 | 0.64 |
| Corticosteroids | GC-TOF | GLUCONIC_ACID | 0.64 |
| Statins | GC-TOF | PHOSPHORIC_ACID | 0.64 |
| Corticosteroids | GC-TOF | SERINE | 0.64 |
| Corticosteroids | LC-ECA | 11_46_55 | 0.64 |
| Antidepressant | GC-TOF | 2_MONOSTEARIN_NIST | 0.65 |
| Antidepressant | GC-TOF | 216428 | 0.65 |
| Statins | GC-TOF | 218951 | 0.65 |
| Antipsychotic | GC-TOF | 219683 | 0.65 |
| Antidepressant | GC-TOF | 234015 | 0.65 |
| Anxioytic | GC-TOF | 281216 | 0.65 |
| Statins | GC-TOF | 293849 | 0.65 |
| Statins | GC-TOF | 306156 | 0.65 |
| Antidepressant | GC-TOF | ARABITOL | 0.65 |
| Anxioytic | GC-TOF | ERYTHRITOL | 0.65 |
| Antipsychotic | LC-ECA | ASA | 0.65 |
| Statins | LC-ECA | XANTH | 0.65 |
| Statins | GC-TOF | 200541 | 0.66 |
| Corticosteroids | GC-TOF | 200906 | 0.66 |
| Antipsychotic | GC-TOF | 208655 | 0.66 |
| Antipsychotic | GC-TOF | 212274 | 0.66 |
| Corticosteroids | GC-TOF | 224020 | 0.66 |
| Antidepressant | GC-TOF | 239954 | 0.66 |
| Antipsychotic | GC-TOF | 289055 | 0.66 |
| Statins | GC-TOF | 293848 | 0.66 |
| Antipsychotic | GC-TOF | 3_HYDROXY_3_METHYLGLUTARIC_ACID | 0.66 |
| Corticosteroids | GC-TOF | 301536 | 0.66 |
| Statins | GC-TOF | 309532 | 0.66 |
| Anxioytic | GC-TOF | 310010 | 0.66 |
| Anxioytic | GC-TOF | 312592 | 0.66 |
| Antipsychotic | GC-TOF | GLYCERIC_ACID | 0.66 |
| Antipsychotic | GC-TOF | PALMITIC_ACID | 0.66 |
| Statins | GC-TOF | XYLULOSE_NIST | 0.66 |
| Statins | LC-ECA | KYN | 0.66 |
| Antipsychotic | LC-ECA | 11_51_158 | 0.66 |
| Statins | LC-ECA | 13_19_492 | 0.66 |
| Anxioytic | LC-ECA | 15_77_017 | 0.66 |
| Antipsychotic | LC-ECA | 15_90_6 | 0.66 |
| Statins | GC-TOF | 200906 | 0.67 |
| Statins | GC-TOF | 201887 | 0.67 |
| Statins | GC-TOF | 213198 | 0.67 |
| Statins | GC-TOF | 223830 | 0.67 |
| Antidepressant | GC-TOF | 310010 | 0.67 |
| Statins | GC-TOF | ARABITOL | 0.67 |
| Anxioytic | GC-TOF | ASPARAGINE_DEH_{—} | 0.67 |
| Antidepressant | GC-TOF | GLYCINE | 0.67 |
| Statins | GC-TOF | ISOLEUCINE | 0.67 |
| Corticosteroids | GC-TOF | SUCROSE | 0.67 |
| Corticosteroids | LC-ECA | 13_92_333 | 0.67 |
| Antidepressant | GC-TOF | 202885 | 0.68 |
| Antidepressant | GC-TOF | 213143 | 0.68 |
| Corticosteroids | GC-TOF | 213193 | 0.68 |
| Corticosteroids | GC-TOF | 215494 | 0.68 |
| Antidepressant | GC-TOF | 222169 | 0.68 |
| Antipsychotic | GC-TOF | 231657 | 0.68 |
| Anxioytic | GC-TOF | 241920 | 0.68 |
| Antipsychotic | GC-TOF | 296108 | 0.68 |
| Statins | GC-TOF | 310448 | 0.68 |
| Antipsychotic | GC-TOF | PSEUDO_URIDINE | 0.68 |
| Anxioytic | LC-ECA | 14_34_25 | 0.68 |
| Statins | LC-ECA | 15_90_6 | 0.68 |
| Antipsychotic | LC-ECA | 5_102_808 | 0.68 |
| Statins | LC-ECA | 8_93_65 | 0.68 |
| Statins | GC-TOF | 199463 | 0.69 |
| Statins | GC-TOF | 203765 | 0.69 |
| Antipsychotic | GC-TOF | 224551 | 0.69 |
| Antipsychotic | GC-TOF | 231947 | 0.69 |
| Anxioytic | GC-TOF | 294129 | 0.69 |
| Antipsychotic | GC-TOF | 3_HYDROXYPROPIONIC_ACID | 0.69 |
| Statins | GC-TOF | 3_HYDROXYPROPIONIC_ACID | 0.69 |
| Antidepressant | GC-TOF | 310448 | 0.69 |
| Anxioytic | GC-TOF | ACONITIC_ACID | 0.69 |
| Anxioytic | GC-TOF | ASCORBIC_ACID | 0.69 |
| Anxioytic | GC-TOF | DEHYDROASCORBATE | 0.69 |
| Anxioytic | GC-TOF | PUTREANINE_NIST | 0.69 |
| Statins | GC-TOF | TRYPTOPHAN | 0.69 |
| Antidepressant | LC-ECA | 5HTP | 0.69 |
| Anxioytic | LC-ECA | 9_29_925 | 0.69 |
| Antipsychotic | GC-TOF | 208755 | 0.7 |
| Statins | GC-TOF | 218710 | 0.7 |
| Anxioytic | GC-TOF | 223535 | 0.7 |
| Anxioytic | GC-TOF | 228605 | 0.7 |
| Statins | GC-TOF | 231326 | 0.7 |
| Anxioytic | GC-TOF | 231544 | 0.7 |
| Statins | GC-TOF | 268709 | 0.7 |
| Statins | GC-TOF | 270351 | 0.7 |
| Antipsychotic | GC-TOF | 280573 | 0.7 |
| Antidepressant | GC-TOF | 308185 | 0.7 |
| Statins | GC-TOF | 309641 | 0.7 |
| Statins | GC-TOF | 310010 | 0.7 |
| Antipsychotic | GC-TOF | 312362 | 0.7 |
| Antipsychotic | GC-TOF | 312622 | 0.7 |
| Anxioytic | GC-TOF | ALPHA_KETOGLUTARIC_ACID | 0.7 |
| Antidepressant | GC-TOF | BETA_MANNOSYLGLYCERATE_MINOR | 0.7 |
| Anxioytic | GC-TOF | CYSTEINE | 0.7 |
| Statins | GC-TOF | GLUCOHEPTOSE | 0.7 |
| Corticosteroids | GC-TOF | GLUTAMIC_ACID | 0.7 |
| Antidepressant | GC-TOF | GLYCERIC_ACID | 0.7 |
| Antipsychotic | GC-TOF | THREONIC_ACID | 0.7 |
| Antidepressant | LC-ECA | GR | 0.7 |
| Antidepressant | LC-ECA | 12_52_75 | 0.7 |
| Statins | LC-ECA | 9_25_825 | 0.7 |
| Statins | GC-TOF | 2_DEOXYTETRONIC_ACID | 0.71 |
| Statins | GC-TOF | 2_HYDROXYBUTANOIC_ACID | 0.71 |
| Corticosteroids | GC-TOF | 2_HYDROXYVALERIC_ACID | 0.71 |
| Statins | GC-TOF | 200556 | 0.71 |
| Antidepressant | GC-TOF | 218951 | 0.71 |
| Antidepressant | GC-TOF | 224635 | 0.71 |
| Antipsychotic | GC-TOF | 238270 | 0.71 |
| Statins | GC-TOF | 268579 | 0.71 |
| Antidepressant | GC-TOF | 280573 | 0.71 |
| Antidepressant | GC-TOF | 293848 | 0.71 |
| Antidepressant | GC-TOF | 3_HYDROXY_3_METHYLGLUTARIC_ACID | 0.71 |
| Anxioytic | GC-TOF | CHOLESTEROL_{—} | 0.71 |
| Antidepressant | GC-TOF | MALTOSE_1 | 0.71 |
| Antidepressant | LC-ECA | MET | 0.71 |
| Antidepressant | LC-ECA | TRP | 0.71 |
| Corticosteroids | LC-ECA | 9_33_1 | 0.71 |
| Antipsychotic | GC-TOF | 2_HYDROXYVALERIC_ACID | 0.72 |
| Antipsychotic | GC-TOF | 203235 | 0.72 |
| Corticosteroids | GC-TOF | 213697 | 0.72 |
| Antidepressant | GC-TOF | 226303 | 0.72 |
| Corticosteroids | GC-TOF | 228147 | 0.72 |
| Statins | GC-TOF | 231709 | 0.72 |
| Corticosteroids | GC-TOF | 232485 | 0.72 |
| Antidepressant | GC-TOF | 267649 | 0.72 |
| Corticosteroids | GC-TOF | 312679 | 0.72 |
| Anxioytic | GC-TOF | ALANINE | 0.72 |
| Antipsychotic | GC-TOF | CITRAMALATE | 0.72 |
| Antipsychotic | GC-TOF | DIHYDROXYMALONIC_ACID_NIST | 0.72 |
| Antipsychotic | GC-TOF | ENOLPYRUVATE_NIST | 0.72 |
| Antidepressant | GC-TOF | FUCOSE | 0.72 |
| Antidepressant | GC-TOF | PANTOTHENIC_ACID | 0.72 |
| Statins | LC-ECA | TYR | 0.72 |
| Statins | LC-ECA | 11_51_158 | 0.72 |
| Anxioytic | LC-ECA | 13_74_392 | 0.72 |
| Antidepressant | LC-ECA | 4_22_117 | 0.72 |
| Statins | GC-TOF | 199553 | 0.73 |
| Statins | GC-TOF | 2_KETOISOCAPROIC_ACID | 0.73 |
| Anxioytic | GC-TOF | 202091 | 0.73 |
| Antipsychotic | GC-TOF | 204318 | 0.73 |
| Anxioytic | GC-TOF | 210286 | 0.73 |
| Statins | GC-TOF | 218513 | 0.73 |
| Statins | GC-TOF | 221597 | 0.73 |
| Statins | GC-TOF | 224635 | 0.73 |
| Anxioytic | GC-TOF | 232485 | 0.73 |
| Statins | GC-TOF | 234580 | 0.73 |
| Corticosteroids | GC-TOF | 234622 | 0.73 |
| Statins | GC-TOF | 308185 | 0.73 |
| Statins | GC-TOF | 312592 | 0.73 |
| Corticosteroids | GC-TOF | ACONITIC_ACID | 0.73 |
| Statins | GC-TOF | CREATININE | 0.73 |
| Statins | GC-TOF | GLYCEROL_ALPHA_PHOSPHATE | 0.73 |
| Antidepressant | GC-TOF | ISOTHREONIC_ACID | 0.73 |
| Anxioytic | GC-TOF | SUCROSE | 0.73 |
| Anxioytic | GC-TOF | XYLITOL | 0.73 |
| Anxioytic | LC-ECA | 12_94_5 | 0.73 |
| Antipsychotic | LC-ECA | 13_38_49 | 0.73 |
| Corticosteroids | LC-ECA | 13_44_608 | 0.73 |
| Corticosteroids | LC-ECA | 8_76_933 | 0.73 |
| Antipsychotic | LC-ECA | 9_29_925 | 0.73 |
| Antidepressant | GC-TOF | 200906 | 0.74 |
| Antidepressant | GC-TOF | 202091 | 0.74 |
| Antidepressant | GC-TOF | 211935 | 0.74 |
| Corticosteroids | GC-TOF | 217870 | 0.74 |
| Antidepressant | GC-TOF | 223830 | 0.74 |
| Antipsychotic | GC-TOF | 235414 | 0.74 |
| Antipsychotic | GC-TOF | 280546 | 0.74 |
| Statins | GC-TOF | 301825 | 0.74 |
| Anxioytic | GC-TOF | 309573 | 0.74 |
| Corticosteroids | GC-TOF | 312289 | 0.74 |
| Corticosteroids | GC-TOF | IDONIC_ACID_NIST | 0.74 |
| Antidepressant | GC-TOF | MYRISTIC_ACID | 0.74 |
| Statins | GC-TOF | VALINE | 0.74 |
| Corticosteroids | LC-ECA | 13_86_8 | 0.74 |
| Antidepressant | LC-ECA | 8_82_917 | 0.74 |
| Antidepressant | LC-ECA | 8_93_65 | 0.74 |
| Antidepressant | LC-ECA | 9_33_1 | 0.74 |
| Antipsychotic | GC-TOF | 199553 | 0.75 |
| Antidepressant | GC-TOF | 200541 | 0.75 |
| Anxioytic | GC-TOF | 218951 | 0.75 |
| Corticosteroids | GC-TOF | 226851 | 0.75 |
| Antidepressant | GC-TOF | 232604 | 0.75 |
| Corticosteroids | GC-TOF | 241881 | 0.75 |
| Antipsychotic | GC-TOF | 268709 | 0.75 |
| Corticosteroids | GC-TOF | 306156 | 0.75 |
| Antidepressant | GC-TOF | 309573 | 0.75 |
| Statins | GC-TOF | ALPHA_KETOGLUTARIC_ACID | 0.75 |
| Statins | GC-TOF | RIBITOL | 0.75 |
| Antidepressant | GC-TOF | THREONINE | 0.75 |
| Antidepressant | GC-TOF | THYMINE | 0.75 |
| Statins | LC-ECA | VMA | 0.75 |
| Corticosteroids | LC-ECA | 13_18_475 | 0.75 |
| Statins | LC-ECA | 13_86_8 | 0.75 |
| Antidepressant | LC-ECA | 15_90_6 | 0.75 |
| Statins | GC-TOF | 308328 | 0.76 |
| Antipsychotic | GC-TOF | CITRIC_ACID | 0.76 |
| Statins | GC-TOF | HEXURONIC_ACID | 0.76 |
| Statins | GC-TOF | PHENYLALANINE | 0.76 |
| Corticosteroids | GC-TOF | RIBITOL | 0.76 |
| Anxioytic | GC-TOF | TAURINE | 0.76 |
| Corticosteroids | LC-ECA | 12_41_200 | 0.76 |
| Corticosteroids | LC-ECA | 4HPLA | 0.76 |
| Antidepressant | LC-ECA | 9_29_34 | 0.76 |
| Anxioytic | GC-TOF | 199463 | 0.77 |
| Antidepressant | GC-TOF | 2_DEOXYERYTHRITOL | 0.77 |
| Corticosteroids | GC-TOF | 206308 | 0.77 |
| Anxioytic | GC-TOF | 224551 | 0.77 |
| Statins | GC-TOF | ASCORBIC_ACID | 0.77 |
| Statins | GC-TOF | CONDURITOL_BETA_EPOXIDE | 0.77 |
| Antidepressant | GC-TOF | ETHANOLAMINE | 0.77 |
| Antidepressant | GC-TOF | GLUTAMIC_ACID | 0.77 |
| Anxioytic | GC-TOF | GLYCEROL_ALPHA_PHOSPHATE | 0.77 |
| Antidepressant | GC-TOF | INULOBIOSE_2 | 0.77 |
| Anxioytic | GC-TOF | PSEUDO_URIDINE | 0.77 |
| Antipsychotic | LC-ECA | 14_34_25 | 0.77 |
| Anxioytic | LC-ECA | 8_76_933 | 0.77 |
| Antidepressant | LC-ECA | 9_20_858 | 0.77 |
| Statins | GC-TOF | 199777 | 0.78 |
| Antipsychotic | GC-TOF | 2_DEOXYERYTHRITOL | 0.78 |
| Antipsychotic | GC-TOF | 215494 | 0.78 |
| Corticosteroids | GC-TOF | 218597 | 0.78 |
| Anxioytic | GC-TOF | 222169 | 0.78 |
| Antipsychotic | GC-TOF | 223830 | 0.78 |
| Anxioytic | GC-TOF | 227270 | 0.78 |
| Antidepressant | GC-TOF | 231657 | 0.78 |
| Statins | GC-TOF | 267737 | 0.78 |
| Corticosteroids | GC-TOF | 268365 | 0.78 |
| Antidepressant | GC-TOF | 281216 | 0.78 |
| Statins | GC-TOF | 294129 | 0.78 |
| Statins | GC-TOF | 296108 | 0.78 |
| Corticosteroids | GC-TOF | 309532 | 0.78 |
| Corticosteroids | GC-TOF | 309934 | 0.78 |
| Antipsychotic | GC-TOF | ERYTHRITOL | 0.78 |
| Antidepressant | GC-TOF | FUCOSE_RHAMNOSE_{—} | 0.78 |
| Anxioytic | GC-TOF | OXOPROLINE | 0.78 |
| Corticosteroids | LC-ECA | 11_60_917 | 0.78 |
| Statins | LC-ECA | 12_94_5 | 0.78 |
| Corticosteroids | LC-ECA | 14_75_608 | 0.78 |
| Statins | GC-TOF | 227387 | 0.79 |
| Antidepressant | GC-TOF | 227582 | 0.79 |
| Antipsychotic | GC-TOF | 227655 | 0.79 |
| Antidepressant | GC-TOF | 3_DEOXYPENTITOL_NIST | 0.79 |
| Antidepressant | GC-TOF | 309532 | 0.79 |
| Statins | GC-TOF | 312448 | 0.79 |
| Corticosteroids | GC-TOF | 312977 | 0.79 |
| Statins | GC-TOF | CHOLESTEROL_{—} | 0.79 |
| Antipsychotic | GC-TOF | CONDURITOL_BETA_EPOXIDE | 0.79 |
| Statins | GC-TOF | DIHYDROXYMALONIC_ACID_NIST | 0.79 |
| Statins | GC-TOF | FRUCTOSE | 0.79 |
| Statins | GC-TOF | GLUCONIC_ACID | 0.79 |
| Antipsychotic | GC-TOF | LACTIC_ACID | 0.79 |
| Anxioytic | GC-TOF | LYSINE | 0.79 |
| Statins | GC-TOF | ORNITHINE | 0.79 |
| Antidepressant | GC-TOF | XYLOSE | 0.79 |
| Corticosteroids | LC-ECA | 2HPAC | 0.79 |
| Antipsychotic | LC-ECA | 5HTP | 0.79 |
| Anxioytic | GC-TOF | 199777 | 0.8 |
| Corticosteroids | GC-TOF | 2_KETOISOCAPROIC_ACID | 0.8 |
| Statins | GC-TOF | 202091 | 0.8 |
| Anxioytic | GC-TOF | 204318 | 0.8 |
| Corticosteroids | GC-TOF | 219683 | 0.8 |
| Anxioytic | GC-TOF | 224635 | 0.8 |
| Antipsychotic | GC-TOF | 234015 | 0.8 |
| Anxioytic | GC-TOF | 234622 | 0.8 |
| Antidepressant | GC-TOF | 267884 | 0.8 |
| Statins | GC-TOF | 281216 | 0.8 |
| Anxioytic | GC-TOF | 301583 | 0.8 |
| Antipsychotic | GC-TOF | 306159 | 0.8 |
| Anxioytic | GC-TOF | CREATININE | 0.8 |
| Antidepressant | GC-TOF | HEXURONIC_ACID | 0.8 |
| Statins | GC-TOF | ISOCITRIC_ACID | 0.8 |
| Corticosteroids | LC-ECA | 13_38_49 | 0.8 |
| Statins | GC-TOF | 200490 | 0.81 |
| Antipsychotic | GC-TOF | 200906 | 0.81 |
| Corticosteroids | GC-TOF | 203235 | 0.81 |
| Antipsychotic | GC-TOF | 203765 | 0.81 |
| Corticosteroids | GC-TOF | 217783 | 0.81 |
| Corticosteroids | GC-TOF | 227655 | 0.81 |
| Statins | GC-TOF | 231544 | 0.81 |
| Statins | GC-TOF | 233005 | 0.81 |
| Corticosteroids | GC-TOF | 241097 | 0.81 |
| Statins | GC-TOF | 267665 | 0.81 |
| Corticosteroids | GC-TOF | 268321 | 0.81 |
| Antipsychotic | GC-TOF | 268365 | 0.81 |
| Anxioytic | GC-TOF | 312308 | 0.81 |
| Statins | GC-TOF | LACTIC_ACID | 0.81 |
| Statins | GC-TOF | N_METHYLALANINE | 0.81 |
| Corticosteroids | GC-TOF | PELARGONIC_ACID | 0.81 |
| Anxioytic | GC-TOF | PHENYLALANINE | 0.81 |
| Antidepressant | LC-ECA | PXAN | 0.81 |
| Antidepressant | LC-ECA | 15_68_542 | 0.81 |
| Statins | GC-TOF | 200595 | 0.82 |
| Statins | GC-TOF | 211935 | 0.82 |
| Antidepressant | GC-TOF | 217870 | 0.82 |
| Antipsychotic | GC-TOF | 224322 | 0.82 |
| Corticosteroids | GC-TOF | 227582 | 0.82 |
| Antipsychotic | GC-TOF | 228147 | 0.82 |
| Corticosteroids | GC-TOF | 228605 | 0.82 |
| Corticosteroids | GC-TOF | 228612 | 0.82 |
| Antipsychotic | GC-TOF | 228885 | 0.82 |
| Anxioytic | GC-TOF | 267737 | 0.82 |
| Antidepressant | GC-TOF | 268365 | 0.82 |
| Statins | GC-TOF | 280546 | 0.82 |
| Statins | GC-TOF | 280573 | 0.82 |
| Anxioytic | GC-TOF | 293848 | 0.82 |
| Antipsychotic | GC-TOF | 309873 | 0.82 |
| Statins | GC-TOF | GLYCINE | 0.82 |
| Antidepressant | GC-TOF | LYSINE | 0.82 |
| Anxioytic | GC-TOF | PROLINE | 0.82 |
| Corticosteroids | LC-ECA | 5_40_292 | 0.82 |
| Corticosteroids | GC-TOF | 224551 | 0.83 |
| Antidepressant | GC-TOF | 231850 | 0.83 |
| Statins | GC-TOF | 232604 | 0.83 |
| Statins | GC-TOF | 234622 | 0.83 |
| Antipsychotic | GC-TOF | 312448 | 0.83 |
| Corticosteroids | GC-TOF | 312622 | 0.83 |
| Antipsychotic | GC-TOF | ACETOPHENONE_NIST | 0.83 |
| Antipsychotic | GC-TOF | MALTOSE_1 | 0.83 |
| Corticosteroids | GC-TOF | METHIONINE | 0.83 |
| Antipsychotic | GC-TOF | PELARGONIC_ACID | 0.83 |
| Anxioytic | GC-TOF | PHOSPHOETHANOLAMINE | 0.83 |
| Antipsychotic | LC-ECA | KYN | 0.83 |
| Antidepressant | GC-TOF | 2_MONOPALMI | 0.84 |
| Antipsychotic | GC-TOF | 208686 | 0.84 |
| Antipsychotic | GC-TOF | 227387 | 0.84 |
| Statins | GC-TOF | 228612 | 0.84 |
| Statins | GC-TOF | 268365 | 0.84 |
| Statins | GC-TOF | 270407 | 0.84 |
| Corticosteroids | GC-TOF | 309788 | 0.84 |
| Antidepressant | GC-TOF | CREATININE | 0.84 |
| Antipsychotic | GC-TOF | ERYTHROSE | 0.84 |
| Antidepressant | GC-TOF | IDONIC_ACID_NIST | 0.84 |
| Antipsychotic | GC-TOF | INOSINE | 0.84 |
| Antidepressant | GC-TOF | ISOLEUCINE | 0.84 |
| Corticosteroids | GC-TOF | ISOLEUCINE | 0.84 |
| Antidepressant | GC-TOF | PELARGONIC_ACID | 0.84 |
| Statins | GC-TOF | PUTRESCINE | 0.84 |
| Statins | GC-TOF | RIBOSE | 0.84 |
| Antipsychotic | GC-TOF | STEARIC_ACID | 0.84 |
| Antidepressant | GC-TOF | TRYPTOPHAN | 0.84 |
| Antipsychotic | LC-ECA | 13_19_492 | 0.84 |
| Anxioytic | LC-ECA | 4_32_592 | 0.84 |
| Antipsychotic | GC-TOF | 199463 | 0.85 |
| Antipsychotic | GC-TOF | 204344 | 0.85 |
| Antipsychotic | GC-TOF | 213182 | 0.85 |
| Corticosteroids | GC-TOF | 213227 | 0.85 |
| Anxioytic | GC-TOF | 224020 | 0.85 |
| Corticosteroids | GC-TOF | 224035 | 0.85 |
| Antidepressant | GC-TOF | 231056 | 0.85 |
| Antipsychotic | GC-TOF | 231056 | 0.85 |
| Statins | GC-TOF | 235414 | 0.85 |
| Antipsychotic | GC-TOF | 3_HYDROXYBUTANOIC_ACID | 0.85 |
| Antipsychotic | GC-TOF | CREATININE | 0.85 |
| Antipsychotic | GC-TOF | GLYCEROL | 0.85 |
| Statins | GC-TOF | INOSINE | 0.85 |
| Antipsychotic | LC-ECA | I3AA | 0.85 |
| Corticosteroids | LC-ECA | 12_52_75 | 0.85 |
| Statins | LC-ECA | 13_84_975 | 0.85 |
| Statins | LC-ECA | 14_22_758 | 0.85 |
| Corticosteroids | LC-ECA | 9_25_825 | 0.85 |
| Antidepressant | GC-TOF | 199806 | 0.86 |
| Statins | GC-TOF | 199806 | 0.86 |
| Statins | GC-TOF | 204425 | 0.86 |
| Antidepressant | GC-TOF | 224849 | 0.86 |
| Corticosteroids | GC-TOF | 228528 | 0.86 |
| Corticosteroids | GC-TOF | 228911 | 0.86 |
| Anxioytic | GC-TOF | 231326 | 0.86 |
| Anxioytic | GC-TOF | 233340 | 0.86 |
| Antidepressant | GC-TOF | 306159 | 0.86 |
| Antidepressant | GC-TOF | GLUCONIC_ACID | 0.86 |
| Anxioytic | GC-TOF | GLYCINE | 0.86 |
| Antidepressant | GC-TOF | LEUCINE | 0.86 |
| Corticosteroids | LC-ECA | GR | 0.86 |
| Corticosteroids | LC-ECA | 5_24_483 | 0.86 |
| Antipsychotic | GC-TOF | 201005 | 0.87 |
| Statins | GC-TOF | 208686 | 0.87 |
| Antipsychotic | GC-TOF | 213227 | 0.87 |
| Statins | GC-TOF | 223505 | 0.87 |
| Anxioytic | GC-TOF | 238149 | 0.87 |
| Corticosteroids | GC-TOF | 281907 | 0.87 |
| Antidepressant | GC-TOF | 310831 | 0.87 |
| Antipsychotic | GC-TOF | 312308 | 0.87 |
| Corticosteroids | GC-TOF | CITRIC_ACID | 0.87 |
| Antidepressant | GC-TOF | GLYCEROL | 0.87 |
| Antidepressant | GC-TOF | INOSINE | 0.87 |
| Antipsychotic | GC-TOF | METHYLHEXADECANOIC_ACID | 0.87 |
| Antidepressant | GC-TOF | SUCCINIC_ACID | 0.87 |
| Antidepressant | LC-ECA | 4HPAC | 0.87 |
| Anxioytic | GC-TOF | 206308 | 0.88 |
| Anxioytic | GC-TOF | 226303 | 0.88 |
| Corticosteroids | GC-TOF | 226935 | 0.88 |
| Antipsychotic | GC-TOF | 231326 | 0.88 |
| Anxioytic | GC-TOF | 242417 | 0.88 |
| Anxioytic | GC-TOF | 267650 | 0.88 |
| Statins | GC-TOF | 281257 | 0.88 |
| Corticosteroids | GC-TOF | 301325 | 0.88 |
| Statins | GC-TOF | 301583 | 0.88 |
| Statins | GC-TOF | 309642 | 0.88 |
| Corticosteroids | GC-TOF | ACETOPHENONE_NIST | 0.88 |
| Corticosteroids | GC-TOF | ALANINE | 0.88 |
| Corticosteroids | GC-TOF | ASPARAGINE_DEH_{—} | 0.88 |
| Statins | GC-TOF | ASPARTIC_ACID | 0.88 |
| Statins | GC-TOF | SORBITOL | 0.88 |
| Antidepressant | LC-ECA | 13_18_475 | 0.88 |
| Anxioytic | GC-TOF | 2_DEOXYRIBONIC_ACID | 0.89 |
| Statins | GC-TOF | 228605 | 0.89 |
| Antidepressant | GC-TOF | 232485 | 0.89 |
| Antipsychotic | GC-TOF | 267675 | 0.89 |
| Antipsychotic | GC-TOF | 270351 | 0.89 |
| Antipsychotic | GC-TOF | 310010 | 0.89 |
| Antidepressant | GC-TOF | GLUCOHEPTULOSE | 0.89 |
| Corticosteroids | GC-TOF | GLUCOSE | 0.89 |
| Antipsychotic | GC-TOF | IDONIC_ACID_NIST | 0.89 |
| Anxioytic | GC-TOF | INULOBIOSE_2 | 0.89 |
| Antidepressant | GC-TOF | OXOPROLINE | 0.89 |
| Antidepressant | GC-TOF | PSEUDO_URIDINE | 0.89 |
| Anxioytic | GC-TOF | TYROSINE | 0.89 |
| Corticosteroids | LC-ECA | TRP | 0.89 |
| Antipsychotic | LC-ECA | 15_77_017 | 0.89 |
| Corticosteroids | LC-ECA | 15_77_017 | 0.89 |
| Statins | GC-TOF | 202572 | 0.9 |
| Antidepressant | GC-TOF | 206308 | 0.9 |
| Antipsychotic | GC-TOF | 218787 | 0.9 |
| Statins | GC-TOF | 228147 | 0.9 |
| Antipsychotic | GC-TOF | 231097 | 0.9 |
| Corticosteroids | GC-TOF | 267816 | 0.9 |
| Antidepressant | GC-TOF | 268321 | 0.9 |
| Anxioytic | GC-TOF | 293097 | 0.9 |
| Anxioytic | GC-TOF | 293849 | 0.9 |
| Antipsychotic | GC-TOF | 300379 | 0.9 |
| Anxioytic | GC-TOF | 308185 | 0.9 |
| Statins | GC-TOF | 310831 | 0.9 |
| Antipsychotic | GC-TOF | 4_HYDROXYBUTYRIC_ACID | 0.9 |
| Antipsychotic | GC-TOF | GLUTAMIC_ACID | 0.9 |
| Antipsychotic | GC-TOF | HEXURONIC_ACID | 0.9 |
| Corticosteroids | GC-TOF | MALTOSE_1 | 0.9 |
| Antidepressant | GC-TOF | MANNITOL | 0.9 |
| Anxioytic | GC-TOF | METHIONINE | 0.9 |
| Antidepressant | GC-TOF | PUTREANINE_NIST | 0.9 |
| Corticosteroids | GC-TOF | SORBITOL | 0.9 |
| Corticosteroids | LC-ECA | 12_50_183 | 0.9 |
| Antidepressant | LC-ECA | 13_19_492 | 0.9 |
| Antipsychotic | LC-ECA | 4HPAC | 0.9 |
| Antipsychotic | LC-ECA | 9_20_858 | 0.9 |
| Statins | GC-TOF | 217870 | 0.91 |
| Statins | GC-TOF | 231056 | 0.91 |
| Antidepressant | GC-TOF | 267737 | 0.91 |
| Corticosteroids | GC-TOF | LEUCINE | 0.91 |
| Antipsychotic | GC-TOF | ORNITHINE | 0.91 |
| Corticosteroids | GC-TOF | ORNITHINE | 0.91 |
| Antipsychotic | GC-TOF | OXOPROLINE | 0.91 |
| Corticosteroids | GC-TOF | PUTREANINE_NIST | 0.91 |
| Corticosteroids | GC-TOF | TYROSINE | 0.91 |
| Antidepressant | LC-ECA | I3PA | 0.91 |
| Antipsychotic | LC-ECA | 5HIAA | 0.91 |
| Corticosteroids | GC-TOF | 199777 | 0.92 |
| Corticosteroids | GC-TOF | 210168 | 0.92 |
| Statins | GC-TOF | 217783 | 0.92 |
| Statins | GC-TOF | 228885 | 0.92 |
| Corticosteroids | GC-TOF | 280546 | 0.92 |
| Antipsychotic | GC-TOF | 301583 | 0.92 |
| Antipsychotic | GC-TOF | 309545 | 0.92 |
| Antipsychotic | GC-TOF | BENZOIC_ACID | 0.92 |
| Corticosteroids | GC-TOF | DEHYDROASCORBATE | 0.92 |
| Statins | GC-TOF | GLUTAMINE_DEH_{—} | 0.92 |
| Statins | GC-TOF | MANNITOL | 0.92 |
| Statins | LC-ECA | 14_34_25 | 0.92 |
| Corticosteroids | LC-ECA | 8_82_917 | 0.92 |
| Corticosteroids | LC-ECA | XAN | 0.92 |
| Corticosteroids | GC-TOF | 214426 | 0.93 |
| Statins | GC-TOF | 215494 | 0.93 |
| Anxioytic | GC-TOF | 229164 | 0.93 |
| Antipsychotic | GC-TOF | 234580 | 0.93 |
| Antipsychotic | GC-TOF | 267816 | 0.93 |
| Corticosteroids | GC-TOF | 268306 | 0.93 |
| Statins | GC-TOF | 268321 | 0.93 |
| Antipsychotic | GC-TOF | 288019 | 0.93 |
| Antipsychotic | GC-TOF | 288808 | 0.93 |
| Anxioytic | GC-TOF | 312362 | 0.93 |
| Antidepressant | GC-TOF | 4_HYDROXYBUTYRIC_ACID | 0.93 |
| Antipsychotic | GC-TOF | ASPARAGINE_DEH_{—} | 0.93 |
| Corticosteroids | GC-TOF | ENOLPYRUVATE_NIST | 0.93 |
| Antipsychotic | GC-TOF | INOSITOL_ALLO_{—} | 0.93 |
| Statins | GC-TOF | LYSINE | 0.93 |
| Anxioytic | GC-TOF | MONOPALMITIN_1_GLYCERIDE | 0.93 |
| Anxioytic | GC-TOF | THREONIC_ACID | 0.93 |
| Statins | GC-TOF | THYMINE | 0.93 |
| Statins | LC-ECA | URIC | 0.93 |
| Corticosteroids | LC-ECA | 4HPAC | 0.93 |
| Anxioytic | GC-TOF | 201887 | 0.94 |
| Statins | GC-TOF | 202573 | 0.94 |
| Statins | GC-TOF | 210168 | 0.94 |
| Antidepressant | GC-TOF | 224020 | 0.94 |
| Antipsychotic | GC-TOF | 231099 | 0.94 |
| Corticosteroids | GC-TOF | 233340 | 0.94 |
| Antipsychotic | GC-TOF | 293849 | 0.94 |
| Antipsychotic | GC-TOF | 309532 | 0.94 |
| Antipsychotic | GC-TOF | 312902 | 0.94 |
| Statins | GC-TOF | ENOLPYRUVATE_NIST | 0.94 |
| Statins | GC-TOF | GLYCOLIC_ACID | 0.94 |
| Statins | GC-TOF | INOSITOL_ALLO_{—} | 0.94 |
| Statins | GC-TOF | INULOBIOSE_2 | 0.94 |
| Antipsychotic | GC-TOF | ISOCITRIC_ACID | 0.94 |
| Corticosteroids | GC-TOF | ISOCITRIC_ACID | 0.94 |
| Statins | GC-TOF | MALTOSE_1 | 0.94 |
| Statins | GC-TOF | PUTREANINE_NIST | 0.94 |
| Antidepressant | GC-TOF | SORBITOL | 0.94 |
| Statins | GC-TOF | TAURINE | 0.94 |
| Statins | GC-TOF | TYROSINE | 0.94 |
| Antidepressant | GC-TOF | VALINE | 0.94 |
| Anxioytic | LC-ECA | HX | 0.94 |
| Statins | LC-ECA | 2HPAC | 0.94 |
| Antipsychotic | GC-TOF | 1_MONOSTEARIN | 0.95 |
| Statins | GC-TOF | 2_DEOXYRIBONIC_ACID | 0.95 |
| Anxioytic | GC-TOF | 2_HYDROXYVALERIC_ACID | 0.95 |
| Anxioytic | GC-TOF | 213182 | 0.95 |
| Statins | GC-TOF | 213697 | 0.95 |
| Corticosteroids | GC-TOF | 231056 | 0.95 |
| Anxioytic | GC-TOF | 231850 | 0.95 |
| Statins | GC-TOF | 232485 | 0.95 |
| Corticosteroids | GC-TOF | 268313 | 0.95 |
| Antipsychotic | GC-TOF | 296106 | 0.95 |
| Statins | GC-TOF | METHIONINE | 0.95 |
| Antidepressant | LC-ECA | 14_75_608 | 0.95 |
| Anxioytic | LC-ECA | 14_75_608 | 0.95 |
| Corticosteroids | LC-ECA | 8_28_508 | 0.95 |
| Statins | GC-TOF | 202599 | 0.96 |
| Anxioytic | GC-TOF | 204344 | 0.96 |
| Statins | GC-TOF | 214426 | 0.96 |
| Antipsychotic | GC-TOF | 233340 | 0.96 |
| Corticosteroids | GC-TOF | 267650 | 0.96 |
| Antipsychotic | GC-TOF | 268579 | 0.96 |
| Statins | GC-TOF | 301536 | 0.96 |
| Corticosteroids | GC-TOF | INOSINE | 0.96 |
| Antipsychotic | GC-TOF | MONOPALMITIN_1_GLYCERIDE | 0.96 |
| Anxioytic | GC-TOF | PANTOTHENIC_ACID | 0.96 |
| Antipsychotic | GC-TOF | SALICYLALDEHYDE | 0.96 |
| Antidepressant | LC-ECA | TYR | 0.96 |
| Antidepressant | LC-ECA | 13_38_49 | 0.96 |
| Corticosteroids | LC-ECA | 13_78_992 | 0.96 |
| Antidepressant | LC-ECA | 13_92_333 | 0.96 |
| Anxioytic | LC-ECA | 4HBAC | 0.96 |
| Antidepressant | GC-TOF | 199553 | 0.97 |
| Antipsychotic | GC-TOF | 2_MONOSTEARIN_NIST | 0.97 |
| Corticosteroids | GC-TOF | 204318 | 0.97 |
| Corticosteroids | GC-TOF | 204994 | 0.97 |
| Statins | GC-TOF | 213227 | 0.97 |
| Statins | GC-TOF | 218597 | 0.97 |
| Statins | GC-TOF | 267675 | 0.97 |
| Statins | GC-TOF | 293097 | 0.97 |
| Statins | GC-TOF | 300379 | 0.97 |
| Statins | GC-TOF | 309788 | 0.97 |
| Statins | GC-TOF | LEUCINE | 0.97 |
| Antidepressant | GC-TOF | ORNITHINE | 0.97 |
| Anxioytic | GC-TOF | XYLULOSE_NIST | 0.97 |
| Corticosteroids | LC-ECA | KYN | 0.97 |
| Antipsychotic | LC-ECA | 13_86_8 | 0.97 |
| Statins | LC-ECA | 5_15_692 | 0.97 |
| Antidepressant | LC-ECA | 5_40_292 | 0.97 |
| Statins | GC-TOF | 199203 | 0.98 |
| Anxioytic | GC-TOF | 1_MONOSTEARIN | 0.98 |
| Antidepressant | GC-TOF | 202573 | 0.98 |
| Corticosteroids | GC-TOF | 208686 | 0.98 |
| Antidepressant | GC-TOF | 227655 | 0.98 |
| Anxioytic | GC-TOF | 234580 | 0.98 |
| Statins | GC-TOF | 239954 | 0.98 |
| Antidepressant | GC-TOF | 267650 | 0.98 |
| Statins | GC-TOF | 267650 | 0.98 |
| Antipsychotic | GC-TOF | 310831 | 0.98 |
| Corticosteroids | GC-TOF | 312902 | 0.98 |
| Statins | GC-TOF | ALANINE | 0.98 |
| Antidepressant | GC-TOF | ARABINOSE | 0.98 |
| Antidepressant | GC-TOF | ASCORBIC_ACID | 0.98 |
| Antipsychotic | GC-TOF | ASCORBIC_ACID | 0.98 |
| Antipsychotic | GC-TOF | CHOLESTEROL_{—} | 0.98 |
| Antidepressant | GC-TOF | CONDURITOL_BETA_EPDXIDE | 0.98 |
| Statins | GC-TOF | INOSITOL_MYO_{—} | 0.98 |
| Corticosteroids | GC-TOF | TAURINE | 0.98 |
| Statins | GC-TOF | XANTHINE | 0.98 |
| Statins | GC-TOF | 218767 | 0.99 |
| Statins | GC-TOF | 231674 | 0.99 |
| Statins | GC-TOF | 233340 | 0.99 |
| Antidepressant | GC-TOF | 234622 | 0.99 |
| Antidepressant | GC-TOF | GLYCOLIC_ACID | 0.99 |
| Corticosteroids | GC-TOF | INULOBIOSE_2 | 0.99 |
| Anxioytic | GC-TOF | RIBITOL | 0.99 |
| Antipsychotic | GC-TOF | SORBITOL | 0.99 |
| Corticosteroids | LC-ECA | 5HTP | 0.99 |
| Anxioytic | GC-TOF | TRYPTOPHAN | 1 |
| Anxioytic | LC-ECA | 11_60_917 | 1 |
| Corticosteroids | LC-ECA | 14_36_45 | 1 |

A list of the association results for each metabolite and ApoE genotype status are listed in Table 12. These metabolites were removed from the list of potential predictors for the next stage of analysis, leaving a total of 238 metabolites evaluated in the model building step.

TABLE 12
Metabolites' Association with ApoE Genotype Status

| | | K-Wallis |
|---|---|---|
| Platform | Metabolite | P-Value |
| GC-TOF | PHOSPHOETHANOLAMINE | 0.0053 |
| GC-TOF | 2_MONOPALMITIN | 0.0097 |
| GC-TOF | 218767 | 0.017 |
| GC-TOF | 202885 | 0.018 |
| GC-TOF | 2_MONOSTEARIN_NIST | 0.02 |
| GC-TOF | GLYCOLIC_ACID | 0.023 |
| GC-TOF | 280573 | 0.024 |
| GC-TOF | 281216 | 0.026 |
| GC-TOF | 293849 | 0.034 |
| GC-TOF | ACONITIC_ACID | 0.034 |
| GC-TOF | 204994 | 0.035 |
| GC-TOF | 201887 | 0.036 |
| GC-TOF | 227270 | 0.036 |
| GC-TOF | MALTOSE_1 | 0.038 |
| GC-TOF | 218787 | 0.043 |
| GC-TOF | 215494 | 0.047 |
| GC-TOF | 273984 | 0.047 |
| GC-TOF | 224035 | 0.048 |
| GC-TOF | 267884 | 0.048 |
| GC-TOF | 268306 | 0.049 |
| GC-TOF | 289055 | 0.05 |
| GC-TOF | 288019 | 0.051 |
| LC-ECA | 9_25_825 | 0.059 |
| GC-TOF | 234580 | 0.061 |
| GC-TOF | 268321 | 0.061 |
| GC-TOF | ASPARAGINE_DEH_{—} | 0.061 |
| GC-TOF | 199463 | 0.062 |
| GC-TOF | MONOPALMITIN_1_GLYCERIDE | 0.062 |
| GC-TOF | 1_MONOSTEARIN | 0.065 |
| GC-TOF | 269625 | 0.065 |
| GC-TOF | 312308 | 0.065 |
| GC-TOF | SUCCINIC_ACID | 0.068 |
| GC-TOF | 241920 | 0.074 |
| GC-TOF | 223830 | 0.077 |
| GC-TOF | 221597 | 0.083 |
| GC-TOF | 228885 | 0.086 |
| GC-TOF | 224020 | 0.087 |
| GC-TOF | PANTOTHENIC_ACID | 0.088 |
| GC-TOF | MANNITOL | 0.094 |
| GC-TOF | 204344 | 0.095 |
| GC-TOF | GLUCOHEPTULOSE | 0.096 |
| GC-TOF | 2_DEOXYRIBONIC_ACID | 0.097 |
| GC-TOF | CHOLESTEROL_{—} | 0.097 |
| GC-TOF | INULOBIOSE_2 | 0.098 |
| GC-TOF | 224551 | 0.1 |
| GC-TOF | 231657 | 0.1 |
| GC-TOF | 225548 | 0.11 |
| GC-TOF | 288808 | 0.11 |
| GC-TOF | 312902 | 0.11 |
| GC-TOF | N_ACETYL_D_MANNOSAMINE | 0.11 |
| GC-TOF | 239954 | 0.12 |
| GC-TOF | 267649 | 0.12 |
| LC-ECA | 8_28_508 | 0.12 |
| GC-TOF | 226303 | 0.13 |
| GC-TOF | 4_HYDROXYBUTYRIC_ACID | 0.13 |
| GC-TOF | GLYCERIC_ACID | 0.14 |
| GC-TOF | 281907 | 0.15 |
| GC-TOF | INOSINE | 0.15 |
| GC-TOF | 2_DEOXYTETRONIC_ACID | 0.16 |
| GC-TOF | 218597 | 0.16 |
| GC-TOF | 218951 | 0.16 |
| GC-TOF | 231056 | 0.16 |
| GC-TOF | 231326 | 0.16 |
| GC-TOF | 3_HYDROXYPROPIONIC_ACID | 0.16 |
| GC-TOF | THREITOL— | 0.16 |
| GC-TOF | THREONIC_ACID | 0.16 |
| LC-ECA | 12_94_5 | 0.16 |
| GC-TOF | 202573 | 0.17 |
| GC-TOF | 225863 | 0.17 |
| LC-ECA | 11_46_55 | 0.17 |
| LC-ECA | 12_52_75 | 0.17 |
| GC-TOF | 199777 | 0.18 |
| GC-TOF | 2_HYDROXYVALERIC_ACID | 0.18 |
| GC-TOF | 234015 | 0.18 |
| GC-TOF | 280940 | 0.18 |
| GC-TOF | 309788 | 0.18 |
| GC-TOF | BETA_MANNOSYLGLYCERATE_MINOR_{—} | 0.18 |
| GC-TOF | HEXURONIC_ACID | 0.18 |
| LC-ECA | 5_102_808 | 0.18 |
| GC-TOF | 228369 | 0.19 |
| GC-TOF | CONDURITOL_BETA_EPOXIDE | 0.19 |
| LC-ECA | 8_76_933 | 0.19 |
| GC-TOF | ASCORBIC_ACID | 0.2 |
| GC-TOF | ERYTHROSE | 0.2 |
| GC-TOF | THREONINE | 0.2 |
| LC-ECA | 15_68_542 | 0.2 |
| LC-ECA | 4HPLA | 0.2 |
| LC-ECA | 5_15_692 | 0.2 |
| LC-ECA | 8_89_433 | 0.2 |
| GC-TOF | 218710 | 0.21 |
| GC-TOF | 240432 | 0.21 |
| GC-TOF | 269160 | 0.21 |
| GC-TOF | 293848 | 0.21 |
| GC-TOF | 306152 | 0.21 |
| GC-TOF | BENZOIC_ACID | 0.21 |
| LC-ECA | 15_90_6 | 0.21 |
| GC-TOF | 213227 | 0.22 |
| GC-TOF | 229164 | 0.22 |
| GC-TOF | 231674 | 0.22 |
| GC-TOF | 294129 | 0.22 |
| GC-TOF | 301825 | 0.22 |
| GC-TOF | 200556 | 0.23 |
| LC-ECA | 8_63_675 | 0.23 |
| LC-ECA | 8_93_65 | 0.23 |
| GC-TOF | 234622 | 0.24 |
| GC-TOF | 309532 | 0.24 |
| GC-TOF | 310448 | 0.24 |
| GC-TOF | CYSTEINE | 0.24 |
| LC-ECA | ASA | 0.24 |
| GC-TOF | 231947 | 0.25 |
| GC-TOF | 238467 | 0.25 |
| GC-TOF | GLUCONIC_ACID | 0.25 |
| GC-TOF | 309642 | 0.26 |
| GC-TOF | TRYPTOPHAN | 0.26 |
| LC-ECA | VMA | 0.26 |
| LC-ECA | 14_22_758 | 0.26 |
| GC-TOF | 200906 | 0.27 |
| GC-TOF | 202599 | 0.27 |
| GC-TOF | 228528 | 0.27 |
| GC-TOF | 268579 | 0.27 |
| LC-ECA | HVA | 0.27 |
| GC-TOF | 217783 | 0.28 |
| GC-TOF | 228911 | 0.28 |
| GC-TOF | 312289 | 0.28 |
| GC-TOF | 312448 | 0.28 |
| GC-TOF | GLYCEROL_3_GALACTOSIDE | 0.28 |
| LC-ECA | URIC | 0.28 |
| LC-ECA | 12_50_183 | 0.28 |
| GC-TOF | 213697 | 0.29 |
| GC-TOF | GLUCOHEPTOSE | 0.29 |
| GC-TOF | RIBOSE | 0.29 |
| GC-TOF | 2_DEOXYTETRONIC_ACID_NIST | 0.3 |
| GC-TOF | 208686 | 0.31 |
| GC-TOF | 231099 | 0.31 |
| GC-TOF | 267650 | 0.31 |
| GC-TOF | CITRAMALATE | 0.31 |
| GC-TOF | 218513 | 0.32 |
| GC-TOF | LYSINE | 0.32 |
| GC-TOF | 227582 | 0.33 |
| GC-TOF | SALICYLALDEHYDE | 0.33 |
| GC-TOF | XYLULOSE_NIST | 0.33 |
| LC-ECA | MET | 0.33 |
| GC-TOF | 217870 | 0.34 |
| GC-TOF | 227655 | 0.34 |
| GC-TOF | 299416 | 0.34 |
| GC-TOF | DIHYDROXYMALONIC_ACID_NIST | 0.34 |
| GC-TOF | 309873 | 0.35 |
| GC-TOF | PALMITIC_ACID | 0.35 |
| LC-ECA | 13_78_992 | 0.35 |
| LC-ECA | 9_29_925 | 0.35 |
| GC-TOF | 3_HYDROXY_3_METHYL- | 0.36 |
| | GLUTARIC_ACID | |
| GC-TOF | 306159 | 0.36 |
| GC-TOF | ISOTHREONIC_ACID | 0.36 |
| LC-ECA | 2HPAC | 0.36 |
| GC-TOF | 223535 | 0.37 |
| GC-TOF | 224322 | 0.37 |
| GC-TOF | 214426 | 0.38 |
| LC-ECA | TRP | 0.38 |
| GC-TOF | ENOLPYRUVATE_NIST | 0.39 |
| GC-TOF | GLYCINE | 0.39 |
| GC-TOF | ISOLEUCINE | 0.39 |
| LC-ECA | 15_65_533 | 0.39 |
| LC-ECA | XANTH | 0.39 |
| GC-TOF | 236890 | 0.4 |
| GC-TOF | 310006 | 0.4 |
| GC-TOF | PELARGONIC_ACID | 0.4 |
| LC-ECA | GR | 0.4 |
| GC-TOF | 312362 | 0.41 |
| LC-ECA | 12_41_200 | 0.41 |
| LC-ECA | 13_18_475 | 0.41 |
| LC-ECA | 4_22_117 | 0.41 |
| GC-TOF | 200490 | 0.42 |
| LC-ECA | 4_32_592 | 0.42 |
| LC-ECA | 5_24_483 | 0.42 |
| GC-TOF | ASPARTIC_ACID | 0.43 |
| GC-TOF | 208755 | 0.44 |
| GC-TOF | 231097 | 0.44 |
| GC-TOF | ACETOPHENONE_NIST | 0.44 |
| GC-TOF | GLYCEROL_ALPHA_PHOSPHATE | 0.44 |
| LC-ECA | I3AA | 0.44 |
| LC-ECA | 13_84_975 | 0.44 |
| GC-TOF | 271416 | 0.45 |
| GC-TOF | TAURINE | 0.45 |
| GC-TOF | XANTHINE | 0.45 |
| GC-TOF | 309641 | 0.46 |
| GC-TOF | 211935 | 0.47 |
| GC-TOF | STEARIC_ACID | 0.47 |
| GC-TOF | 206308 | 0.48 |
| GC-TOF | PHOSPHATE | 0.48 |
| GC-TOF | SORBITOL | 0.48 |
| GC-TOF | 219683 | 0.49 |
| GC-TOF | 303060 | 0.49 |
| GC-TOF | 312645 | 0.49 |
| GC-TOF | 227387 | 0.5 |
| GC-TOF | INOSITOL_MYO_{—} | 0.51 |
| GC-TOF | LACTIC_ACID | 0.51 |
| LC-ECA | 13_54_95 | 0.51 |
| LC-ECA | 8_14_983 | 0.51 |
| GC-TOF | 227597 | 0.52 |
| GC-TOF | OXOPROLINE | 0.52 |
| GC-TOF | PUTRESCINE | 0.52 |
| LC-ECA | 13_38_49 | 0.52 |
| GC-TOF | 202572 | 0.53 |
| GC-TOF | 267665 | 0.53 |
| GC-TOF | 300379 | 0.53 |
| GC-TOF | 213198 | 0.54 |
| GC-TOF | 267737 | 0.54 |
| GC-TOF | TYROSINE | 0.54 |
| GC-TOF | 212208 | 0.55 |
| GC-TOF | 235414 | 0.55 |
| GC-TOF | 267816 | 0.55 |
| GC-TOF | 268483 | 0.55 |
| LC-ECA | 11_60_917 | 0.55 |
| GC-TOF | 204318 | 0.56 |
| GC-TOF | 224849 | 0.56 |
| GC-TOF | 310010 | 0.56 |
| GC-TOF | 215739 | 0.57 |
| GC-TOF | 270407 | 0.57 |
| GC-TOF | CREATININE | 0.58 |
| GC-TOF | FUCOSE | 0.58 |
| GC-TOF | LEUCINE | 0.58 |
| LC-ECA | PXAN | 0.58 |
| GC-TOF | PHENYLALANINE | 0.59 |
| LC-ECA | 5HTP | 0.59 |
| GC-TOF | 227652 | 0.6 |
| GC-TOF | 306156 | 0.6 |
| LC-ECA | MHPG | 0.6 |
| GC-TOF | 268313 | 0.61 |
| GC-TOF | 268365 | 0.61 |
| GC-TOF | 307965 | 0.61 |
| GC-TOF | 309934 | 0.61 |
| GC-TOF | ALANINE | 0.61 |
| GC-TOF | DEHYDROASCORBATE | 0.61 |
| GC-TOF | ERYTHRONIC_ACID_LACTONE | 0.61 |
| GC-TOF | FUCOSE_RHAMNOSE | 0.61 |
| LC-ECA | 14_64_275 | 0.61 |
| GC-TOF | 2_HYDROXYBUTANOIC_ACID | 0.62 |
| GC-TOF | 231709 | 0.62 |
| GC-TOF | 238149 | 0.62 |
| GC-TOF | 312622 | 0.62 |
| GC-TOF | XYLITOL | 0.62 |
| LC-ECA | 11_51_158 | 0.62 |
| GC-TOF | 203235 | 0.63 |
| GC-TOF | 268709 | 0.63 |
| GC-TOF | 312592 | 0.63 |
| LC-ECA | 13_74_392 | 0.63 |
| GC-TOF | 208655 | 0.64 |
| GC-TOF | 224635 | 0.64 |
| GC-TOF | 232604 | 0.64 |
| GC-TOF | 242417 | 0.64 |
| GC-TOF | 308328 | 0.64 |
| GC-TOF | 293097 | 0.65 |
| GC-TOF | SERINE | 0.65 |
| LC-ECA | 14_36_45 | 0.65 |
| GC-TOF | 216428 | 0.66 |
| GC-TOF | 267675 | 0.66 |
| GC-TOF | 306157 | 0.66 |
| GC-TOF | ETHANOLAMINE | 0.66 |
| GC-TOF | ORNITHINE | 0.66 |
| GC-TOF | PUTREANINE_NIST | 0.66 |
| GC-TOF | 222169 | 0.68 |
| GC-TOF | 231544 | 0.68 |
| GC-TOF | 231850 | 0.68 |
| GC-TOF | XYLOSE | 0.68 |
| LC-ECA | 11_36_75 | 0.68 |
| GC-TOF | 213193 | 0.69 |
| GC-TOF | ISOCITRIC_ACID | 0.69 |
| GC-TOF | VALINE | 0.69 |
| GC-TOF | 213182 | 0.7 |
| GC-TOF | ARABITOL | 0.7 |
| GC-TOF | PROLINE | 0.7 |
| GC-TOF | 212274 | 0.71 |
| GC-TOF | 215978 | 0.71 |
| GC-TOF | 228557 | 0.71 |
| GC-TOF | 241097 | 0.72 |
| GC-TOF | GLUTAMINE_DEH_{—} | 0.72 |
| LC-ECA | 15_77_017 | 0.72 |
| LC-ECA | 5HIAA | 0.72 |
| GC-TOF | PSEUDO_URIDINE | 0.73 |
| LC-ECA | 9_20_858 | 0.73 |
| GC-TOF | 238270 | 0.74 |
| GC-TOF | 268420 | 0.74 |
| LC-ECA | KYN | 0.74 |
| LC-ECA | 9_29_34 | 0.74 |
| GC-TOF | 2_KETOISOCAPROIC_ACID | 0.75 |
| GC-TOF | 204425 | 0.75 |
| GC-TOF | 309545 | 0.75 |
| GC-TOF | 301583 | 0.76 |
| GC-TOF | 312679 | 0.76 |
| GC-TOF | 2_DEOXYERYTHRITOL | 0.77 |
| GC-TOF | GLYCEROL | 0.77 |
| GC-TOF | SUCROSE | 0.77 |
| GC-TOF | 210286 | 0.78 |
| GC-TOF | ALPHA_KETOGLUTARIC_ACID | 0.78 |
| GC-TOF | METHYLHEXADECANOIC_ACID | 0.78 |
| LC-ECA | 4HBAC | 0.78 |
| LC-ECA | 9_33_1 | 0.78 |
| GC-TOF | 202091 | 0.79 |
| GC-TOF | 309573 | 0.8 |
| GC-TOF | GLUCOSE | 0.8 |
| GC-TOF | 281257 | 0.82 |
| GC-TOF | IDONIC_ACID_NIST | 0.82 |
| LC-ECA | 13_86_8 | 0.82 |
| GC-TOF | 200541 | 0.83 |
| GC-TOF | 213143 | 0.83 |
| GC-TOF | 310831 | 0.83 |
| GC-TOF | 201005 | 0.84 |
| GC-TOF | N_METHYLALANINE | 0.84 |
| GC-TOF | THYMINE | 0.84 |
| LC-ECA | 13_44_608 | 0.84 |
| GC-TOF | 228605 | 0.85 |
| GC-TOF | 3_HYDROXYBUTANOIC_ACID | 0.85 |
| GC-TOF | 199203 | 0.86 |
| GC-TOF | 226853 | 0.86 |
| GC-TOF | CITRIC_ACID | 0.86 |
| GC-TOF | MYRISTIC_ACID | 0.86 |
| LC-ECA | I3PA | 0.86 |
| LC-ECA | 13_92_333 | 0.86 |
| LC-ECA | XAN | 0.86 |
| GC-TOF | 199806 | 0.87 |
| GC-TOF | 296108 | 0.87 |
| GC-TOF | FRUCTOSE | 0.87 |
| LC-ECA | HX | 0.87 |
| GC-TOF | UREA | 0.88 |
| LC-ECA | 14_75_608 | 0.88 |
| LC-ECA | 4HPAC | 0.88 |
| GC-TOF | 210168 | 0.89 |
| GC-TOF | GLUTAMIC_ACID | 0.89 |
| GC-TOF | 200595 | 0.9 |
| GC-TOF | PHOSPHORIC_ACID | 0.9 |
| GC-TOF | 199553 | 0.91 |
| GC-TOF | 226906 | 0.91 |
| GC-TOF | 226935 | 0.91 |
| GC-TOF | 280546 | 0.91 |
| GC-TOF | 203765 | 0.92 |
| GC-TOF | 301536 | 0.92 |
| GC-TOF | 308185 | 0.93 |
| LC-ECA | 5_40_292 | 0.93 |
| GC-TOF | 226851 | 0.94 |
| GC-TOF | 233340 | 0.94 |
| GC-TOF | 296106 | 0.94 |
| GC-TOF | 301325 | 0.94 |
| GC-TOF | ARABINOSE | 0.94 |
| GC-TOF | METHIONINE | 0.94 |
| LC-ECA | 5_25_075 | 0.94 |
| GC-TOF | 223505 | 0.95 |
| GC-TOF | 3_DEOXYPENTITOL_NIST | 0.95 |
| GC-TOF | 309538 | 0.95 |
| GC-TOF | RIBITOL | 0.95 |
| LC-ECA | GSH | 0.95 |
| GC-TOF | 228612 | 0.97 |
| GC-TOF | 232485 | 0.97 |
| GC-TOF | 233005 | 0.97 |
| GC-TOF | 241881 | 0.97 |
| GC-TOF | 270351 | 0.97 |
| GC-TOF | INOSITOL_ALLO_{—} | 0.97 |
| LC-ECA | TYR | 0.97 |
| GC-TOF | 228147 | 0.99 |
| GC-TOF | ERYTHRITOL | 0.99 |
| LC-ECA | 14_34_25 | 0.99 |
| GC-TOF | 307889 | 1 |
| GC-TOF | 312977 | 1 |
| LC-ECA | 13_19_492 | 1 |
| LC-ECA | 8_82_917 | 1 |
| LC-ECA | 9_19_067 | 1 |

### Example 8

### Model Building

The summary of the model fits from the stepwise logistic regression modeling for the AD vs. control is listed in Table 13.

TABLE 13
Summary Measures of Model Fit for Each Resulting Model for
Discriminating between AD vs. Controls in the Full Dataset

| | | | Model | | | |
|---|---|---|---|---|---|---|
| Model Variables | | | Variable | | | |
| E | M | P | Abbreviations | AUC | Sensitivity | Specificity |
| X | | | E | 0.96 | 1.00* | 0.90 |
| | X | | M | 0.70 | 0.68 | 0.67 |
| | | X | P | 0.92 | 0.97 | 0.83 |
| X | | X | P\|E | 0.90 | 0.93 | 0.89 |
| | X | X | P\|M | 0.89 | 0.88 | 0.88 |
| X | X | X | P\|M\|E | 0.90* | 0.92 | 0.89 |

AD, Alzheimer's Disease; AUC, Area under the Curve;
*rounded.

The final models are listed Table 14, with the logistic regression equation (with parameter estimates and included variables listed) for each cross-validation interval.

TABLE 14
Final Logistic Regression Models for the AD vs. control

| Variable | | | | | | |
|---|---|---|---|---|---|---|
| Family | | | | | | |
| Included In | Fold | | AUC | R-Squared | AUC | R-Squared |
| Modeling | (CV) | Equation | (Train) | (Train) | (Test) | (Test) |
| P | 1 | logit(Case_control) = 3.99*(Intercept) + 0.0126*Ttau − 0.0262*Abeta42 | 0.92 | 0.64 | 0.93 | NA |
| P | 2 | logit(Case_control) = 7.05*(Intercept) − 0.037*Abeta42 | 0.93 | 0.67 | 0.85 | 0.27 |
| P | 3 | logit(Case_control) = 6.53*(Intercept) − 0.0378*Abeta42 | 0.93 | 0.69 | 0.91 | 0.57 |
| P | 4 | logit(Case_control) = 6.53*(Intercept) − 0.0358*Abeta42 | 0.92 | 0.64 | 0.89 | 0.58 |
| P | 5 | logit(Case_control) = 5.71*(Intercept) − 0.031*Abeta42 | 0.91 | 0.61 | 1.00 | 1.00 |
| A\|P | 1 | logit(Case_control) = 4.51*(Intercept) + 0.0217*Ptau − 0.0283*Abeta42 | 0.92 | 0.64 | 0.90 | NA |
| A\|P | 2 | logit(Case_control) = 5.41*(Intercept) − 0.0292*Abeta42 | 0.91 | 0.60 | 0.98 | 0.91 |
| A\|P | 3 | logit(Case_control) = 4.71*(Intercept) + 0.0136*Ttau − 0.0314*Abeta42 | 0.94 | 0.73 | 0.83 | 0.45 |
| A\|P | 4 | logit(Case_control) = 7.41*(Intercept) − 0.0404*Abeta42 | 0.92 | 0.67 | 0.94 | 0.52 |
| A\|P | 5 | logit(Case_control) = 6.22*(Intercept) − 0.0339*Abeta42 | 0.91 | 0.64 | 0.89 | 0.66 |
| P\|A\|M | 1 | logit(Case_control) = −77.3*(Intercept) + 1.77*ApoE.index − | 0.97 | 0.84 | 0.91 | 0.84 |
| | | 0.0606*Abeta42 + 16.5*M_199777 | | | | |
| P\|A\|M | 2 | logit(Case_control) = −42.4*(Intercept) − 0.0471*Abeta42 + 10.3*M_199777 − | 0.97 | 0.78 | 1.00 | 1.00 |
| | | 1.36*M_222169 | | | | |
| P\|A\|M | 3 | logit(Case_control) = −15*(Intercept) − | 0.97 | 0.81 | 0.84 | 1.00 |
| | | 0.0497*Abeta42 + 3.19*M_268306 + 5.85*M_307889 − 2.29*M_222169 | | | | |
| P\|A\|M | 4 | logit(Case_control) = −17500*(Intercept) − 183*ApoE.index − 1.04*Ttau − | 1.00 | NA | 0.86 | 1.00 |
| | | 46.2*Abeta42 + 2700*M_268306 + 2000*M_199777 + 3580*M_Gluconic_Acid − | | | | |
| | | 1000*M_307889 − 1910*M_222169 | | | | |
| P\|A\|M | 5 | logit(Case_control) = −41.9*(Intercept) + 0.0212*Ttau − | 0.97 | 0.83 | 0.90 | 1.00 |
| | | 0.0411*Abeta42 + 9.77*M_199777 − 1.39*M_222169 | | | | |
| P\|M | 1 | logit(Case_control) = −36.1*(Intercept) − | 0.96 | 0.80 | 0.95 | 1.00 |
| | | 0.0474*Abeta42 + 12.5*M_Gluconic—Acid − 1.78*M_222169 | | | | |
| P\|M | 2 | logit(Case_control) = −44.6*(Intercept) − | 0.97 | 0.82 | 0.92 | 0.87 |
| | | 0.0501*Abeta42 + 5.79*M_268306 + 7.38*M_199777 − 2.46*M_222169 | | | | |
| P\|M | 3 | logit(Case_control) = −52.6*(Intercept) − | 0.98 | 0.87 | 0.90 | 1.00 |
| | | 0.0753*Abeta42 + 4.24*M_268306 + 11.5*M_199777 − 3.82*M_222169 | | | | |
| P\|M | 4 | logit(Case_control) = −4300*(Intercept) − 2.26*Ttau − | 1.00 | NA | 0.69 | 1.00 |
| | | 19.8*Abeta42 + 1690*M_Gluconic_Acid + 549*M_307889 − 136*M_222169 | | | | |
| P\|M | 5 | logit(Case_control) = −43.8*(Intercept) − | 0.97 | 0.80 | 1.00 | 1.00 |
| | | 0.0487*Abeta42 + 2.43*M_268306 + 9.22*M_199777 − 1.88*M_222169 | | | | |
| M | 1 | logit(Case_control) = −23.4*(Intercept) + 0.551*M_268306 + 4.39*M_199777 − | 0.74 | 0.24 | 0.84 | 0.71 |
| | | 0.541*M_222169 | | | | |
| M | 2 | logit(Case_control) = −21.4*(Intercept) + 0.596*M_268306 + 4*M_199777 − | 0.75 | 0.26 | 0.75 | 0.29 |
| | | 0.736*M_222169 | | | | |
| M | 3 | logit(Case_control) = −13.4*(Intercept) + 4.22*M_Gluconic_Acid − | 0.76 | 0.29 | 0.49 | 0.15 |
| | | 1.16*M_222169 | | | | |
| M | 4 | logit(Case_control) = −21.3*(Intercept) + 5.89*M_Gluconic_Acid − | 0.75 | 0.27 | 0.65 | 0.16 |
| | | 0.682*M_222169 | | | | |
| M | 5 | logit(Case_control) = −27*(Intercept) + 4.07*M_268306 + | 0.79 | 0.34 | 0.77 | 0.31 |
| | | 3.59*M_Gluconic_Acid − 0.87*M_222169 | | | | |
| A\|M | 1 | logit(Case_control) = −22.8*(Intercept) + 1.34*ApoE.index + 4.63*M_199777 − | 0.78 | 0.29 | 0.79 | 0.62 |
| | | 0.809*M_222169 | | | | |
| A\|M | 2 | logit(Case_control) = −37.6*(Intercept) + 1.81*ApoE.index + 7.6*M_199777 − | 0.82 | 0.43 | 0.72 | 0.27 |
| | | 1.36*M_222169 | | | | |
| A\|M | 3 | logit(Case_control) = −12.2*(Intercept) + 1.32*ApoE.index + | 0.77 | 0.29 | 0.73 | 0.47 |
| | | 3.52*M_Gluconic_Acid − 0.754*M_222169 | | | | |
| A\|M | 4 | logit(Case_control) = −24.9*(Intercept) + 0.873*ApoE.index + | 0.83 | 0.42 | 0.75 | 0.43 |
| | | 2.81*M_268306 + 4.29*M_Gluconic_Acid − 0.968*M_222169 | | | | |
| A\|M | 5 | logit(Case_control) = −31.6*(Intercept) + 0.96*ApoE.index + 5.88*M_199777 | 0.74 | 0.26 | 0.64 | 0.31 |
| E | 1 | logit(Case_control) = −898*(Intercept) + 3.66*E_15_65_533 + 11.8*E_12_94_5 − | 1.00 | NA | 0.93 | 1.00 |
| | | 6.53*E_8_93_65 + 2.69*E_8_89_433 | | | | |
| E | 2 | logit(Case_control) = −24*(Intercept) + 0.599*E_15_65_533 + | 0.99 | 0.92 | 0.93 | 0.75 |
| | | 0.0402*E_12_94_5 − 0.0907*E_14_64_275 | | | | |
| E | 3 | logit(Case_control) = −15.3*(Intercept) + 0.408*E_15_65_533 + | 0.98 | 0.87 | 1.00 | 1.00 |
| | | 0.0738*E_12_94_5 − 0.0859*E_8_93_65 | | | | |
| E | 4 | logit(Case_control) = −15.3*(Intercept) + 0.406*E_15_65_533 + | 0.98 | 0.86 | 1.00 | 1.00 |
| | | 0.0715*E_12_94_5 − 0.0821*E_8_93_65 | | | | |
| E | 5 | logit(Case_control) = −120*(Intercept) + 10.8*E_15_65_533 + | 1.00 | NA | 0.92 | 1.00 |
| | | 4.56*E_12_94_5 − 8.11*E_8_93_65 | | | | |
| P\|E | 1 | logit(Case_control) = −1390*(Intercept) + 38.6*E_15_65_533 | 1.00 | NA | 0.81 | 0.68 |
| P\|E | 2 | logit(Case_control) = −17*(Intercept) + 0.0143*Ttau + | 0.98 | 0.87 | 1.00 | 1.00 |
| | | 0.405*E_15_65_533 + 0.0785*E_12_94_5 − 0.0868*E_8_93_65 | | | | |
| P\|E | 3 | logit(Case_control) = −19.6*(Intercept) + 0.0567*Ptau + | 0.99 | 0.90 | 1.00 | 1.00 |
| | | 0.359*E_15_65_533 + 0.0353*E_12_94_5 | | | | |
| P\|E | 4 | logit(Case_control) = −568*(Intercept) + 1.01*Ptau + 5.79*E_12_94_5 | 1.00 | 1.00 | 0.69 | 0.09 |
| P\|E | 5 | logit(Case_control) = −14.8*(Intercept) + 0.403*E_15_65_533 + | 0.98 | 0.89 | 1.00 | 1.00 |
| | | 0.0801*E_12_94_5 − 0.101*E_8_93_65 | | | | |
| A\|E | 1 | logit(Case_control) = −14.5*(Intercept) − 0.397*E_15_65_533 + | 0.97 | 0.85 | 1.00 | 1.00 |
| | | 0.0743*E_12_94_5 − 0.0896*E_8_93_65 | | | | |
| A\|E | 2 | logit(Case_control) = −1330*(Intercept) + 8.14*E_15_65_533 + | 1.00 | NA | 0.90 | 1.00 |
| | | 13.7*E_12_94_5 − 7.47*E_8_93_65 + 4.44*E_8_89_433 | | | | |
| A\|E | 3 | logit(Case_control) = −18.3*(Intercept) + 0.388*E_15_65_533 + | 0.98 | 0.86 | 0.98 | NA |
| | | 0.0331*E_12_94_5 | | | | |
| A\|E | 4 | logit(Case_control) = −2570*(Intercept) + 69.2*E_15_65_533 + | 1.00 | NA | 0.94 | 1.00 |
| | | 20.4*E_12_94_5 − 27.9*E_8_93_65 + 14.5*E_14_64_275 | | | | |
| A\|E | 5 | logit(Case_control) = −15*(Intercept) + 0.398*E_15_65_533 + | 0.98 | 0.87 | 1.00 | 1.00 |
| | | 0.0673*E_12_94_5 − 0.0778*E_8_93_65 | | | | |
| A\|P\|E | 1 | logit(Case_control) = −8.9*(Intercept) − | 0.98 | 0.88 | 1.00 | 1.00 |
| | | 0.0299*Abeta42 + 0.279*E_15_65_533 + 0.0336*E_12_94_5 | | | | |
| A\|P\|E | 2 | logit(Case_control) = −2550*(Intercept) + 9.49*Ptau + | 1.00 | NA | 0.93 | 1.00 |
| | | 13.3*E_15_65_533 + 20.3*E_12_94_5 − 1.92*E_14_64_275 | | | | |
| A\|P\|E | 3 | logit(Case_control) = −2150*(Intercept) + 44.7*E_15_65—533 + | 1.00 | NA | 0.85 | 0.69 |
| | | 4.16*E_12_94_5 | | | | |
| A\|P\|E | 4 | logit(Case_control) = −13500*(Intercept) + 10*Ttau − | 1.00 | NA | 0.77 | 1.00 |
| | | 11.1*Abeta42 + 590*E_15_65_533 − 165*E_14_64_275 | | | | |
| A\|P\|E | 5 | logit(Case_control) = −20.8*(Intercept) + 0.0341*Ttau + | 0.99 | 0.90 | 0.92 | 1.00 |
| | | 0.36*E_15_65_533 + 0.0382*E_12_94_5 | | | | |
| A\|M\|E | 1 | logit(Case_control) = −1160*(Intercept) + 23.4*E_15_65_533 + | 1.00 | NA | 0.89 | 0.76 |
| | | 3.56*E_8_89_433 | | | | |
| A\|M\|E | 2 | logit(Case_control) = −10600*(Intercept) − 1140*ApoE.index + | 1.00 | NA | 0.84 | 1.00 |
| | | 1700*M_268306 − 825*M_222169 + 172*E_15_65_533 + 9.47*E_8_89_433 − | | | | |
| | | 6.51*E_14_64_275 | | | | |
| A\|M\|E | 3 | logit(Case_control) = −1470*(Intercept) − 311*ApoE.index + 249*M_307889 − | 1.00 | NA | 0.81 | 1.00 |
| | | 164*M_222169 + 43*E_15_65_533 − 3.93*E_8_93_65 − 2.15*E_14_64_275 | | | | |
| A\|M\|E | 4 | logit(Case_control) = −2940*(Intercept) − | 1.00 | NA | 0.89 | 1.00 |
| | | 293*M_222169 + 106*E_15_65_533 + 4.65*E_8_89_433 − 15.9*E_14_64_275 | | | | |
| A\|M\|E | 5 | logit(Case_control) = −8680*(Intercept) + 343*M_268306 + | 1.00 | NA | 0.93 | 1.00 |
| | | 1220*M_Gluconic_Acid − 615*M_222169 + 95.4*E_15_65_533 + | | | | |
| | | 6.81*E_8_89_433 | | | | |
| P\|M\|E | 1 | logit(Case_control) = −1800*(Intercept) − 5.9*Abeta42 − 2910*M_199777 + | 1.00 | NA | 1.00 | 1.00 |
| | | 3740*M_Gluconic_Acid − 609*M_222169 + 85.8*E_15_65_533 + | | | | |
| | | 21.7*E_8_93_65 + 2.42*E_8_89_433 | | | | |
| P\|M\|E | 2 | logit(Case_control) = −923*(Intercept) − 3.06*Abeta42 + 565*M_268306 − | 1.00 | NA | 0.89 | 0.75 |
| | | 255*M_222169 + 2.1*E_14_64_275 | | | | |
| P\|M\|E | 3 | logit(Case_control) = −730*(Intercept) − | 1.00 | NA | 0.78 | 0.57 |
| | | 1.97*Abeta42 + 19.9*E_15_65_533 + 2.87*E_12_94_5 | | | | |
| P\|M\|E | 4 | logit(Case_control) = −15300*(Intercept) − 502*M_199777 + | 1.00 | NA | 0.90 | 1.00 |
| | | 4060*M_Gluconic_Acid − 628*M_307889 + 81.1*E_15_65_533 + | | | | |
| | | 8.2*E_12_94_5 − 5.29*E_14_64_275 | | | | |
| P\|M\|E | 5 | logit(Case_control) = −12700*(Intercept) − 12.6*Abeta42 − | 1.00 | NA | 0.94 | 1.00 |
| | | 1190*M_199777 + 4220*M_Gluconic_Acid − 544*M_222169 + | | | | |
| | | 116*E_15_65_533 + 26*E_8_93_65 − 14*E_14_64_275 | | | | |
| A\|P\|M\|E | 1 | logit(Case_control) = −1400*(Intercept) − 178*ApoE.index + 2.97*Ttau − | 1.00 | NA | 0.73 | 1.00 |
| | | 324*M_222169 + 54.9*E_15_65_533 | | | | |
| A\|P\|M\|E | 2 | logit(Case_control) = −563000*(Intercept) − 36200*ApoE.index − | 1.00 | NA | 0.88 | 1.00 |
| | | 161*Abeta42 + 137000*M_Gluconic_Acid − 32100*M_307889 + | | | | |
| | | 4780*E_15_65_533 − 525*E_14_64_275 | | | | |
| A\|P\|M\|E | 3 | logit(Case_control) = −50100*(Intercept) − 10.6*Abeta42 + | 1.00 | NA | 0.83 | 1.00 |
| | | 11100*M_268306 − 2180*M_222169 + 481*E_15_65_533 | | | | |
| A\|P\|M\|E | 4 | logit(Case_control) = −1780*(Intercept) − 3.41*Abeta42 + | 1.00 | NA | 0.89 | 1.00 |
| | | 281*M_307889 − 115*M_222169 − 12.9*E_12_94_5 + 33*E_8_93_65 + | | | | |
| | | 3.53*E_14_64_275 | | | | |
| A\|P\|M\|E | 5 | logit(Case_control) = −1890*(Intercept) − 1.49*Abeta42 + | 1.00 | NA | 0.87 | 0.59 |
| | | 445*M_Gluconic_Acid + 2.66*E_8_93_65 + 2.11*E_8_89_433 | | | | |

A, ApoE; P, Phospo; M, MassSpec Metabolites; E, Electrochem Metabolites

All models were statistically significant according to the results of the permutation testing (p<0.05 in all cases). The results of the Delong's test comparisons of the discrimination of the models are shown in Table 15, including the results for all two-way combinations of resulting models. Only the statistically significant p-values (using a Bonferroni correction) are listed.

TABLE 15
Results from the Comparisons of Prediction Models Built
on Luminex Phosphorylated Proteins (P), GC-TOF Mass Spectrometry
Metabolites (M) and LC-ECA Metabolites (E)

| | |
|---|---|
| Data | AUC Test |

| Types | E | M | P | P\|E | P\|M | P\|M\|E |
|---|---|---|---|---|---|---|
| E | | — | — | — | — | — |
| M | 0.005 | | — | — | — | — |
| P | | 0.004 | | — | — | — |
| P\|E | | 0.005 | | | — | — |
| P\|M | .045 | 0.005 | | | | — |
| P\|M\|E | | 0.004 | | | | |

AD, Alzheimer's Disease; AUC, Area under the Curve.

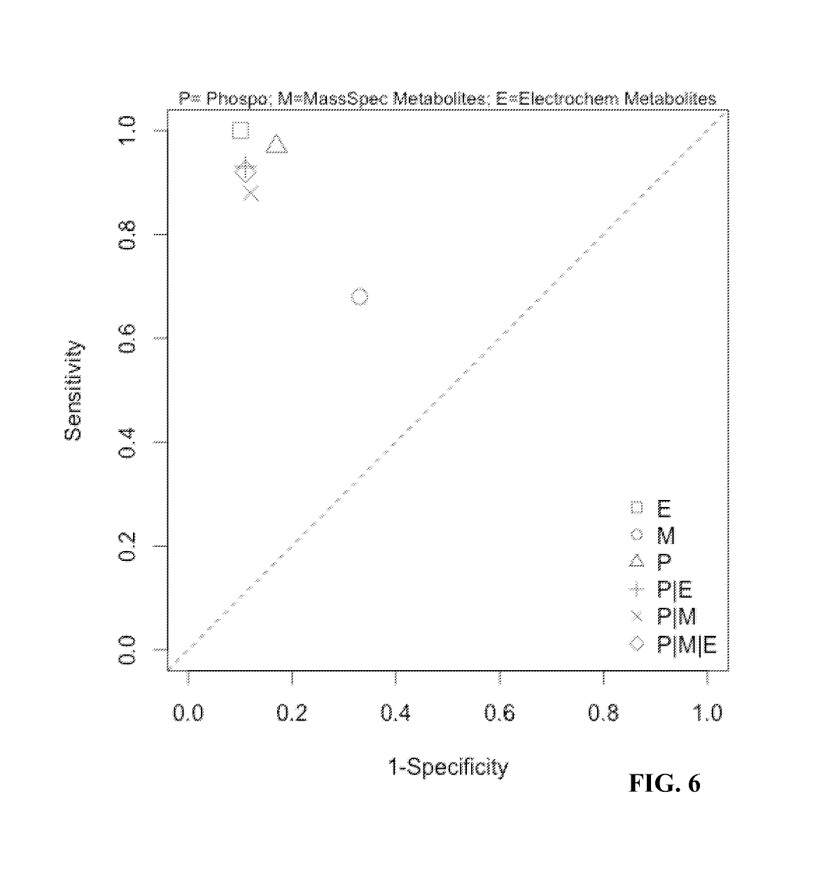

As expected, the model built with CSF Aβ, t-tau and p-tau levels as measured in Luminex immunoassays showed good discrimination of AD versus controls with an average testing AUC of 0.92 (Table 13). The model with the LC-ECA metabolites was also highly discriminatory with an average testing AUC of 0.96, slightly higher than the model built with the Luminex proteins. Remarkably, this discrimination was achieved with two metabolites that consistently were included in each cross-validation interval (5/5 cross-validation consistency): 15-65.533 and 8-93.65 the identities of which are currently unknown. By comparison, the GC-TOF mass spectrometry metabolites resulted in a model with much lower predictive performance (average testing AUC of 0.70) than the LC-ECA metabolites or Luminex proteins. Combining metabolomics with pathology markers did not increase accuracy much more and in some combinations reduced accuracy. To visually discriminate the predictive power of the models, the average performance (sensitivity and specificity) for each resulting model is depicted in FIG. 6. In this figure, better performance was seen as a lift in the models to the upper left quadrant of the graph.

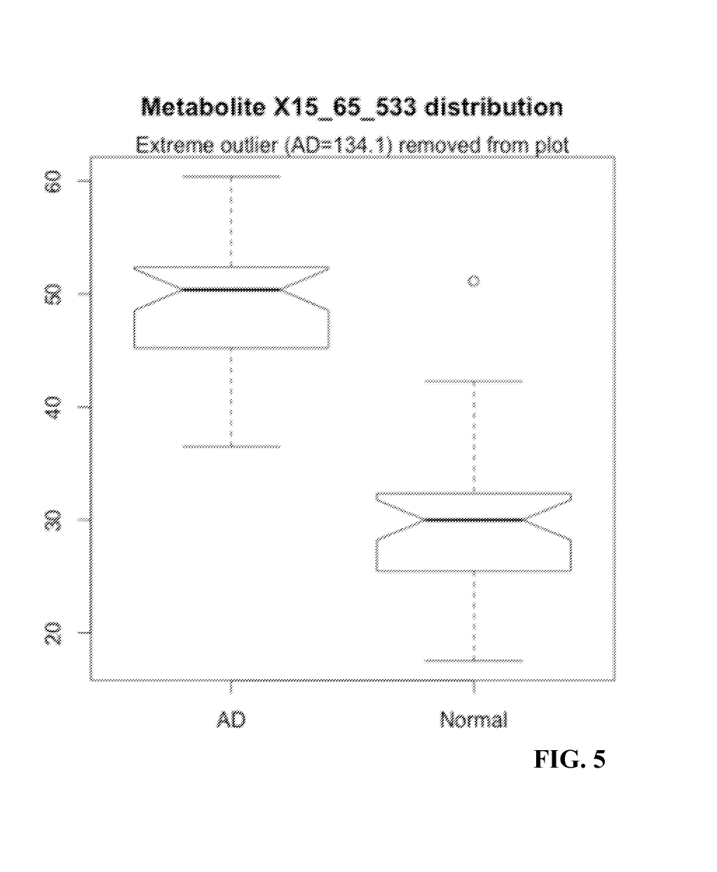

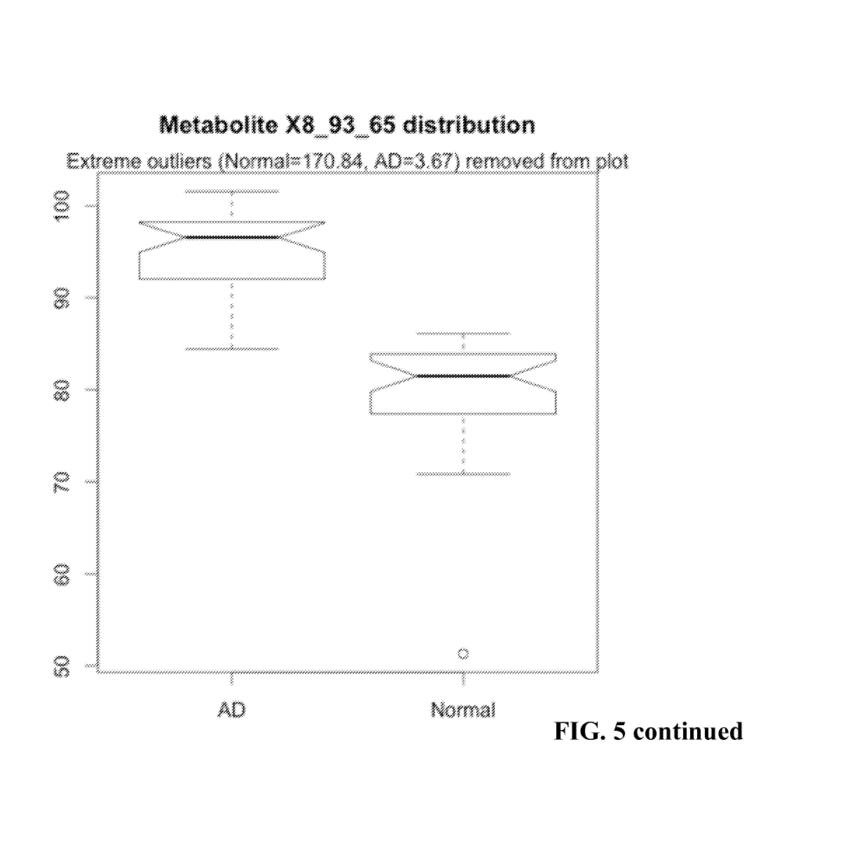

Since metabolites 15-65.533 and 8-93.65 had the most consistent association, FIG. 5 shows the distribution values of these metabolites for AD and control participants. It can be clearly seen that both of them are elevated in AD. This correlation analysis indicated that there was only weak correlation between these metabolites and the MMSE score (not statistically significant).

For the metabolites 8-93.65 and 15-65.533, associations with known metabolites were determined. These associations are shown in Table 16.

TABLE 16
Known Metabolites that are associated
with 8-93.65 and/or 15-65.533

| | Known | Correlation | | |
|---|---|---|---|---|
| | Metabolites | coefficient | P-value | Q-value |
| 8-93.65 | MET | 0.6 | 2.50E−12 | 1.10E−10 |
| | GSH | 0.38 | 0.000029 | 0.0002 |
| | 5-HIAA | 0.34 | 0.00025 | 0.0012 |
| | TYR | 0.32 | 0.00058 | 0.0024 |
| | TRP | 0.31 | 0.00089 | 0.0033 |
| | 4-HBAC | 0.29 | 0.0018 | 0.0057 |
| | VMA | 0.21 | 0.027 | 0.044 |
| 15-65.533 | I-3-PA | 0.54 | 5.40E−10 | 1.60E−08 |
| | MET | 0.44 | 0.000001 | 0.000012 |
| | KYN | 0.36 | 0.000067 | 0.00037 |
| | I-3-AA | 0.26 | 0.0047 | 0.013 |
| | GR | 0.24 | 0.0093 | 0.021 |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A method of diagnosing cognitive impairment in a subject in need thereof, the method comprising:
(a) obtaining a sample from the subject;
(b) measuring a level of one or more metabolites in the sample; and
(c) comparing the level measured in step (b) with a level of the one or more metabolites in a control,
wherein a change in the level of the one or more metabolites as compared to the control indicates that the subject is suffering from cognitive impairment, and
wherein the one or metabolites are in a pathway selected from the group consisting of a metabolic pathway, a tryptophan pathway, a tyrosine pathway, a purine pathway, a cysteine and methionine pathway, and any combination thereof.

2. The method of claim 1, wherein the sample is a cerebrospinal fluid sample.

3. The method of claim 1, wherein the cognitive impairment is selected from the group consisting of Alzheimer's Disease and mild cognitive impairment.

4. The method of claim 3, wherein the pathway is the tryptophan pathway and wherein the one or more metabolites is selected from the group consisting of tryptophan (TRP), 5-hydroxyindoleacetic acid (5-HIAA), 5-hydroxytryptophan (5-HTP), kynurenine (KYN), indole-3-acetic acid (I-3-AA), and any combination thereof.

5. The method of claim 4, wherein the one or more metabolites is 5-HIAA and wherein an increase in the level of 5-HIAA as compared to the control indicates that the subject is suffering from cognitive impairment.

6. The method of claim 4, wherein an increase in the level of I-3-AA, an increase in the level of KYN, or a decrease in the level of TRP as compared to the control indicates that the subject is suffering from mild cognitive impairment.

7. The method of claim 4, wherein the one or more metabolites are 5-HIAA and 5-HTP and wherein an increase in a ratio of the levels of 5-HIAA:5-HTP as compared to the control indicates that the subject is suffering from cognitive impairment.

8. The method of claim 4, wherein an increase in a ratio of the levels of KYN:TRP, an increase in a ratio of the levels of I-3-AA:TRP, or a decrease in a ratio of the levels of 5-HTP:TRP as compared to the control indicates that the subject is suffering from mild cognitive impairment.

9. The method of claim 4, further comprising
(d) comparing the level measured in step (b) with a level of the one or more metabolites in a subject suffering from mild cognitive impairment; and
(e) determining the subject is suffering from Alzheimer's disease when the level measured in step (b) is lower than the level of the one or more metabolites in the subject suffering from mild cognitive impairment,
wherein the one or more metabolites is 5-HTP.

10. The method of claim 3, wherein the pathway is the tyrosine pathway, wherein the one or more metabolites is vanillylmadelic acid (VMA), and wherein an increase in the level of VMA as compared to the control indicates that the subject is suffering from Alzheimer's Disease.

11. The method of claim 3, wherein the pathway is the purine pathway, wherein the one or more metabolites is xanthosine (XANTH), and wherein an increase in the level of XANTH as compared to the control indicates that the subject is suffering from Alzheimer's Disease.

12. The method of claim 3, wherein the pathway is the purine pathway, wherein the one or more metabolites is selected from the group consisting of hypoxanthine (HX) and uric acid (URIC), and wherein an increase in the level of HX or URIC as compared to the control indicates that the subject is suffering from mild cognitive impairment.

13. The method of claim 3, wherein the pathway is the purine pathway, wherein the one more metabolites is selected from the group consisting of uric acid (URIC), xanthine (XAN), xanthosine (XANTH), and hypoxanthine (HX), and wherein an increase in a ratio of the levels of URIC:XAN, an increase in a ratio of the levels of XAN:XANTH, or a decrease in a ratio of the levels of XAN:HX as compared to the control indicates that the subject is suffering from mild cognitive impairment.

14. The method of claim 3, wherein the pathway is the cysteine and methionine pathway, wherein the one or more metabolites is selected from the group consisting of methionine (MET) and glutathione (GSH), and wherein an increase in the level of MET or a decrease in a ratio of the levels of GSH:MET as compared to the control indicates that the subject is suffering from cognitive impairment.

15. The method of claim 3, wherein the one or more metabolites is selected from the group consisting of 15-65.533 and 8-93.65 and wherein an increase in the level of 15-65.533 or an increase in the level of 8-93.65 as compared to the control indicates that the subject is suffering from Alzheimer's Disease.

16. The method of claim 3, further comprising
(d) comparing the level measured in step (b) with a level of the one or more metabolites in a subject suffering from mild cognitive impairment; and
(e) determining the subject is suffering from Alzheimer's disease when the level measured in step (b) is lower than the level of the one or more metabolites in the subject suffering from mild cognitive impairment,
wherein the one or more metabolites is selected from the group consisting of 12.94.5, 8.93.65, 8.89.433, 9.29.925, and 8.14.983.

17. A method for monitoring an efficacy of a treatment of cognitive impairment in a subject, the method comprising:
(a) obtaining a first sample from the subject before the treatment and a second sample from the subject during or after treatment;
(b) measuring a first level of a metabolite in the first sample and a second level of the metabolite in the second sample, wherein
(i) the metabolite is selected from the group consisting of TRP, 5-HTP:TRP, XAN:HX and GSH:MET; or
(ii) the metabolite is selected from the group consisting of 5-HIAA, I-3-AA, KYN, 5-HIAA:5-HTP, KYN:TRP, I-3-AA:TRP, VMA, XANTH, HX, URIC, URIC:XAN, XAN:XANTH, MET, 15-65.533, and 8-93.65;
(c) comparing the first level of the metabolite and the second level of the metabolite wherein
(i) a second level of the metabolite of (b)(i) during or after treatment is higher than the first level of the metabolite of (b)(i) before treatment and is indicative of a therapeutic effect of the treatment in the subject; or
(ii) a second level of the metabolite of (b)(ii) during or after treatment is lower than the first level of the metabolite of (b)(ii) before treatment and is indicative of a therapeutic effect of the treatment in the subject.

18. The method of claim 17, wherein the cognitive impairment is selected from the group consisting of Alzheimer's Disease and mild cognitive impairment.

19. A kit for diagnosing cognitive impairment in a subject, the kit comprising reagents for detecting one or more metabolites selected from the group consisting of 5-HIAA, 5-HTP, I-3-AA, KYN, TRP, VMA, XANTH, XAN, URIC, HX, MET, GSH, 15-65.533, 8-93.65, 12.94.5, 8.93.65, 8.89.433, 9.29.925, and 8.14.983.

20. The kit of claim 19, wherein the cognitive impairment is selected from the group consisting of Alzheimer's Disease and mild cognitive impairment.
